(19) United States
(12) Patent Application Publication
(10) Pub. No.: US 2024/0173124 A1
(43) Pub. Date: May 30, 2024

(54) PROSTHETIC VALVES WITH MECHANISMS FOR CONTROLLED EXPANSION

(71) Applicant: EDWARDS LIFESCIENCES CORPORATION, Irvine, CA (US)

(72) Inventors: Noam Nir, Pardes-Hanna (IL); Michael Bukin, Pardes Hanna (IL); Ofir Witzman, Harish (IL); Oren Cohen, Kadima (IL)

(21) Appl. No.: 18/422,384

(22) Filed: Jan. 25, 2024

Related U.S. Application Data
(63) Continuation of application No. PCT/US2022/038527, filed on Jul. 27, 2022.
(60) Provisional application No. 63/338,837, filed on May 5, 2022.
(60) Provisional application No. 63/228,907, filed on Aug. 3, 2021.

(51) Int. Cl. A61F 2/24

(52) U.S. Cl. CPC A61F 2/2418; A61F 2/2439; A61F 2250/0021

(57) ABSTRACT

This disclosure is directed to self-expanding prosthetic heart valves having resistance-based mechanisms to control the rate of self-expansion of the prosthetic heart valves. In some examples, the prosthetic heart valve can be configured to radially self-expand to a partially radially expanded state, and can then be further radially expanded to the radially expanded state and/or locked in the radially expanded state by pulling an actuation member of the expansion and locking assembly. In other examples, the prosthetic heart valve can be configured to self-expand from the radially compressed state to the fully radially expanded state without mechanical actuation. In some examples, the resistance-based expansion control mechanisms can be disposed between delivery system actuators and valve frame components. In other examples, the resistance-based expansion control mechanisms can be disposed between delivery system actuators and other components of the delivery system.

### CROSS-REFERENCE TO RELATED APPLICATIONS

This application is a continuation of International Application No. PCT/US2022/038527, filed Jul. 27, 2022, which claims benefit of U.S. Provisional Application No. 63/228,907, filed on Aug. 3, 2021, and U.S. Provisional Application No. 63/338,837, filed on May 5, 2022, the contents of each of which are herein incorporated by reference in their entirety.

### FIELD

The present disclosure relates to expandable prosthetic heart valves, and to methods, assemblies, and apparatuses for delivering and deploying (e.g., positioning, radially expanding, locking, etc.) and controlling the deployment of such prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic heart valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

A drawback of self-expandable prosthetic heart valves is that they expand very rapidly when released from a delivery sheath within the vasculature of a patient, which can cause trauma to the implantation site, or cause the prosthetic heart valve to become misaligned during implantation.

Accordingly, there is a need for improved self-expanding prosthetic heart valve, as well as delivery apparatus and methods for implanting improved self-expandable prosthetic heart valves.

### SUMMARY

Disclosed herein are prosthetic heart valves and prosthetic heart valve delivery assemblies having friction members. The friction members can be used to control the rate of expansion of the prosthetic heart valves in which they are included. A controlled rate of expansion may be useful to reduce or eliminate undesirable jumping of the prosthetic heart valve within the vasculature of a patient during the implantation process. In some examples, the friction member can engage with a component of the prosthetic heart valve frame. In other examples, the friction member can engage with a component of the prosthetic heart valve delivery apparatus. Also disclosed herein are methods for using the prosthetic heart valves having friction members disclosed.

Certain examples concern a medical assembly comprising a prosthetic heart valve having a radially expandable annular frame. The frame comprises at least one frame portion defining an axially extending channel, a valvular structure disposed within the frame and configured to regulate the flow of blood through the frame in one direction, and at least one actuation member coupled to the frame and configured to apply an axially directed force to the frame. The prosthetic heart valve is configured to self-expand from a radially compressed state to at least a partially radially expanded state. The medical assembly also comprises a delivery device. The delivery device comprises at least one delivery system actuator releasably coupled to the actuation member and extending through the axially extending channel of the frame. One of an exterior surface of the delivery system actuator and an inner surface the axially extending channel comprises a friction surface and the other of the exterior surface of the delivery system actuator and the inner surface of the axially extending channel comprises an opposing surface that can engage the friction surface, wherein the exterior surface of the delivery system actuator and the inner surface of the axially extending channel have a first coefficient of friction when the friction surface and the opposing surface engage each other and a second coefficient of friction when the friction surface and the opposing surface do not engage each other, wherein the first coefficient of friction is greater than the second coefficient of friction. When the prosthetic heart valve self-expands from the radially compressed state to the partially radially expanded state, the delivery system actuator can slide relative to the axially extending channel and the friction surface slides against the opposing surface to control a rate of expansion of the prosthetic heart valve. The delivery system actuator is configured to move the actuation member in an axial direction to further expand the prosthetic heart valve from the partially radially expanded state to a further radially expanded state.

Certain examples concern a medical assembly, comprising a prosthetic heart valve. The prosthetic heart valve comprises a radially expandable annular frame, a valvular structure disposed within the frame and configured to regulate the flow of blood through the frame in one direction, and at least one actuation member coupled to the frame and configured to apply an axially force to the frame. The frame is configured to self-expand from a radially compressed state to at least a partially radially expanded state. The medical assembly also comprises a delivery device. The delivery device comprises at least one delivery system actuator releasably coupled to the actuation member and at least one support tube disposed around the delivery system actuator and configured to allow the delivery system actuator to extend and move axially therethrough. One of an exterior surface of the delivery system actuator and an inner surface of the support tube comprises a resistance surface and the other of the exterior surface of the delivery system actuator and the inner surface of the support tube comprises an opposing surface that can engage the resistance surface, wherein a coefficient of friction between the delivery system actuator and the support tube is increased when the resistance surface and the opposing surface engage each other. When the frame self-expands from the radially compressed state to the partially radially expanded state, the delivery system actuator slides relative to the support tube and the resistance surface slides against the opposing surface to control a rate of expansion of the frame. The delivery system actuator is configured to move the actuation member in an axial direction to further expand the frame from the partially radially expanded state to a further radially expanded state.

Certain examples concern a prosthetic heart valve, comprising a radially expandable frame. The frame comprises a frame portion defining at least one axially extending channel, an axially extending frame member that extends through the axially extending channel, and a plurality of leaflets disposed within the frame and configured to regulate the flow of blood through the frame in one direction. One of an exterior surface of the axially extending frame member and an inner surface the axially extending channel comprises a friction surface and the other of the exterior surface of the axially extending frame member and the inner surface of the axially extending channel comprises an opposing surface that can engage the friction surface, wherein a friction force between the axially extending frame member and the axially extending channel is increased when the friction surface is engaged with the opposing surface. The prosthetic heart valve is configured to self-expand from a radially compressed state to at least a partially radially expanded state.

Certain examples concern a medical assembly comprising a prosthetic heart valve. The prosthetic heart valve comprises a radially expandable annular frame. The frame comprises a plurality of frame portions defining a plurality of axially extending channels. The prosthetic heart valve also comprises a valvular structure disposed within the frame and configured to regulate the flow of blood through the frame in one direction and a plurality of actuation members each having a distal end coupled to the frame and configured to apply an axial force to the frame. The prosthetic heart valve is configured to self-expand from a radially compressed state to at least a partially radially expanded state. The medical assembly also comprises a delivery device. The delivery device comprises a plurality of delivery system actuators releasably coupled to the plurality of actuation members and extending through the plurality of channels of the frame and a plurality of support tubes disposed around the plurality of delivery system actuators and configured to allow the plurality of delivery system actuators to extend and move axially therethrough. One of a plurality of exterior surfaces of the plurality of delivery system actuators, a plurality of inner surfaces of the plurality of axially extending channels, or a plurality of inner surfaces of the plurality of support tubes comprise a plurality of resistance surfaces and the medical assembly further comprises a corresponding plurality of opposing surfaces that can engage the resistance surfaces, wherein a force resisting an axial movement of the delivery system actuators is increased when the resistance surface is engaged with the opposing surface. When the prosthetic heart valve self-expands from the radially compressed state to the partially radially expanded state, the plurality of delivery system actuators can slide relative to the plurality of axially extending channels or the plurality of support tubes and the plurality of resistances surfaces slide against the opposing surfaces to control a rate of expansion of the prosthetic heart valve.

Certain examples concern a delivery device, comprising at least one delivery system actuator, at least one support tube disposed around the delivery system actuator and configured to allow the delivery system actuator to extend and move axially therethrough. One of an exterior surface of the delivery system actuator or an interior surface of the support tube comprise a friction surface and the other of the exterior surface of the delivery system actuator comprises an opposing surface that engages the friction surface.

Certain examples concern a method of implanting a radially expandable prosthetic heart valve, comprising inserting a distal end portion of a delivery device and a prosthetic heart valve into the vasculature of a patient, while the prosthetic heart valve is in a radially compressed state, positioning the prosthetic heart valve within or adjacent a desired implantation site and allowing the prosthetic heart valve to radially self-expand from the radially compressed state to at least a partially radially expanded state. When the prosthetic heart valve is self-expanding, a friction surface of the delivery device slidably engages an opposing surface to create sliding friction that opposes the self-expansion of the radially expandable prosthetic heart valve.

Certain examples concern a medical assembly comprising a prosthetic heart valve. The prosthetic heart valve comprises a radially expandable annular frame, wherein the frame comprises at least one frame portion defining an axially extending channel, a valvular structure disposed within the frame and configured to regulate the flow of blood through the frame in one direction, at least one actuation member having a distal end coupled to the frame and configured to apply an axial force to the frame. The prosthetic heart valve is configured to self-expand from a radially compressed state to at least a partially radially expanded state. The medical assembly also comprises a delivery device. The delivery device comprises at least one delivery system actuator releasably coupled to the actuation member and extending through the axially extending channel of the frame. One of an exterior surface of the delivery system actuator and an inner surface the axially extending channel comprises a friction surface and the other of the exterior surface of the delivery system actuator and the inner surface of the axially extending channel comprises an opposing surface that can engage the friction surface. A coefficient of friction between the delivery system actuator and the axially extending channel is increased when the friction surface is engaged with the opposing surface. The frame has an elastic radial expansion force when in the radially compressed state that causes the prosthetic heart valve to self-expand from the radially compressed state to the partially radially expanded state, and the friction surface partially opposes the clastic radial expansion force to permit self-expansion of the frame at a rate lower than a free expansion rate. The delivery system actuator is configured to move the actuation member in an axial direction to further expand the prosthetic heart valve from the partially radially expanded state to a further radially expanded state.

Certain examples concern a prosthetic valve, comprising a radially expandable annular frame, a valvular structure disposed within the frame and configured to regulate flow of blood through the frame in one direction, at least one actuation member rotatably coupled to the frame and comprising an actuation member aperture, and a flexible tension member extending through the actuation member aperture and around the frame.

Certain examples concern a medical assembly comprising a prosthetic heart valve. The prosthetic heart valve comprises a radially expandable annular frame, a valvular structure disposed within the frame and configured to regulate flow of blood through the frame in one direction, at least one actuation member rotatably coupled to the frame and comprising an actuation member aperture, and a flexible tension member extending through the actuation member aperture and around the frame. The medical assembly further comprises a delivery device. The delivery device comprises at least one delivery system actuator.

Certain examples concern a prosthetic valve, comprising a radially expandable annular frame, a valvular structure disposed within the frame and configured to regulate flow of blood through the frame in one direction, at least one actuation member rotatably coupled to the frame and comprising a terminal end and an actuation member slot extending from the terminal end, and a flexible tension member extending through the actuation member slot and around the frame.

Certain examples concern a medical assembly comprising a prosthetic heart valve. The prosthetic heart valve comprises a radially expandable annular frame, a valvular structure disposed within the frame and configured to regulate flow of blood through the frame in one direction, and a flexible tension member extending around the frame. The medical assembly further comprises a delivery device. The delivery device comprises at least one delivery system actuator comprising a terminal end and an actuation member slot extending from the terminal end.

Certain examples concern a medical assembly comprising a prosthetic heart valve. The prosthetic heart valve comprises a radially expandable annular frame, a valvular structure disposed within the frame and configured to regulate flow of blood through the frame in one direction, and a flexible tension member. The frame comprises at least one non-commissural vertical post that comprises a channel extending therethrough between an aperture and a proximal end thereof. The flexible tension members extends around the frame and forms a local loop that extends through the channel. The medical assembly further comprises a delivery device. The delivery device comprises a delivery system shaft defining a lumen, and a resistance feature disposed within the lumen and defining an internal passage that comprises a friction surface.

Certain examples concern a medical assembly comprising a prosthetic heart valve. The prosthetic heart valve comprises a radially expandable annular frame, a valvular structure disposed within the frame and configured to regulate flow of blood through the frame in one direction, and a flexible tension member extending around the frame and comprising a first end portion coupled to the frame, and a second end portion opposite to the first end portion. The medical assembly further comprises a delivery device. The delivery device comprises a delivery system shaft defining a lumen, and a resistance feature disposed within the lumen and defining an internal passage that comprises a friction surface.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

The foregoing and other objects, features, and advantages of the invention will become more apparent from the following detailed description, which proceeds with reference to the accompanying figures.

The foregoing and other objects, features, and advantages of the invention will become more apparent from the following detailed description, which proceeds with reference to the accompanying figures.

### BRIEF DESCRIPTION OF THE DRAWINGS

FIG. 1 depicts a perspective view of a self-expanding prosthetic heart valve according to one example.

FIG. 2A depicts the frame of the self-expanding prosthetic heart valve of FIG. 1 in a fully radially expanded configuration.

FIG. 2B depicts the frame of the self-expanding prosthetic heart valve of FIG. 1 in a partially radially expanded configuration.

FIG. 2C depicts the frame of the self-expanding prosthetic heart valve of FIG. 1 in a radially compressed configuration.

FIG. 3 depicts a prosthetic heart valve delivery device according to one example.

FIG. 4A depicts a prosthetic heart valve delivery device in use with a prosthetic heart valve according to one example, showing the valve in the fully radially expanded configuration.

FIG. 4B depicts a prosthetic heart valve delivery device in use with a prosthetic heart valve according to one example, showing the valve in the partially radially expanded configuration.

FIG. 4C depicts a prosthetic heart valve delivery device in use with a prosthetic heart valve according to one example, showing the valve in the radially compressed configuration inside a retaining sheath.

FIG. 5 depicts a prosthetic heart valve delivery device and a prosthetic heart valve retained in the radially compressed configuration by an adjustable loop.

FIG. 6 shows a portion of a frame of a prosthetic heart valve having an actuation member and a channel through a proximal apex portion of the frame.

FIG. 7A shows the proximal apex portion of the prosthetic heart valve frame of FIG. 6, showing the channel and a locking mechanism in greater detail.

FIG. 7B shows the proximal apex portion of the prosthetic heart valve frame of FIG. 6, showing the channel and a locking mechanism deflected into the locked position in greater detail.

FIG. 8 shows a portion of a frame of a prosthetic heart valve having an actuation member connected to an actuator of a delivery device.

FIG. 9 shows the proximal apex portion of the prosthetic heart valve frame of FIG. 8, showing the connected actuation member and actuator of a delivery device drawn into an apex channel.

FIG. 10 shows a portion of a frame of a prosthetic heart valve having a locking mechanism according to another example.

FIG. 11 shows a delivery system actuator with a valve expansion resistance feature according to one example.

FIG. 12 shows a cross-section of the delivery system actuator of FIG. 11

FIG. 13 show a delivery system actuator with a valve expansion resistance feature according to another example.

FIG. 14 shows a cross-section of the delivery system actuator of FIG. 13.

FIG. 15 shows a top-down cross-sectional view of a stationary member of a frame or delivery system component having a channel and a delivery system actuator with a valve expansion resistance feature passing through the channel, according to one example.

FIG. 16 shows a cross-section of the stationary member and delivery system actuator taken along line 16-16 of FIG. 15.

FIG. 17 shows a cross-sectional view of the stationary member and delivery system actuator of FIG. 15 when the prosthetic heart valve has been partially self-expanded.

FIG. 18 shows a cross-sectional view of the stationary member and delivery system actuator of FIG. 15 when the prosthetic heart valve has been fully self-expanded.

FIG. 19 shows a top-down cross-sectional view of a stationary member of a frame or a delivery system component having a channel and a delivery system actuator with a valve expansion resistance feature passing through the channel, according to one example.

FIG. 20 shows a cross-sectional view of the stationary member and delivery system actuator taken along line 20-20 of FIG. 19.

FIG. 21 shows a top-down cross-sectional view of a stationary member of a frame or a delivery system component having a channel and a delivery system actuator with a valve expansion resistance feature passing through the channel, according to one example.

FIG. 22 shows a cross-sectional view of the stationary member and delivery system actuator taken along line 22-22 of FIG. 21.

FIG. 23 shows a cross-sectional top-down view of a stationary member of a frame or a delivery system component having a channel and a delivery system actuator with a valve expansion controlling resistance feature passing through the channel, according to one example.

FIG. 24 shows a cross-sectional view of the stationary member and delivery system actuator taken along line 24-24 of FIG. 23.

FIG. 25A depicts the frame of another exemplary self-expanding prosthetic heart valve in a fully radially expanded configuration.

FIG. 25B depicts the frame of FIG. 25A in a partially radially expanded configuration.

FIG. 26A depicts the frame of another exemplary self-expanding prosthetic heart valve in a partially radially expanded configuration.

FIG. 26B depicts the frame of FIG. 26A in a fully radially expanded configuration.

FIG. 27 depicts the frame of another exemplary self-expanding prosthetic heart valve in a fully radially expanded configuration, with a delivery device engaged therewith.

FIG. 28A shows a cross-sectional view of a portion of the frame and delivery device of FIG. 27 when the prosthetic heart valve is compressed or partially self-expanded.

FIG. 28B shows a cross-sectional view of a portion of the frame and delivery device of FIG. 27 when the prosthetic heart valve is further expanded or fully self-expanded.

FIG. 29 depicts a perspective view of the self-expanding prosthetic heart valve of FIG. 27, with an outer skirt.

FIG. 30A depicts the frame of another exemplary self-expanding prosthetic heart valve in a partially radially expanded configuration, with a delivery device engaged therewith.

FIG. 30B depicts the frame of FIG. 30A in a fully radially expanded configuration.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

### INTRODUCTION

Disclosed herein are various examples of self-expanding prosthetic heart valves for replacement of the native heart valve of a patient. The prosthetic heart valves disclosed herein can be radially compressed and/or expanded, as well as locked in place, by an expansion and locking mechanism. As one example, the prosthetic heart valves can be crimped on or retained by a delivery apparatus or restraining mechanism in a radially-compressed state during delivery and then radially expanded (and axially shortened) to a radially-expanded state once the prosthetic heart valve reaches the implantation site (or a location adjacent an implantation site). Various examples of the prosthetic heart valves disclosed herein can be self-expanding prosthetic heart valves, which expand from the radially-compressed state to a partially-expanded or fully-expanded state when released from the delivery apparatus or restraining mechanism.

Self-expanding prosthetic heart valves can present several challenges. Specifically, the self-expansion of these prosthetic heart valves, when released from the delivery system and/or the restraining mechanism, can occur too rapidly. Rapid expansion can cause self-expanding prosthetic heart valves to “jump” (rapid movement of the prosthetic valve relative to the delivery apparatus) or become misaligned with the native heart valve of the patient.

To overcome these challenges, it is possible to slow the expansion of self-expanding prosthetic heart valves by introducing a source of friction that resists radial expansion of the prosthetic heart valve by impeding the motion of the relevant components and allowing the self-expanding prosthetic heart valve to expand in a controlled and steady manner. This resistance can be achieved by introducing a friction-generating surface between two components of the prosthetic heart valve that are configured to move relative to one another during self-expansion of the prosthetic heart valve. For example, certain disclosed prosthetic heart valves may include a channel extending from an apex of the prosthetic heart valve through which an actuator or pull member can extend, and a friction surface can be disposed between the channel and the actuator or pull member. The desired resistance can also be achieved by a friction generating source disposed between certain components of the delivery apparatus that move relative to one another, such as the actuators and actuator sleeves of the delivery apparatus.

In the case of partially self-expanding prosthetic heart valves which self-expand to a partially expanded configuration and must thereafter be mechanically expanded to the desired fully-expanded configuration, it may be particularly advantageous for the friction sources to apply friction only during a self-expansion range of the prosthetic heart valve, and to apply no friction during a mechanical expansion step of the implantation procedure, as friction applied during mechanical expansion of the prosthetic heart valve can potentially interfere with the expansion of the prosthetic heart valve in the working range of the prosthetic heart valve, during which such resistance may not be useful.

This disclosure describes friction members or friction elements that are designed to slow the self-expansion of self-expanding prosthetic heart valves and heart valve frames. These fiction members can be disposed between two components of the prosthetic heart valve frame movable relative to one another, or between a component of the prosthetic heart valve frame and a component of the delivery apparatus, such as an actuator, or between two components of the delivery apparatus. The friction members can be included either on heart valve frame components, or components of a prosthetic heart valve delivery apparatus, such as a delivery apparatus actuator.

Also disclosed herein are prosthetic heart valve frames incorporating such friction members. Related delivery apparatus and methods of implanting and using such prosthetic heart valves are also described herein. This can, for example, provide a controlled and stable self-expansion of self-expanding prosthetic heart valves and thereby avoid undesirable “jumping” caused by rapid self-expansion. This can, among other things, help to ensure that the prosthetic heart valve is implanted in a precise and controlled manner while avoiding trauma to the patient. In this way, the friction members, prosthetic heart valves, delivery apparatus, and methods of use disclosed herein can overcome many of the challenges facing self-expanding prosthetic heart valves.

The prosthetic heart valves, delivery apparatus, and methods disclosed herein may be described in relation to a particular implantation location (e.g., a native aortic valve) and/or using a particular delivery procedure (e.g., transfemoral delivery). These implantation locations and delivery procedures are merely examples. The disclosed devices and methods can be adapted to various other implantation locations (e.g., a native mitral valve, tricuspid valve, and/or pulmonary valve) and/or other delivery procedures (e.g., transapical, transseptal, etc.).

### The Disclosed Technology

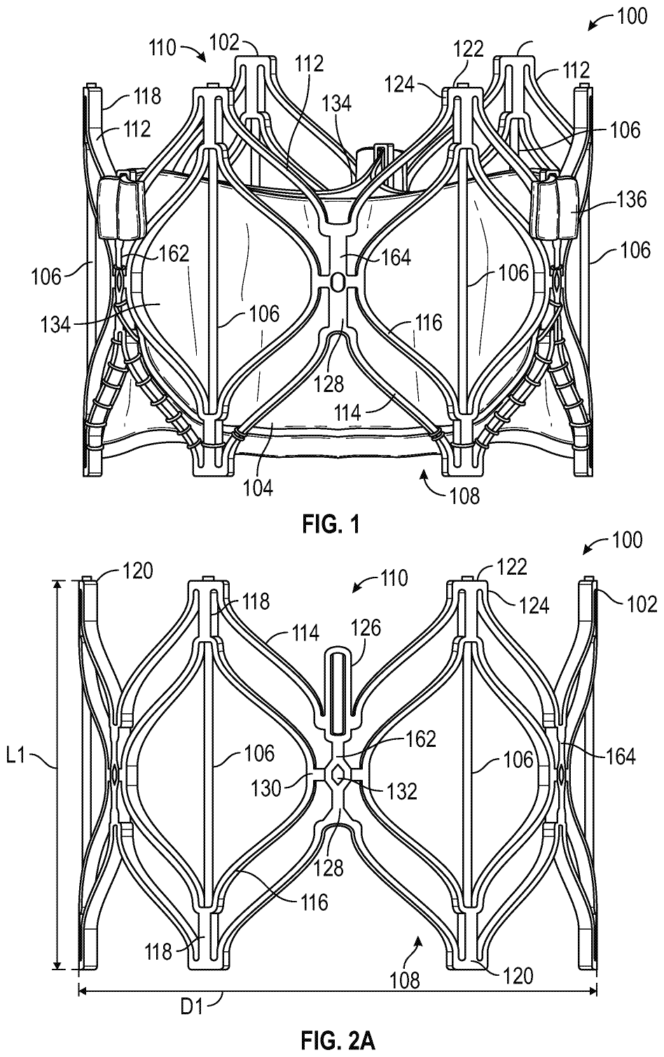

FIG. 1 depicts one example of a prosthetic heart valve, which can include an expansion resistance feature to control expansion of the prosthetic valve, as further described below. The prosthetic heart valve 100 (also referred to herein as “the prosthetic valve 100”) comprises a frame 102 and a valvular structure 104.

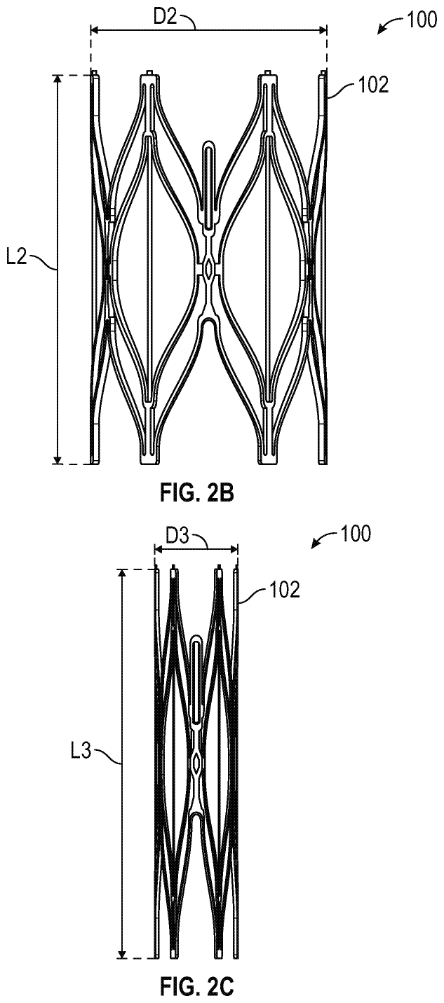

The frame 102 (which can also be referred to as “a stent” or “a support structure”) can be configured for supporting the valvular structure 104 and for securing the prosthetic heart valve 100 within a native heart valve and/or within another support structure (e.g., an anchoring frame (such as a coil) and/or a previously implanted prosthetic heart valve (i.e., in a valve-in-valve procedure). The frame 102 may be partially self-expanding or fully self-expanding. In the case of a partially self-expanding frame, the frame may be configured to expand from a compressed or delivery configuration, illustrated in FIG. 2C, to an intermediate or partially-expanded configuration, such as the configuration illustrated in FIG. 2B, under the inherent resiliency or elastic force of the fame. In the case of partially self-expanding frames, the frame 102 can further include actuation members 106 (e.g., six actuators in the illustrated example) which are coupled to the frame 102 and are configured to adjust expansion of the frame 102 from a partially-expanded configuration (e.g., FIG. 2B) to a plurality of configurations including one or more functional or expanded configurations (e.g., FIG. 2A).

In examples of the frame which are fully self-expanding, the resiliency of the frame is sufficient to expand the frame to the fully radially expanded state, such as that shown in FIG. 2A. In the case of fully self-expandable frames, prosthetic valve need not include the actuator members 106, or alternatively, the actuators can be included for mounting a friction member but do not need to be used for applying expansion forces to the frame, as further described below. It should be noted that the valvular structure 104 of the prosthetic heart valve 100 is not shown FIGS. 2A-2C to better illustrate other components of the prosthetic heart valve 100.

Referring now to FIGS. 1-2A, the frame 102 of the prosthetic heart valve 100 has a first end 108 and a second end 110. In the depicted orientation, the first end 108 of the frame 102 is an inflow end and the second end 110 of the frame 102 is an outflow end. In other examples, the first end 108 of the frame 102 can be the inflow end and the second end 110 of the frame 102 can be the outflow end.

The frame 102 includes a plurality of struts 112, which are interconnected. In some examples, the struts can define a plurality of cells. For example, in the illustrated example, the struts 112 define a row of six primary cells 114. The frame 102 also comprises a row of six secondary cells 116, which are each nested with a respective primary cell 114. Accordingly, the primary cells and the secondary cells can also be referred to as “outer cells” and “inner cells,” respectively. The primary cells 114 and the secondary cells 116 are interconnected at their ends by vertical struts 118 (which can also be referred to as “non-pivoting struts”). The primary cells 114 and the secondary cells 116 each comprise a tear-drop like shape, which also resembles a hexagonal shape but with curved side. Portions of the primary cells and the secondary cells can also be described as having a “wishbone” shape. As such, the primary and secondary cells of the frame may be described as forming a “parallel wishbone” or “double wishbone” configuration. The primary and/or secondary cells can comprise various other shapes in other examples.

The primary cells 114 and/or the vertical struts 118 of the frame 102 form apices 120 at the first end 108 and the second end 110 of the frame 102. In the depicted example, each apex 120 comprises a “T” shape defined by a respective vertical strut 118 and a pair of angled struts of a primary cell 114. Each apex 120 comprises a flat (or at least substantially flat) end surface 122 extending between two vertically-oriented side surfaces 124. In other examples, the apices of the frame can comprise various other shapes (e.g., rounded).

The frame can further comprise a plurality of leaflet attachment structures. For example, as depicted in FIG. 2A, the frame 102 comprises a plurality of commissure windows 126 disposed circumferentially between adjacent pairs of the primary cells 114 of the frame. The commissure windows 126 are spaced axially from the apices 120 at the second end 110 of the frame 102 toward the first end 108 of the frame. In other examples, the commissure windows can be disposed at various other axial positions relative to the apices of the frame (e.g., axially aligned with the apices at the second end of the frame). In the depicted example, the commissure windows 126 comprise an opening that is bounded on all sides in a “closed” configuration. In other examples, the commissure windows can comprise an “open” configuration (e.g., a U-shaped slot). The commissure windows 126 extend from vertical struts 128 in a cantilevered manner. In other examples, the commissure windows 126 can be supported at multiple locations in a non-cantilevered manner. The number of vertical struts 128 can be greater than the number of commissure windows 126, such that the vertical struts 128 can include commissural vertical struts 162, defined as vertical struts 128 that include commissure windows 126 extending therefrom, and non-commissural vertical struts 164, defined as vertical struts 128 that do not include commissure windows extending therefrom.

The frame 102 can comprise various other struts and/or openings. For example, the frame 102 comprises a pair of circumferentially-extending struts 130 (which may also be referred to as “laterally-extending struts”) extending from the vertical struts 128 of the secondary cells 116. The frame 102 also comprises apertures 132 disposed in the vertical struts 128.

The struts 112 of the frame 102 are configured such that the frame 102 can move between a plurality of radial configurations. For example, FIG. 2A depicts a fully radially-expanded configuration, FIG. 2B depicts a partially radially-expanded configuration, and FIG. 2C depicts a radially-compressed configuration. The depicted configurations are exemplary, and the frame can be expanded or compressed to a lesser or greater extent than depicted. As the frame moves between the various configurations, some of the struts of the frame deflect or pivot relative to each other. For example, the angled struts (which can also be referred to as “diagonal struts”) (i.e., the non-vertically and non-horizontally oriented struts) deflect relative to the vertically and horizontally oriented struts. In this manner, the frame of the prosthetic heart valve axially elongates when the frame is radially compressed and axially foreshortens when the frame is radially expanded. Thus, a diameter D1 of the prosthetic heart valve 100 in the radially-expanded state (FIG. 2A) is greater than a diameter D2 of the prosthetic heart valve 100 in the partially radially-expanded state (FIG. 2B) and a diameter D3 of the prosthetic heart valve 100 in the radially-compressed state (FIG. 2C), and the diameter D2 of the prosthetic heart valve 100 in the partially radially-expanded state is greater than the diameter D3 of the prosthetic heart valve 100 in the radially-compressed state (i.e., D1>D2>D3). Oppositely, a length L1 of the prosthetic heart valve 100 in the radially-expanded state is less than a length L2 of the prosthetic heart valve 100 in the partially radially-expanded state and a length L3 of the prosthetic heart valve 100 in the radially-compressed state, and the length L2 of the prosthetic heart valve 100 in the partially radially-expanded state is less than the length L3 of the prosthetic heart valve 100 in the radially-compressed state (i.e., L1<L2<L3).

In one specific example, a prosthetic heart valve according to the general examples illustrated in FIGS. 2A-2C can have a diameter of 7 mm in the compressed or delivery state, shown in FIG. 2C. In such an example, the prosthetic heart valve can be configured to self-expand to an intermediate diameter, illustrated in FIG. 2B, of 20 mm, and to be further mechanically expanded to a fully radially expanded diameter of 29 mm. It is to be understood however, that the diameter of the prosthetic heart valve in the compressed state, the partially radially expanded state, and the fully radially expanded state can vary according to the needs of an individual patient or implantation procedure.

The frame can be formed of a shape memory material (e.g., a nickel titanium alloy, such as nitinol) such that the frame can be shape-set to a particular configuration and then elastically deformed to one or more other configurations. As one example, the frame 102 is formed of nitinol and shape-set in the partially radially-expanded configuration (e.g., FIG. 2B). The frame 102 can be elastically deformed to the radially-compressed configuration (e.g., FIG. 2C) and to the radially-expanded configuration (e.g., FIG. 2A). The frame can be elastically deformed to the depicted configuration and/or various other configurations, for example, by using the actuation members 106, a delivery apparatus, and/or a crimping device), as further described below.

In other examples, the frame can be formed of a plastically-deformable material (e.g., stainless steel or cobalt chromium alloy) such that the frame can be formed in a particular configuration and then plastically deformed to one or more configurations which are radially smaller or larger than the configuration in which the frame is formed.

In some examples, the frame 102 can be formed from a single piece of material (e.g., a metal tube). This can be accomplished, for example, via laser cutting, electroforming, and/or physical vapor deposition. In other examples, the frame can be constructed by forming individual components coupling the individual components together (e.g., via welding, brazing, and/or other means for bonding).

Referring again to FIG. 1, the valvular structure 104 of the prosthetic heart valve 100 is coupled to the frame 102 (e.g., directly and/or indirectly via other components such a sealing skirt). The valvular structure 104 is configured to allow blood flow through the prosthetic heart valve 100 from the first end 108 to the second end 110 in an antegrade direction and to restrict blood from through the prosthetic heart valve 100 from the second end 110 to the first end 108 in a retrograde direction. The valvular structure can include various components including a leaflet assembly comprising one or more leaflets. For example, the valvular structure 104 in the illustrated example comprises a leaflet assembly having three leaflets 134.

The leaflets 134 of the prosthetic heart valve 100 can be made of a flexible material. For example, the leaflets 134 can be made from in whole or part, biological material, bio-compatible synthetic materials, or other such materials. Suitable biological material can include, for example, bovine pericardium, equine pericardium, porcine pericardium, and/or pericardium from other sources.

The leaflets 134 can be arranged to form commissures 136 (e.g., pairs of adjacent leaflets), which can, for example, be mounted to the frame at the commissure windows 126 (e.g., via sutures, fabric, adhesive, and/or other means for mounting). Further details regarding prosthetic heart valves, including the manner in which the valvular structure 104 can be coupled to the frame 102 of the prosthetic heart valve 100, can be found in U.S. Pat. Nos. 6,730,118, 7,393,360, 7,510,575, 7,993,394, 8,652,202, and 9,393,110, and U.S. Publication No. 2018/0325665, which are incorporated by reference herein.

Referring again to FIG. 1, partially self-expanding prosthetic heart valves may include actuation members. The actuation members 106 of the prosthetic heart valve 100 are mounted to and spaced circumferentially around the frame 102. In the illustrated example, the prosthetic heart valve 100 comprises six actuation members 106. In other examples, the prosthetic heart valve can comprise fewer or more than six actuation members (e.g., 1-5 or 7-15). The actuation members 106 are configured to, among other things, radially expand the frame 102 by pulling the distal end of the frame towards the proximal end of the frame. For this reason, the actuation members 106 can also be referred to as “pull members.” In some examples, the actuation members 106 can also be configured to radially compress the frame 102 by pushing the distal end of the frame away from the proximal end of the frame.

The actuation members can be formed of various materials and in various physical configurations. For example, in some instances, the actuation members can be a rod or shaft. In such instances, the actuation members can be formed as separate components from the frame, which are then coupled thereto (e.g., via welding, adhesive, fasteners, or other means for coupling). Alternatively, the actuation members and the frame can be integrally formed as a unitary structure (e.g., by forming the frame and actuation members from a tube). In other instances, the actuation members can be a cable, wire, cord, suture, or other relatively flexible material (i.e., compared to a shaft or rod). In such instances, the flexible actuation member can be coupled to the frame by tying or looping the actuators around the struts of the frame and/or by coupling the actuation member to the frame via a fastener (e.g., a grommet), adhesive, and/or other means for coupling.

In some examples, the actuation members are configured for rotational actuation. For example, an actuation member can comprise external threads along one or more portions of the actuation member (e.g., similar to a bolt or screw). A first end portion of the actuation member can be coupled to a first portion (e.g., an inflow end portion) of the frame (e.g., via the head of the screw) such that the actuation member can rotate relative to the first portion of the frame but is axially fixed thereto. A second end portion of the actuation member can be extended through a lumen of the frame disposed at another location (e.g., an inflow end portion) of the frame. The lumen of the frame can comprise corresponding internal threads configured to mate with the external threads of the actuation member. In this manner, rotating the actuation member in a first direction (e.g., clockwise) relative to the frame results in radial expansion of the frame as the first end portion of the frame and the second end portion of the frame move axially toward each other along the threads of the actuation member. Likewise, rotating the actuation member in a second direction (e.g., counterclockwise) relative to the frame results in radial compression of the frame as the first end portion of the frame and the second end portion of the frame move axially away from each other along the threads of the actuation member. Due to the threaded engagement between the frame and the actuation members, the actuation members lock the frame at a desired expanded configuration when the actuation members are stationary relative to the frame. Accordingly, such actuation members can also be referred to as “lockers,” “locking members,” or “locking mechanisms.” Such rotational actuation members, however, have their shortcomings. For example, forming the actuation members and frame, which are very small components, with threads can present manufacturing and reliability challenges. To allow for partial self-expansion of the frame, the threads of the actuation member can be configured to engage the threads of the frame only when the frame reaches the end of the self-expansion range of the frame. Within the self-expansion range, the actuator member can freely slide through the lumen of the frame.

Accordingly, in other examples, the actuation members are configured for linear actuation. In such instances, the actuation members 106 comprise fixed end portions fixedly coupled to one portion of the frame (e.g., the first end portion) and free end portions movably coupled to another component of the frame or of a delivery assembly (e.g., a delivery system actuator of a delivery apparatus). For example, the fixed end portions of the actuators can be coupled to and/or extend axially from the vertical struts 118 at the inflow end portion of the frame 102, across the primary and second cells and through a lumen traversing the vertical struts 118 at the outflow end portion of the frame 102. The actuation members 106 can be used to expand the frame 102 by pulling the actuation members 106 toward the outflow end portion of the frame while applying an opposing force on the apices of the outflow end portion of the frame (e.g., with a delivery apparatus). These axially-opposing forces together apply a compressive force to the frame and result in radial expansion of the frame. The frame can be radially compressed by reducing tension on the actuators and allowing the elastic properties of the frame to radially compress the frame to its neutral or resting state and/or by an external radially inward force (e.g., a crimping device and/or native anatomy within a patient's body). Frames with linear actuation provide one or more advantages, including improved manufacturability and reliability relative to frames with rotational actuation. Despite these advantages, frames with linear actuation require a locking mechanism to retain the actuation member's position relative to the frame in order to secure the frame in one or more desired configurations. Some examples of actuators and locking mechanisms are described further below.

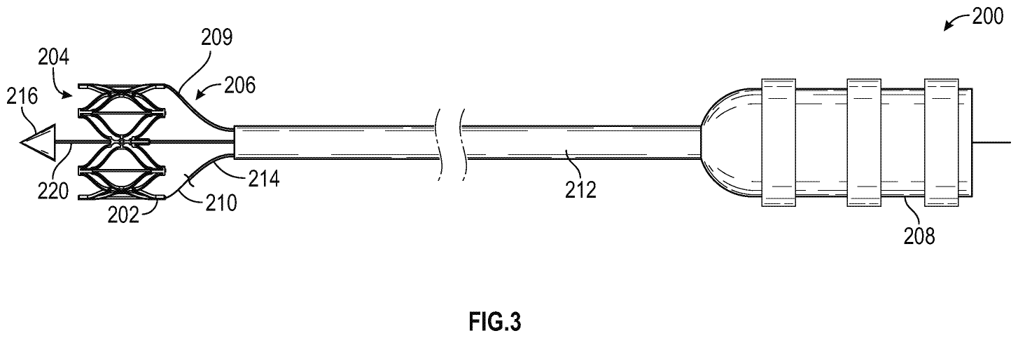

Each of the actuators can be configured to form a releasable connection with one or more respective delivery system actuators of a delivery apparatus. This releasable connection can, for example, include a threaded connection, a plurality of interlocking shafts, and other means of forming a releasable connection. One example of a releasable connection between the actuation members and the delivery system actuators of a delivery apparatus is illustrated in FIG. 3. During the implantation procedure, the prosthetic heart valve may be allowed to self-expand to the partially radially expanded state while the delivery system actuators are connected to the actuation members. Thereafter, the delivery actuators may pull axially on the actuation members to further bring the distal end of the prosthetic heart valve close to the proximal end of the prosthetic heart valve, causing a radial expansion of the prosthetic heart valve to the fully expanded configuration. Optionally, the delivery actuators may be configured to further act on the actuation members to cause adjustments in the radial size of the prosthetic heart valve, for example to adjust the installed prosthetic heart valve to accommodate the needs of a particular patient.

The prosthetic heart valves described herein can also comprise one or more optional components. For example, in some examples, a prosthetic heart valve can include one or more sealing skirts. For example, the prosthetic heart valve 100 can include an inner skirt mounted on the inner surface of the frame 102. The inner skirt can function as a sealing member to prevent or decrease paravalvular leakage, to anchor the leaflets to the frame, and/or to protect the leaflets 134 against damage caused by contact with the frame 102 during crimping and during operation of the prosthetic heart valve 100 (i.e., the opening and closing of the leaflets). The prosthetic heart valve 100 can also include an outer skirt mounted on the outer surface of the frame 102. The outer skirt can function as a sealing member for the prosthetic heart valve by sealing against the tissue of the native valve annulus and thus reducing paravalvular leakage around the prosthetic heart valve. The inner and outer skirts can be formed from any of various suitable biocompatible materials, including any of various synthetic materials (e.g., PET) and/or natural tissue (e.g., pericardial tissue). The inner and outer skirts can be mounted to the frame using sutures, adhesive, and/or other means for attaching the skirts to the frame.

The examples of prosthetic heart valves described herein may be configured to be implanted in the vasculature of a patient by a delivery apparatus. A suitable delivery apparatus may comprise an elongated shaft configured to pass through the vasculature of a patient, one or more delivery actuators to manipulate a prosthetic heart valve within the patient's body, and a control mechanism by which a physician may control the actuators. Some examples of a delivery apparatus may further include a restraining mechanism configured to retain the prosthetic heart valve in a compressed configuration.

FIG. 3 illustrates a delivery apparatus 200, according to one example, designed to advance a prosthetic heart valve 202 through a patient's vasculature and/or to deliver the prosthetic heart valve 202 to an implantation site (e.g., native heart valve) within a patient's body. The prosthetic heart valve 202 can be mounted on, retained within, and/or releasably coupled to a distal end portion of the delivery apparatus 200. The prosthetic valve 202 can represent the prosthetic valve 100 previously described herein and illustrated in FIG. 1.

The prosthetic heart valve 202 can include a distal end 204 (which can be the inflow end of the prosthetic heart valve 202, such as when the prosthetic heart valve 202 is configured to replace a defective aortic valve when delivered transfemorally) and a proximal end 206 (which can be the outflow end of the prosthetic heart valve 202, such as when the prosthetic heart valve 202 is configured to replace a defective aortic valve when delivered transfemorally), wherein the proximal end 206 is positioned closer to a handle 208 of the delivery apparatus 200 than the distal end 204, and wherein the distal end 204 is positioned farther from the handle 208 than the proximal end 206. It is to be understood that in other examples, such as when the prosthetic heart valve 202 is implanted in a different location in the vasculature of the patient, the proximal end 206 can alternatively be an inflow end of the prosthetic heart valve 202 and the distal end 204 can be an outflow end of the prosthetic heart valve 202.

The delivery apparatus 200 in the illustrated example generally includes the handle 208, a first shaft 212 (an outer shaft in the illustrated example) extending distally from the handle 208, a second shaft 220 (an inner shaft in the illustrated example) extending distally from the handle 208 through the first shaft 212, one or more delivery system actuators 210 extending distally through the outer shaft 212, and one or more support tubes (sometimes called support members) 214 that can extend distally through the outer shaft 212 and can abut the proximal end 206 of the prosthetic heart valve 202. The delivery apparatus 200 can further include a nose cone 216 connected to the distal end portion of the second shaft 220.

Each delivery system actuator 210 can have a distal end connected to the distal end of the prosthetic heart valve 202. Each of the delivery system actuators 210 can extend through a respective support tube 214 and together can define a respective actuator assembly 209 that can extend through the outer shaft 212 to the handle 208. In alternative examples, the delivery system actuators 210 and the support tubes 214 need not be co-axial with respect to each and instead can extend side-by-side through the shaft.

The delivery system actuators 210 and/or the support tubes 214 can be configured to radially expand the prosthetic heart valve 202 by bringing the ends 204, 206 of the prosthetic heart valve 202 closer together (i.e., squeezing the prosthetic heart valve 202 axially) thereby axially foreshortening and radially expanding the prosthetic heart valve 202. As one example, the delivery system actuators 210 can be configured to be actuated to provide a proximally directed (e.g., pulling) force to the distal end 204 of the prosthetic heart valve 202 while the one or more support tubes 214 can be configured to provide a countervailing distally directed (e.g., pushing) force to the proximal end 206 of the prosthetic heart valve 202. In another example, the delivery system actuators 210 can be configured to exert an axial force on a corresponding number of actuation members of the frame of the prosthetic heart valve (e.g., actuator members 106), which in turn may transmit the force to the distal end 204 of the prosthetic heart valve 202. In one such example, a physician can pull the delivery system actuators 210 to provide the proximally directed force to the distal end 204 of the prosthetic heart valve 202, while simultaneously gripping, holding, and/or pushing the handle 208 to provide the countervailing distally directed force to the proximal end 206 of the prosthetic heart valve 202.

As described in greater detail below, the delivery system actuators 210 can cooperate with a locking element on the prosthetic heart valve 202 to retain the prosthetic heart valve in a radially expanded state.

The delivery actuators can each comprise a suture, string, cord, wire, cable, rod or other similar device that can transmit a pulling force from the handle 208 to the prosthetic heart valve 202 valve when actuated by a physician. The support tubes 214 can comprise a relatively more rigid component, such a tube that can abut the proximal end 206 of the prosthetic heart valve 202 and resist proximal movement of the prosthetic heart valve relative to the shaft 212 when a proximal pulling force is applied to the delivery system actuators 210.

Although three pairs of delivery system actuators 210 and support tubes 214 are shown in FIG. 3, it should be understood that the delivery apparatus 200 can include more or less than three delivery system actuators 210 and/or three support tubes 214, in other examples. As just one example, the delivery apparatus 200 can include six delivery system actuators 210 and/or six support tubes 214. In other examples, a greater or fewer number of delivery system actuators 210 and/or support tubes 214 can be present, such as four, five, seven, and/or eight delivery system actuators 210 and/or four, five, seven, and/or eight support tubes 214. In some examples, the delivery apparatus 200 can include equal numbers of delivery system actuators 210 and support tubes 214. However, in other examples, the delivery apparatus 200 can include a different number of delivery system actuators 210 and support tubes 214.

Examples of the delivery apparatus disclosed herein may further include a restraining mechanism configured to retain the prosthetic heart valve in a compressed state. The restraining mechanism may be releasably attached to the prosthetic heart valve while the prosthetic heart valve is being advanced through the vasculature of the patient and/or being positioned at the desired implantation site, and may be detached one the prosthetic heart valve has been positioned in the desired location.

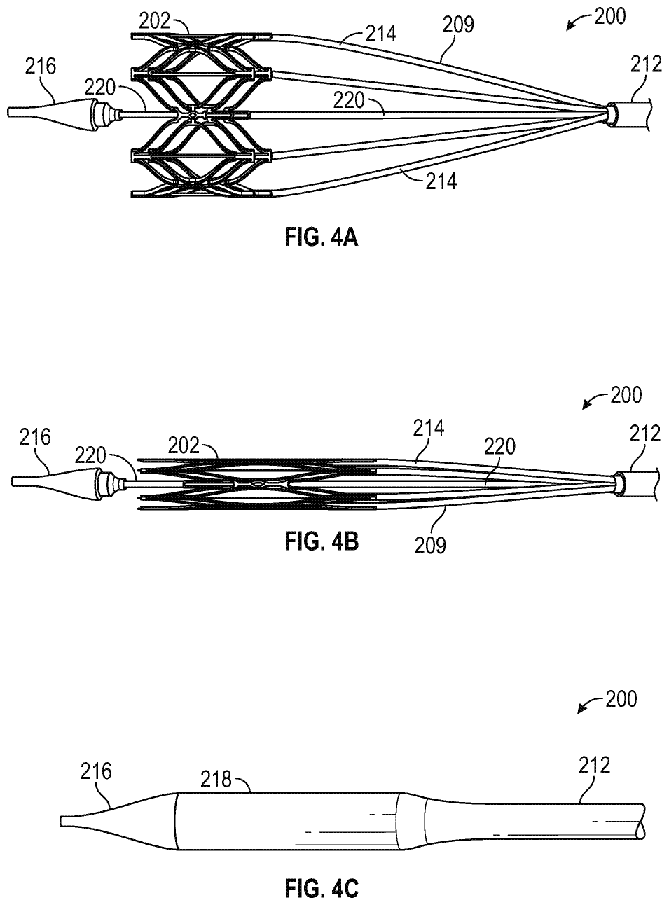

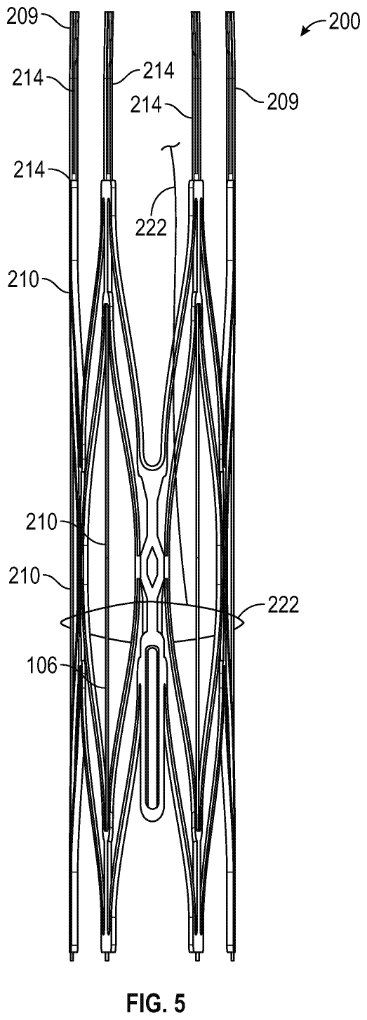

In some examples, illustrated in FIG. 4C, the restraining mechanism is a sheath 218 configured to surround and restrain the prosthetic heart valve in a radially compressed state. The sheath 218 can extend from the distal end of the outer shaft 212 of the delivery apparatus 200, or it can comprise a distal end portion of the shaft 212. When delivery apparatus 200 advances the prosthetic heart valve 202 to the implantation site, the sheath 218 can be retracted in the proximal direction (i.e., towards the handle of the delivery apparatus) to deploy the prosthetic heart valve 202. When the prosthetic heart valve 202 is deployed from the sheath, as shown in FIGS. 4A and 4B, which allows the prosthetic heart valve to self-expand to the partially radially expanded state (FIG. 4B) or to the fully radially expanded state (FIG. 4A).

In other examples, illustrated in FIG. 5, the restraining mechanism is an adjustable loop or lasso 222 circumferentially disposed around the exterior of the prosthetic heart valve 202. The adjustable loop is configured to allow the prosthetic heart valve to self-expand to the partially radially expanded state (FIG. 4B) or to the fully radially expanded state (FIG. 4A) by introducing slack in the loop 222, allowing it to increase in diameter.

The various prosthetic heart valves and delivery apparatus examples disclosed herein may further include a resistance-based expansion control mechanism according to various examples discussed below. Self-expanding or partially self-expanding prosthetic heart valves according to the present disclosure have a natural or innate self-expanding force resulting from the natural resiliency and elastic recovery of the materials and physical configuration of the prosthetic heart valve frame. These prosthetic heart valves also have a natural rate of self-expansion, which occurs when the prosthetic heart valve is allowed to freely expand solely under its own natural self-expanding force and in some circumstances any resistance to the self-expansion caused by the body of the patient. Expansion control mechanisms according to the present disclosure can reduce the rate of expansion of the self-expanding or partially self-expanding prosthetic heart valve disclosed herein by imposing a force that incompletely resists the self-expanding radial and/or axial forces of the prosthetic heart valve once released from any restraining mechanism.

In some examples, the resistance-based expansion control mechanism can comprise one or more friction elements or members disposed on a first component of the frame or delivery apparatus configured to move relative to a second component of the frame, such that the coefficient of friction between the friction element and the material of the frame is higher than the coefficient of friction between two opposing surfaces of the two component that do not have the friction elements. In other examples, the resistance-based expansion control mechanism can comprise a series of opposing mechanical features which are configured to move past each other in an axial direction at a controlled rate when under the forces of the self-expansion of the prosthetic heart valve. It is to be further understood that the resistance-based expansion control mechanism can include any other combination of materials and/or mechanical features which exert a force that incompletely opposes the self-expanding force of the prosthetic heart valves disclosed herein or which causes the self-expansion to occur at a lower rate than the natural rate of self-expansion.

FIGS. 6-9 illustrate a portion of a prosthetic heart valve frame 300. As shown in FIG. 6, the prosthetic heart valve frame 300 comprises a plurality of interconnecting struts 302 defining a plurality of cells of the prosthetic heart valve frame 300. The interconnecting struts 302 join at a distal end portion of the prosthetic heart valve frame 300 to form distal apex element 304 and join at a proximal end portion of the prosthetic heart valve frame 300 to form proximal apex elements 306. The apex elements 304, 306 are circumferentially aligned with one another and spaced apart axially. A prosthetic heart valve frame may be assembled from two or more such frame cells, such as three frame cells, four frame cells, five frame cells, six frame cells, seven frame cells, or eight frame cells. The frame portion of FIGS. 6-9 can represent a portion of the frames 102 of FIGS. 1-3. In other words, the features described in connection with FIGS. 6-9 can be incorporated in the frame of FIGS. 1-3.

As shown in FIG. 6, some examples of prosthetic heart valve frame 300, including those which are partially self-expanding, can include an actuation member 308 (which can be the same as actuation member 106). The actuation member 308 can be attached at a distal end to the distal apex element 304 and extend axially from the distal apex element 304 towards the proximal apex element 306. The actuation member 308 can additionally include a releasable attachment feature 310, disposed at a proximal end of the actuation member 308 and configured to releasably attach to a delivery actuator of a delivery apparatus, such as delivery system actuator 210 of delivery apparatus 200 previously discussed. The actuation member 308 is configured to exert an axial force on the distal apex element 304 to draw the distal apex element 304 towards the proximal apex element 306, while a corresponding support tube 214 applies an opposing force against the proximal apex element 306, thereby axially shortening prosthetic heart valve frame 300. While the actuation member 308 is shown as having a circular cross section in FIG. 6, it is to be understood that other geometric cross sections are possible, such as a rectangular cross section or an oval cross section, as two possible examples.

The actuation member 308 can be releasably attached to the distal end portion of a delivery actuator of a delivery apparatus such as delivery apparatus 200 in various ways. For example, as depicted in FIGS. 6, 8, and 9, the distal end portion of the actuation member 308 can comprise releasable attachment feature 310. The distal end portion of the delivery actuator, such as delivery system actuator 210 can include internal threads (or vice versa), thereby enabling the actuation member 308 and the delivery system actuator 210 to be threadably coupled together. In other examples, the actuation member 308 can comprise a lumen extending axially therethrough, and the delivery system actuator can comprise a wire or suture that extends through the lumen and is releasably secured to the outflow end portion of the frame. Various other types of releasable connections between the delivery system actuator and the actuation member can be used, such as interlocking releasable ridges disposed on the proximal end of the actuation member 308 and the distal end of the delivery system actuator.

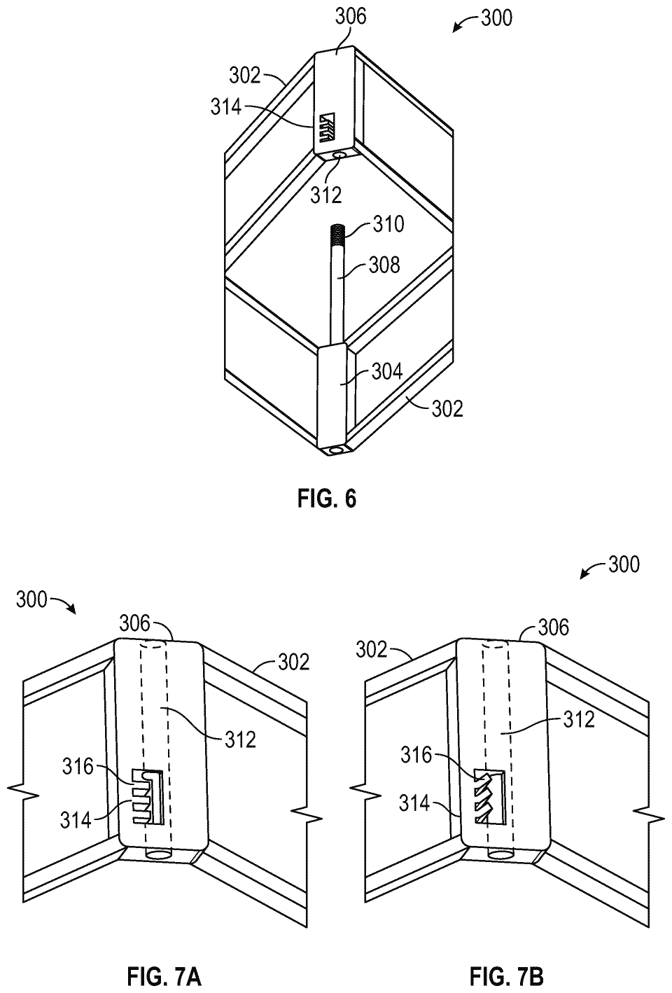

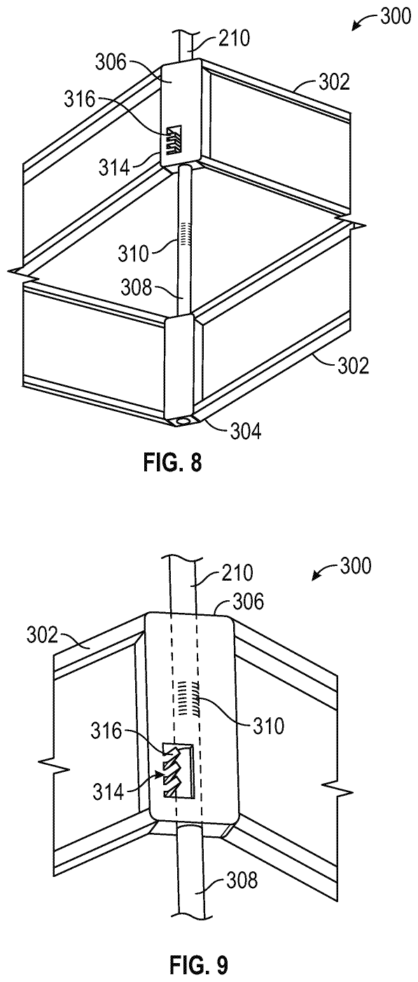

In some example prosthetic heart valve frames, proximal apex element 306 can include a channel 312 extending axially therethrough. As can be best seen in FIGS. 7A and 7B, the channel 312 can be configured to allow the delivery actuator of the delivery apparatus to pass through and connect to the actuation member 308. As shown in FIGS. 7A and 7B, the channel 312 can be a cylindrical bore extending through the length of the proximal apex element 306. In alternate examples, however, the channel 312 can be a bore with a different geometry, or an open rectangular groove in proximal apex element 306 through which the delivery actuator of the delivery apparatus may extend.

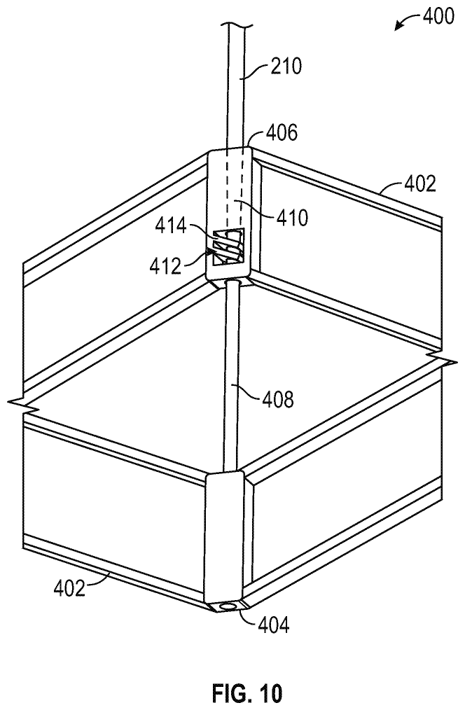

As illustrated in FIGS. 8 and 9, the channel 312 may additionally be configured to permit the actuation member 308 to move therein when the prosthetic heart valve frame 300 is moved from the radially compressed configuration to the partially radially expanded configuration, particularly in the case of partially self-expanding prosthetic heart valves which must be mechanically expanded from a partially-expanded configuration to the fully self-expanded configuration. As shown in FIG. 8, a delivery system actuator 210 and an actuation member 308 can be attached by releasable attachment feature 310. When the prosthetic heart valve is in a radially compressed condition, delivery system actuator can extend through the channel 312 in the proximal apex element 306, with the releasable attachment feature 310 located outside the channel 312 and distal to the proximal apex element 306. The actuator member 308 can be connected at its proximal end portion to delivery system actuator and at its distal end portion to distal apex element 304. As shown in FIG. 9, when the prosthetic heart valve self-expands, the connected actuator member 308 and delivery system actuator 210 can move axially in the proximal direction, drawing the proximal end portion of the actuator member 308 into the channel 312.

Once the prosthetic heart valve frame 300 is radially-expanded to a desired diameter, the delivery system actuator 210 of the delivery apparatus 200 can be released from the actuation member 308 of the prosthetic heart valve (e.g., by rotating the actuator 210 relative to the actuation member 308). The prosthetic valve can include a locking mechanism configured to retain the position of the actuation member 308 relative to the apex 306, thereby securing the prosthetic heart valve at the desired diameter, as further described below.

Returning to FIGS. 7A and 7B, the channel 312 can further include a locking mechanism 314. The locking mechanism 314 can be configured to permit the axial movement of the delivery system actuator 210 and the actuation member 308 in one direction (i.e. towards a proximal end portion of the prosthetic heart valve) and to prevent the axial movement of the delivery system actuator 210 and actuation member 308 in the opposite direction (i.e. towards a distal end portion of the prosthetic heart valve). As shown in FIG. 7A, the locking mechanism 314 can include one or more locking tabs 316. Although three locking tabs 316 are shown in FIG. 7A, the locking mechanism 314 can comprise fewer (e.g., 1-3) or more (e.g., 4 or more) than three locking tabs 316. The locking tabs 316 extend laterally and partially obstruct the channel 312. Accordingly, the locking tabs can engage the actuation member 308 of the prosthetic heart valve frame 300 when it is within the channel 312 and the frame is in the fully radially expanded configuration.

The locking tabs 316 of the locking mechanism 314 can be configured to deflect from a horizontal or unlocked position (FIG. 7A) to an angled or locked position (FIG. 7B). For example, the passage of the delivery system actuator 210 or the actuation member 308 of the prosthetic heart valve frame 300 over the locking tabs 316 can cause them to deflect into the angled configuration. When the locking mechanism 314 is in the locked position, the angled orientation of locking tabs 316 may allow the delivery system actuator 210 and/or actuation member 308 of the prosthetic heart valve frame 300 to move axially in the proximal direction, but may resist the axial motion of the delivery system actuator 210 and/or the actuation member 308 in the distal direction as shown in FIG. 7B.

Turning now to FIG. 10, which shows a portion of prosthetic heart valve frame 400, the locking mechanism can comprise a plurality locking tabs, each having a hole or aperture to allow the delivery actuator and/or the actuation member to pass therethrough. The prosthetic heart valve frame 400 can comprise a plurality of interconnecting struts 402 defining a plurality of cells of the prosthetic heart valve frame 400. The interconnecting struts 402 can join at a distal end portion of the prosthetic heart valve frame 400 to form a distal apex element 404 and join at a proximal end portion of the prosthetic heart valve frame to form proximal apex elements 406. The apex elements 404, 406 are circumferentially aligned with one another and spaced apart axially. A prosthetic heart valve frame may be assembled from two or more such frame cells, such as three frame cells, four frame cells, five frame cells, six frame cells, seven frame cells, or eight frame cells.

As shown in FIG. 10, prosthetic heart valve frame 400 can include an actuation member 408. The actuation member 408 can be attached at a distal end to the distal apex element 404 and extend axially from the distal apex element 404 towards the proximal apex element 406. Actuation member 408 is configured to exert an axial force on the distal apex element 404 to draw the distal apex element 404 towards the proximal apex element 406, thereby axially shortening prosthetic heart valve frame 400.

With continued reference to FIG. 10, the proximal apex element 406 can include a channel 410 extending axially therethrough. A locking mechanism 412 having a plurality of locking tabs 414 can be disposed in the channel 410. Locking tabs 414 may be configured to deflect from a horizontal, unlocked configuration (not shown) to a deflected, locked configuration, shown in FIG. 10. The locking tabs 414 can have a hole or aperture allowing the actuation member 408 and/or the delivery system actuator 210 to pass therethrough. This hole may have a diameter that is sufficient to permit the actuation member 408 and/or delivery system actuator to pass through when the locking tabs 414 are in an undeflected configuration, but small enough such that when the locking tabs 414 are in the deflected configuration, they bind against the actuation member 408 and prevent the axial motion thereof.

Further details regarding locking mechanisms that can be incorporated in any of the presently disclosed prosthetic valve are disclosed in U.S. Application No. 63/194,285, filed May 28, 2021, which is incorporated herein by reference. Such locking mechanisms may be useful, particularly with prosthetic heart valves according to the present disclosure that must be mechanically expanded to the fully expanded configuration, to prevent radial contraction of the prosthetic heart valve after it has been fully deployed. It is to be understood, however, that this feature may be omitted, particularly in fully self-expandable prosthetic heart valve frames.

While FIGS. 6-10 show only one frame portion, it is to be understood that a complete prosthetic heart valve frame is composed of multiple such frame portions. In some prosthetic heart valve examples, each frame portion of the frame may be identical to the examples shown in FIGS. 6-10, for example, each having an actuation member attached to each distal apex a channel passing through each proximal apex and configured to receive a delivery actuator of a delivery apparatus. In other prosthetic heart valve examples, however, there may be fewer actuation members than there are pairs of apices, for example, only half of the apices may have a corresponding actuation member, or the prosthetic heart valve may have one pair of apices without an actuator or two or more pairs of apices without an actuator. Additionally, some proximal apices can omit the channel. For example, only half of the proximal apices can have a channel, or the prosthetic heart valve may have one proximal apex without a channel or two or more proximal apices without a channel.

While FIGS. 6-10 all show a frame portion of a prosthetic heart valve having an actuation member, it is to be understood that in some examples, the actuation member may be omitted. For example, in the case of a fully self-expanding prosthetic heart valve, it may not be necessary to mechanically expand the prosthetic heart valve from the partially radially expanded configuration to the fully radially expanded configuration, and no number of actuation members 308 are needed.

As noted above, prosthetic heart valves according to the present disclosure can include resistance-based expansion control mechanisms disposed between a component of the frame of the prosthetic heart valve and another component, such as one of the delivery system actuators 210 of the delivery apparatus. The resistance-based expansion control mechanism can include materials with increased coefficients of friction relative to the materials used to form the frame and/or actuators 210, mechanical features, or a combination thereof.

In other examples, the resistance-based expansion control mechanism can be disposed between two components of the delivery apparatus that move relative to one another. In still other examples, the resistance-based expansion control mechanism can be disposed between two or more components of the prosthetic heart valve frame that move relative to one another.

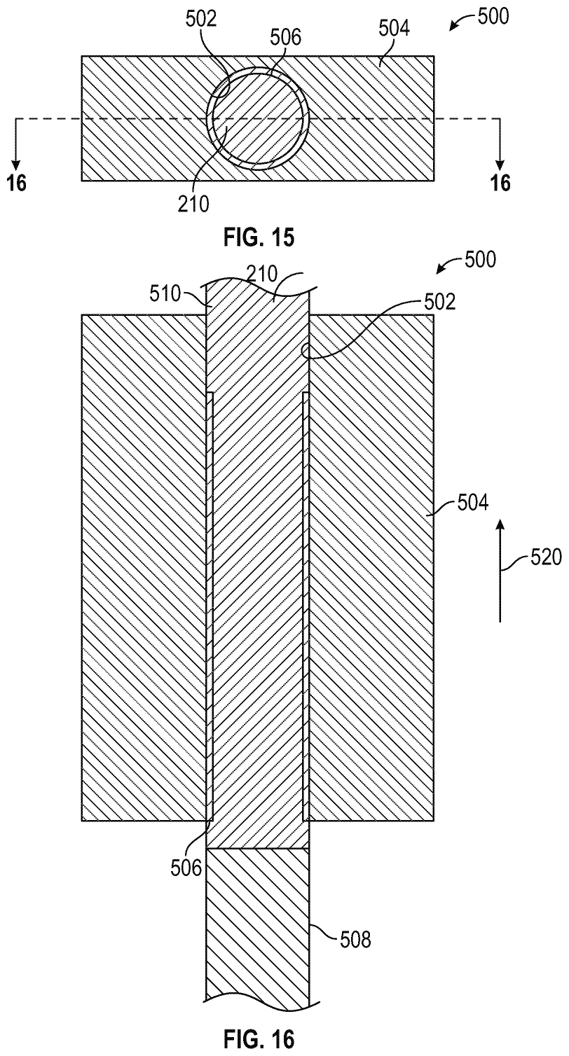

In one specific example, illustrated in FIGS. 15-18, a resistance-based expansion control system 500 can include a delivery system actuator, such as delivery system actuator 210 discussed above, passing through a channel 502 formed in a component 504 of a frame of a prosthetic valve. The delivery system actuator can include a resistance feature 506 (also referred to as a friction element or friction member). As best shown in FIGS. 15 and 16, the resistance feature 506 can be disposed on an external surface of the delivery system actuator 210. The delivery system actuator 210 and the resistance feature 506 can be configured to be movable axially within the channel 502, with the resistance feature 506 in slidable contact with an inner surface of the channel 502.

In some examples, the component 504 can be an apex of the frame. The apex can, in some examples, be a proximal apex, but in other examples may be a distal apex. In certain examples, the stationary component can be a proximal apex 120 of the frame 102, a proximal apex 306 of the frame 300, or a proximal apex 406 of the frame 400. In other examples, the component 504 can be a vertical strut of the frame that extends in the axial direction and interconnects with other struts, such as strut 118 of the frame 102. The delivery system actuator 210 can be releasably coupled to an actuator member 508 of the prosthetic valve. The actuator member 508 can be same as the actuator member 106, actuator member 308, or actuator member 408.

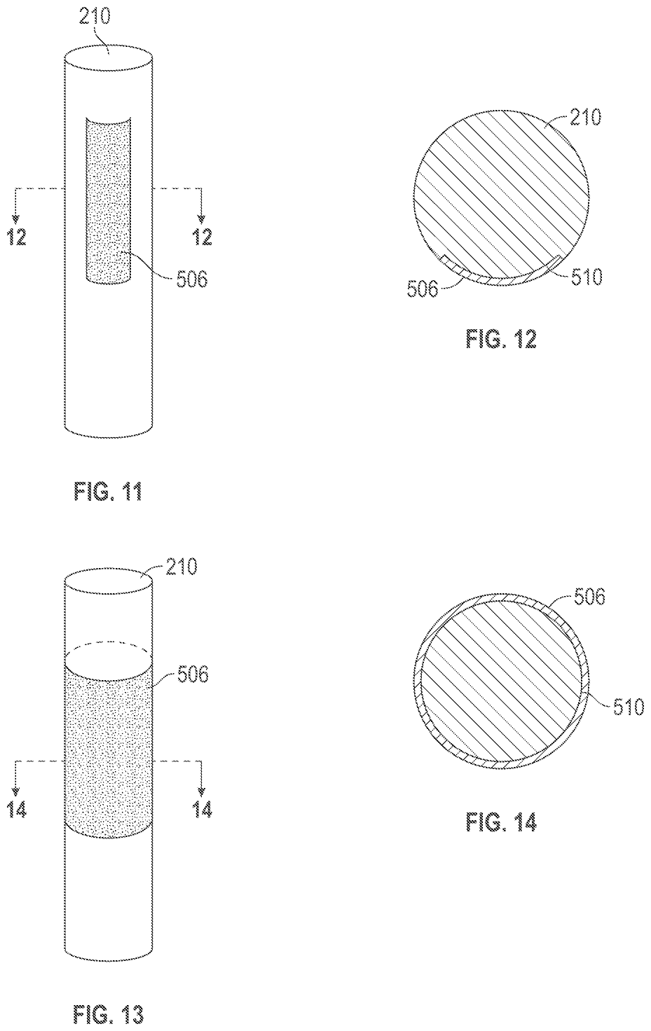

Turning now to FIGS. 11-14, the resistance feature 506 can be disposed on the exterior surface of the delivery system actuator 210. In some examples, illustrated in FIGS. 11 and 12, the resistance feature 506 can extend partially around the circumference of the delivery system actuator 210. In other examples, illustrated in FIGS. 13 and 14, the resistance feature can extend completely around the entire circumference of the delivery system actuator. In some examples, more than one resistance feature 506 can be included, such as two resistance features, three resistance features, or four resistance features, each of which can extend partially or fully around the circumference of the actuator. For example, an actuator 210 can include a plurality of resistance features 506 arrayed end-to-end or spaced apart from each other along the length of the actuator.

In the examples shown in FIGS. 11-14, the resistance feature 506 is disposed in a recess 510 formed in the outer surface of the actuator 210 and the outer diameter of the resistance feature 506 can be the same as the outer diameter of the actuator proximal and distal to the resistance feature. In other words, the resistance feature is flush with the outer surface of the actuator proximal and distal to the recess containing the resistance feature. In other examples, the actuator 210 can be formed without a recess for receiving the resistance feature such that the outer diameter of the resistance feature 506 is greater than the outer diameter of the actuator proximal and distal to the resistance feature.

The prosthetic heart valve frame can be made from a metal alloy. As previously discussed, the frame can be a plastically deformable alloy such as chromium cobalt alloy or stainless steel, or can be a shape memory alloy. In one particular example, the plastically deformable alloy can be a cobalt alloy having between 33 and 37 percent nickel content by weight, between 19 and 21 percent chromium content by weight, and between 9 and 10.5 percent molybdenum by weight (known also as M35N cobalt alloy). In another example, the shape metal alloy can be nitinol. Advantageously, nitinol is a shape memory alloy, which provides a strong elastic self-expanding force when the prosthetic heart valve is released from any retaining device. The delivery system actuator can be made from any of the same materials as the frame or may be a polymer material.

Resistance feature 506 can, in some examples, comprise a strip of high-friction material disposed on the external surface of the delivery system actuator. The strip of high-friction material can comprise any suitable material having a higher coefficient of friction with the interior surface of the channel 502 than the material of the delivery system actuator (e.g., typically a metal or metal alloy). In such examples, a first coefficient of friction exists between the delivery actuator and the interior surface of the channel 502 and a second coefficient of friction exists between the resistance feature 506 and the interior surface of the channel 502, wherein the second coefficient of friction is higher than the first coefficient of friction. In this way, the slidable contact between the high friction material of the resistance feature 506 can cause a greater friction force to resist the axial motion of the delivery system actuator 210 than would occur without the resistance feature. During the self-expansion of the prosthetic heart valve, this elevated friction force can thus partially oppose, but not wholly prevent, the expansion of the prosthetic heart valve, reducing the rate of prosthetic heart valve self-expansion and controlling any jumping of the prosthetic heart valve at the implantation site during the self-expansion stage of the implantation procedure.

The high friction material of resistance feature 506 can comprise a polymer. An ideal polymer for use in the resistance feature 506 is one with a high coefficient of friction between the polymer and the material of the frame (typically a metal or metal alloy) and a high degree of biocompatibility. In some examples, it may be advantageous for the polymer to be chemically modified to alter the coefficient of friction between the polymer and the material of the frame. In certain examples, the polymer material may be silicone, flouroelastomer, perflouroelastomer, ethylene propylene, nitrile rubber, or a combination thereof. Silicone offers many advantages for use in the resistance feature, including a high degree of biocompatibility, high biocompatibility, and favorable chemical and physical stability under the conditions of a prosthetic heart valve implantation procedure. While silicone is a particularly advantageous material for use in the resistance feature 506, it should be understood that any material which increases the coefficient of friction between the actuator and the frame and which has a high degree of biocompatibility may be suitable as well.

The resistance feature 506 can be separately formed, such as by molding, and then attached to the actuator 210, such as with a suitable adhesive. In other examples, the resistance feature 506 can be formed directly on the actuator 210, such as by dip coating the actuator 210 into a liquified polymer used for forming the resistance feature, or by spraying, molding, or extruding the polymer onto the actuator.

In other examples, the resistance feature 506 can be integrally formed in the delivery system actuator 210, as further described below. In still other examples, the resistance feature 506 can be a separate component such as an O-ring or gasket that is mounted on the delivery system actuator 210.

When the delivery system actuator moves relative to the prosthetic heart valve frame through the channel 502, a friction force will be generated based on the coefficient of dynamic friction between the material of the delivery system actuator and the material of the channel 502. The coefficient of dynamic friction between the channel 502 and the delivery system actuator 210 in the absence of the resistance feature 506 can be 0.6 or less, such as 0.5 or less, 0.4 or less, or 0.3 or less, such as when the actuator 210 and the component 504 are made from metal or a metal alloy. In certain specific examples, the coefficient of dynamic friction between the channel 502 and the resistance feature 506 can be at least 0.5, at least 0.6, at least 0.7, at least 0.8, at least 0.9, or at least 1.0.

In an alternative example, the resistance feature 506 can instead comprise a plurality of mechanical features configured to provide resistance to the axial motion of the delivery system actuator 210 within the channel 502. The plurality of mechanical features can include a first set of features disposed on the exterior surface of the delivery system actuator 210 and a corresponding second set of features disposed on the interior surface of the channel, and configured to slidably engage with the first set of features. The mechanical features may be configured to partially resist the axial motion of the delivery system actuator 210 within the channel while the prosthetic heart valve is self-expanding. In one example, the mechanical features may be a plurality of mutually engaging triangular or wedge-shaped teeth. In this way, the force required to pass one set of teeth over the other can provide resistance to the axial motion of the delivery system actuator 210 and the mutually engaging teeth can increase the contact area between the delivery system actuator 210 and the interior surface of channel 502, increasing the total frictional force between the delivery system actuator and the channel 502.

In another example, one of the first set or second set of mechanical features could comprise a plurality of projections, and the other of the first set or the second set of mechanical features could comprise a plurality of shallow depressions configured to receive or partially receive the plurality of projections. In this way, the additional force needed to cause the projections to come out of the shallow depressions can provide resistance to the axial motion of the delivery system actuator 210 and the interior surface of channel 502. Additionally, the depressions and projections may increase the total surface area in contact between the delivery system actuator 210 and the interior surface of channel 502.

It is to be understood that any other combination of mechanical features which impart a partial resistance to the axial motion of the delivery system actuator 210 within the channel 502 that resists, but does not completely stop the axial motion of the delivery system actuator 210 and the radial expansion of the prosthetic heart valve may be used. It is also to be appreciated that a resistance feature 506 comprising a plurality of mechanical features may additionally include any of the high friction materials discussed above.

During a prosthetic heart valve implantation procedure, the prosthetic heart valve can radially self-expand from the radially compressed state to a partially radially expanded state under the resilient and/or elastic forces of the prosthetic heart valve frame. This can be accomplished by deploying the prosthetic valve from a delivery sheath 218 or increasing the diameter of an adjustable loop 222 encircling the prosthetic valve. The radial expansion of the frame shortens the frame axially, causing the delivery system actuator 210 to move axially in the proximal direction within the channel 502 (in the direction of arrow 520 in FIG. 16). As depicted in FIG. 16, the resistance feature 506 disposed on the delivery system actuator 210 remains in slidable contact with the internal surface of the channel 502 while the valve is self-expanding, and imparts a force on the delivery system actuator that partially resists, but does not fully stop, the axial motion of the delivery system actuator 210.

When the axial motion of the delivery system actuator 210 is resisted, the rate of expansion of the prosthetic heart valve is be reduced from a natural expansion rate to a controlled expansion rate that is lower than the natural expansion rate. In this way, any tendency of the prosthetic heart valve to jump or become misaligned during self-expansion can be avoided. In certain examples, the resistance feature 506 is sized and configured such that it is in contact with the surface of the channel 502 from the radially compressed delivery state (such as shown in FIGS. 2 C and 4B) to the partially expanded state (such as shown in FIG. 2B). In this manner, the contact between the resistance feature 506 and the surface of the channel 502 can reduce the rate of expansion of the prosthetic valve throughout the entire self-expansion range of the prosthetic valve.

In certain examples, the upper limit of the self-expansion range (the diameter of the prosthetic valve when it stops expanding under the resiliency of the frame) is selected to be less than the diameter of the annulus of the native heart valve in which the prosthetic valve is to be implanted. Thus, in such examples, when prosthetic valve reaches the upper limit of the self-expansion range, the prosthetic valve does not contact the surrounding native annulus. The prosthetic valve can be further expanded by mechanical actuation to its final desired working diameter, as further described below.

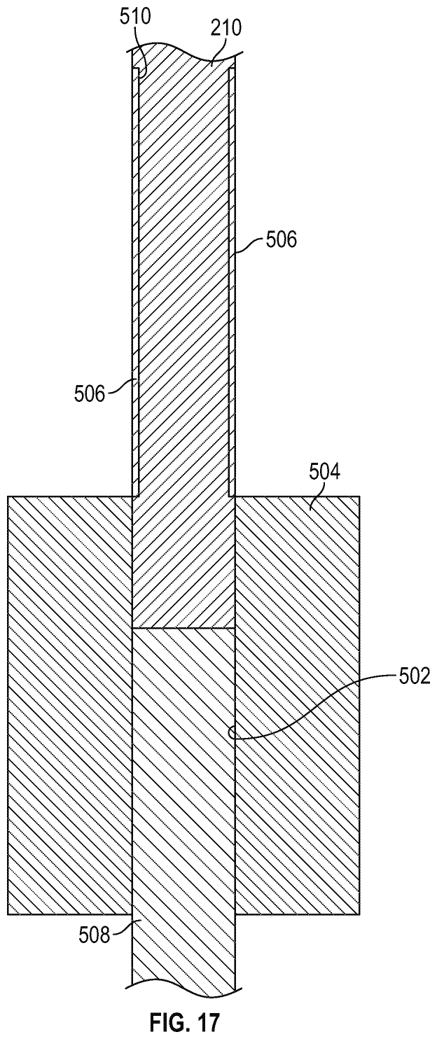

FIG. 17 depicts the location of the resistance feature 506 relative to the component 504 at the upper limit of the self-expansion range. As shown, the resistance based expansion control mechanism can be configured such that, when the prosthetic heart valve reaches the upper limit of the self-expansion range, the resistance feature 506 comes fully out of engagement with the inner surface of the channel 502. In this way, the resistance feature 506 can be prevented from providing any resistance to the axial motion of the delivery system actuator 210 during subsequent expansion from the partially expanded state to the fully expanded state. As the prosthetic valve self-expands, the delivery system actuator 210 can pull the actuator member 508 of the prosthetic valve into the channel 502, as shown in FIG. 17.

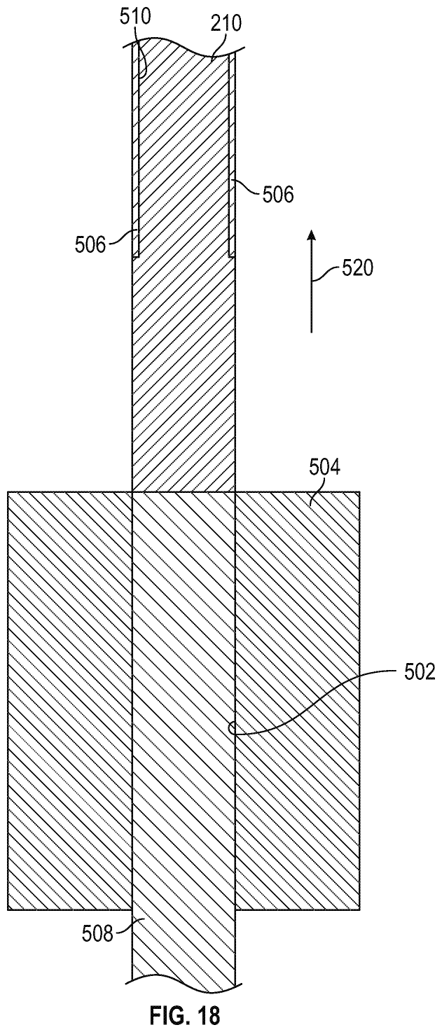

Turning now to FIG. 18, after the prosthetic heart valve has self-expanded to the partially radially expanded condition, the delivery system actuator 210 can be actuated by the user to pull the actuation member 508 proximally (in the direction of arrow 520) to further radially expand the prosthetic valve. This radially expands the prosthetic heart valve (such as prosthetic heart valve 100) to the fully radially expanded condition, and draws the delivery system actuator 210 fully out of the channel 502. When the delivery system actuator 210 is drawn out of the channel, the actuation member 508 can be axially pulled in the proximal direction into the channel.

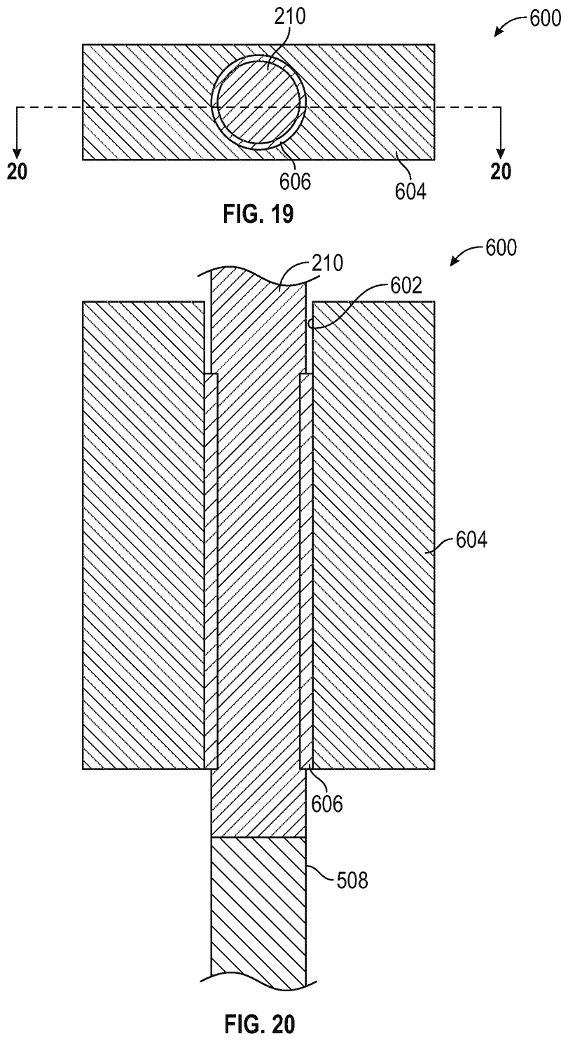

FIGS. 19 and 20 show an alternative example of a resistance-based expansion control system. As shown in FIGS. 19 and 20, resistance-based expansion control system 600 can include a delivery system actuator, such as delivery system actuator 210 discussed above, passing through a channel 602 formed in a frame component 604 of the frame of a prosthetic valve. The frame component 604 can be, for example, an apex of the frame (e.g., a proximal apex 120 of the frame 102, a proximal apex 306 of the frame 300, or a proximal apex 406 of the frame 400) or a strut of the frame. The resistance-based expansion control system also includes one or more resistance features 606 disposed on an exterior surface of the delivery system actuator 210. Resistance-based expansion control system 600 functions in substantially the same way as resistance-based control system 500 discussed above, except that the external surface of the resistance feature 606 extends radially past the external surface of the delivery system actuator 210. Stated differently, the resistance feature 606 has an outer diameter greater than the outer diameter of the actuator 210. In this way, only resistance feature 606 and not the delivery system actuator 210 can be in slidable contact with the interior surface of the channel 602.

Resistance feature 606 can be made of any of the materials previously discussed in connection with resistance feature 506, including biocompatible polymers or elastomers, particularly silicone, flouroelastomer, perflouroelastomer, ethylene propylene, nitrile rubber, or a combination thereof. When the resistance feature 606 comprises a high friction material, it can be formed on the delivery system actuator 210 or can be a separate component such as an O-ring or gasket that is set in a groove in the delivery system actuator 210, as previously described. Resistance feature 606 can also include any of the plurality of mechanical features discussed above that would be suitable for resistance feature 506. Resistance feature 606 can further incorporate any combination of suitable mechanical features and materials as previously discussed.

Advantageously, resistance-based control system 600 can allow controlled expansion of the prosthetic heart valve during the self-expansion phase, while minimizing or preventing contact between the delivery system actuator 210 and the interior of the channel 602 when the self-expansion of the prosthetic heart valve has been completed. In this way, resistance to any mechanical expansion of the prosthetic heart valve that follows the self-expansion of the prosthetic heart valve may be minimized or prevented.

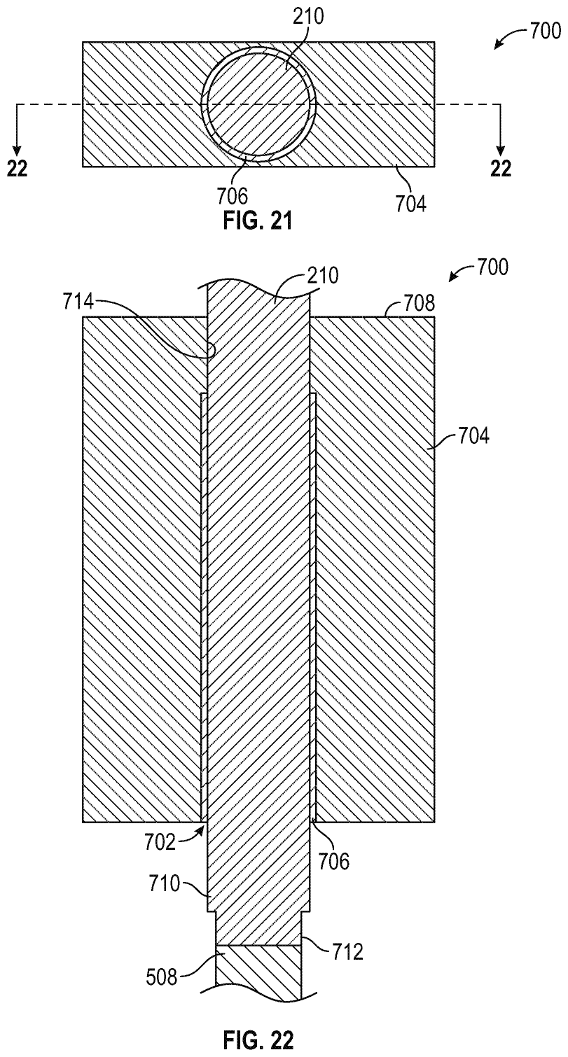

In another alternative example, a resistance-based expansion control system can have the resistance feature attached to the interior surface of the channel, rather than on the exterior surface of the delivery system actuator. As shown in FIGS. 21 and 22, a resistance-based expansion control system 700 can include a delivery system actuator, such as delivery system actuator 210 discussed above, passing through a channel 702 formed in a frame component 704 of a frame of a prosthetic valve. The frame component 704 can be, for example, an apex of the frame (e.g., a proximal apex 120 of the frame 102, a proximal apex 306 of the frame 300, or a proximal apex 406 of the frame 400) or a strut of the frame. The resistance-based expansion control system also includes one or more resistance features 706 disposed on the interior surface of the channel 702.

In some examples, such as illustrated in FIG. 22, the delivery system actuator 210 can have a proximal portion 710 with a first diameter and a distal portion 712 with a second diameter. In one example, the first diameter is greater than the second diameter. When the prosthetic heart valve is in the radially compressed configuration or is self-expanding, the proximal portion 710 of the delivery system actuator 210 can be within channel 702 and in contact with the interior surface of the channel 702. As the prosthetic heart valve self-expands, the proximal portion 710 of the delivery system actuator 210 slidably engages the resistance feature 706 disposed on the internal surface of channel 702, imparting a force that partially resists the axial motion of the delivery system actuator 210 and thereby controls the rate of self-expansion of the prosthetic heart valve. As the prosthetic heart valve reaches the partially self-expanded state and the self-expansion is completed, the proximal portion 710 of the delivery system actuator 210 may pass axially out of the channel 702 and thus out of contact with the resistance feature 706. The distal portion 712 of the delivery system actuator 210 can then enter the channel, but because the distal portion 712 of the delivery system actuator 210 has a smaller circumference than the proximal portion of the delivery system actuator, the external surface of the distal portion of the delivery system can avoid contact with the resistance feature 706 so as to prevent or minimize any sliding friction between the channel 702 and the actuator 210 during subsequent expansion of the prosthetic valve following the self-expansion phase.

In another example, the portion of the actuator 210 that extends through the channel 702 can have a constant diameter. In this example, the resistance feature 706 can be sized such that the distal end portion of the delivery system actuator 210 engages the resistance feature during the self-expansion phase and then fully disengages the resistance feature 706 when the self-expansion of the prosthetic heart valve is completed. In examples in which the prosthetic heart valve is partially mechanically-expandable, the resistance feature may stop short of a proximal end 708 of the stationary component 704, defining a distal portion 714 of the channel 702 having no resistance features. In examples in which the prosthetic heart valve is fully self-expanding, the resistance feature may extend all the way to the proximal end 708 of the stationary component 704, such that the delivery system actuator 210 is in contact with the resistance feature 706 for as long as it inside the channel 702, and only comes fully out of contact with the resistance feature 706 when it fully leaves the channel 702.

Resistance feature 706 can be made of the same high-friction materials previously discussed in connection with resistance feature 506, including biocompatible polymers or elastomers, particularly silicone, flouroelastomer, perflouroelastomer, ethylene propylene, nitrile rubber, or a combination thereof. When the resistance feature 706 comprises a high friction material, it can be formed on the internal surface of the channel 702 or can be a separate component such as an O-ring or gasket that is set in a groove in the channel 702, as previously described. Resistance feature 706 can also include any of the plurality of mechanical features discussed above that would be suitable for resistance feature 506. Resistance feature 706 can further incorporate any combination of suitable mechanical features and materials as previously discussed.

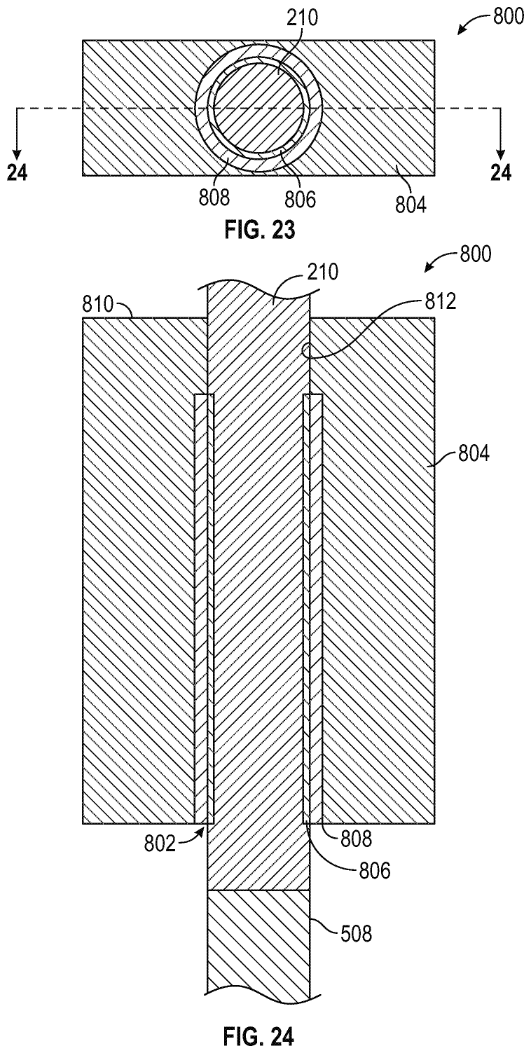

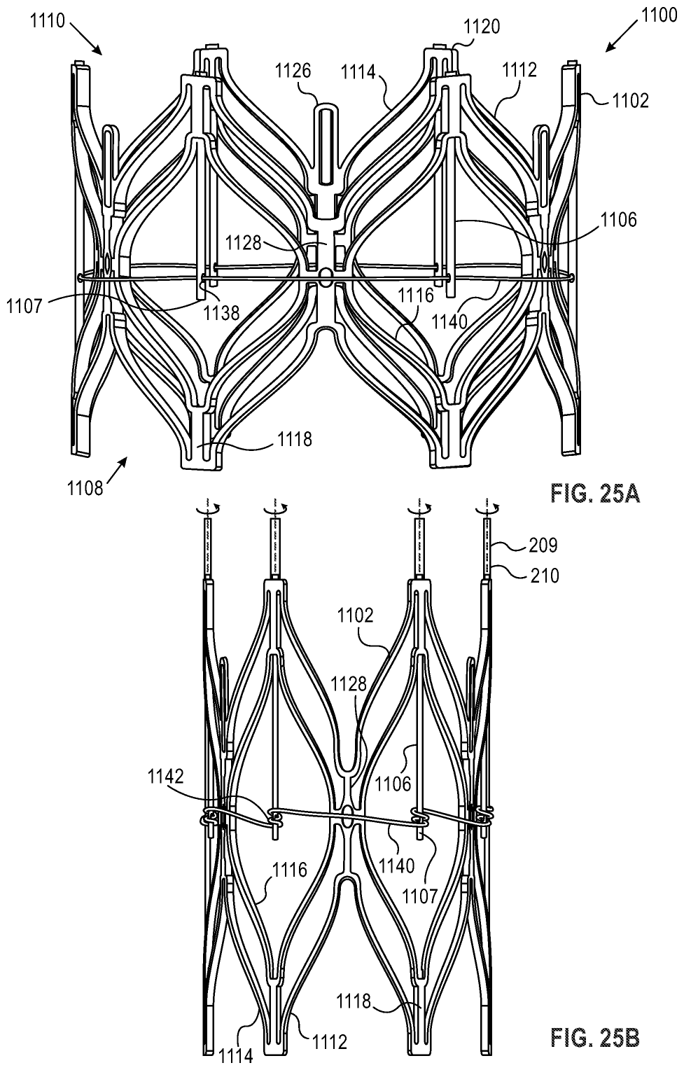

In another alternative example, a resistance-based expansion control system can have a resistance feature disposed on and attached to an interior surface of the channel and an external surface of the delivery system actuator. As shown in FIGS. 23 and 24, resistance-based expansion control system 800 can include a delivery system actuator, such as delivery system actuator 210 discussed above, passing through a channel 802 formed in a frame component 804 of a frame of a prosthetic valve. The frame component 804 can be, for example, an apex of the frame (e.g., a proximal apex 120 of the frame 102, a proximal apex 306 of the frame 300, or a proximal apex 406 of the frame 400) or a strut of the frame. A first resistance feature 806 can be attached to an external surface of the delivery system actuator 210 and a second resistance feature 808 can be attached to an internal surface of the channel 802.

Resistance-based expansion control system 800 functions in substantially the same way as resistance-based control system 500 discussed above, except that the first resistance feature 806 is configured to slidably engage with the second resistance feature 808. When the prosthetic heart valve is in the radially compressed configuration, first resistance feature 806 and second resistance feature 808 are in contact with each other within the channel 802. When the prosthetic heart valve self-expands to at least the partially radially expanded configuration and delivery system actuator 210 moves axially within the channel 802 towards the proximal end of the prosthetic heart valve, the first resistance feature 806 slides against the second resistance feature 808.

In partially mechanically expandable prosthetic heart valves, the second resistance feature 808 can stop short of a proximal end 810 of the frame component 804, defining a proximal end portion 812 of the channel having no resistance feature. In this way, when the prosthetic heart valve reaches the partially self-expanded configuration and self-expansion ceases, the delivery system actuator 210 will have moved far enough axially through the channel in a proximal direction that the first resistance feature 806 can come fully out of contact with the second resistance feature, the proximal end portion 812 of the channel 802, or both. This will allow the first resistance feature 806 and second resistance feature 808 to control the rate of expansion of the prosthetic heart valve when it is radially self-expanding, but provide no resistance during the mechanical expansion of the prosthetic heart valve from the partially radially-expanded configuration to the fully radially-expanded configuration.

In fully self-expandable prosthetic heart valves, the second resistance feature 808 can extend all the way to the proximal end 810 of the frame component 804, and proximal end portion 812 can be omitted. In this way, when the first resistance feature and the second resistance feature may remain in contact for the full range of self-expansion of the prosthetic heart valve, coming out of engagement only when the prosthetic heart valve has fully radially self-expanded. This will allow the first resistance feature 806 and the second resistance feature 808 to control the rate of expansion over the full range of radial expansion of the prosthetic heart valve.

Advantageously, utilizing two resistance features permits potentially a higher coefficient of friction to partially oppose the radial expansion of the prosthetic heart valve, as the coefficient of friction between two high friction materials may be higher than the coefficient of friction between the high friction material and the base material of the frame (e.g., nitinol, stainless steel, or cobalt-chromium alloy). In some examples, a locking mechanism, such as locking mechanism 314 previously discussed, can be included in the channel. Locking mechanism 314 can be configured to engage with actuation member 308, 408 or 508 when the prosthetic heart valve has been expanded to the fully radially expanded configuration, and to prevent the actuation member 308, 408 or 508 from axially moving in the distal direction, the proximal direction, or either direction. In this way, the prosthetic heart valve may be prevented from further axial expansion or contraction after it has been implanted.

In all such examples, the resistance-based expansion control mechanism can be configured to engage only when the prosthetic heart valve is self-expanding, and to come out of engagement when the self-expansion of the prosthetic heart valve is complete. In this way, the resistance-based control mechanism can provide resistance only to oppose the natural self-expansion of the prosthetic heart valve frame, and to provide no resistance to any subsequent mechanical expansion of the prosthetic heart valve frame.

In examples in which the prosthetic heart valve frame is fully self-expanding, the self-expansion of the prosthetic heart valve can be sufficient to cause the delivery system actuator 210 to fully exit the channel of the frame component (e.g., channel 502). In such examples, the locking mechanism may be omitted as unnecessary. The frame of the prosthetic valve can still include a member 508 that moves axially through a channel of a frame component (e.g., channel 502), but does not need to be used as an actuator to apply a proximally directed force to expand the frame. Instead, the member 508 and/or the channel can include resistance features of any of the configurations shown in FIGS. 11-24 to control the rate of self-expansion of the prosthetic for part of or the entire range of self-expansion of the prosthetic valve. In the case of fully self-expanding prosthetic heart valves, the resistance feature 506 may extend to a distal end of the delivery system actuator 210, so that the resistance feature 506 will remain engaged until the delivery system actuator has fully exited the channel 502, which may in some examples occur only when the fully self-expanding prosthetic heart valve has fully self-expanded. In other examples, the delivery system actuator 210 may be configured to exit the channel 502 before the fully self-expanding prosthetic heart valve has fully self-expanded. In other examples, the resistance feature 506 can extend only part of the way towards the distal end of the delivery system actuator so that, even if the delivery system actuator 210 fully exits the channel 502 only when the prosthetic heart valve reaches the fully-expanded configuration, the resistance feature 506 will become disengaged from the interior surface of the channel 502 before that point.

According to several alternative examples, the component 504, 604, 704, or 804 can be a component of the delivery apparatus, such as a support tube disposed around the delivery system actuator (for example, support tube 214). In such examples, the channel 502, 602, 702, or 802 can be a bore or channel in the support tube 214 configured to permit the delivery system actuator 210 to move axially therethrough. In this way, the resistance-based expansion control mechanism can be wholly incorporated in the delivery apparatus, and requires no direct engagement with the components of the prosthetic heart valve frame. Thus, in any of the configurations of FIGS. 15-24, the component 504, 604, 704, or 804 can represent a support tube 214 of the delivery apparatus. At least one pair of an actuator 210 and a support tube 214 can include one or more of resistance features 506, 606, 706, 806 and/or 808. In certain examples, each pair of an actuator 210 and a support tube 214 of the delivery apparatus can include one or more of resistance features 506, 606, 706, 806 and/or 808.

In operation, such a resistance-based control mechanism disposed between two components of the delivery device may function in a similar fashion to a resistance-based control mechanism disposed between the delivery system actuator and a component of the prosthetic heart valve frame. When the self-expanding prosthetic heart valve is released from a restraining mechanism, the elastic or resilient forces of the frame cause the frame to radially self-expand to a partially expanded configuration. The radial self-expansion of the prosthetic heart valve frame causes the frame to axially foreshorten, and causes the delivery system actuators 210 attached to the frame to move axially through the channels in the support tubes 214 in the proximal direction. This causes the resistance features disposed between the delivery system actuators and the support tubes to engage with an opposing surface to impart a resistance force that partially resists, but does not completely stop, the axial motion of the delivery system actuators through the channel. This in turn, controls the rate of radial expansion of the prosthetic heart valve and mitigates the effects of jumping or sudden, uncontrolled expansion of the prosthetic heart valve within the vasculature of the patient.

In resistance-based expansion control mechanisms incorporated wholly into a delivery device, such as delivery apparatus 200, the resistance feature can be disposed on the exterior of the actuator and configured to be in slidable contact with an interior surface of the support tube 214. Returning to FIGS. 14 through 17, stationary component 504 can be the support tube 214 of a delivery device. The system 500 can comprise a channel 502 through the stationary component 504, a delivery system actuator such as delivery system actuator 210, and a resistance feature 506 disposed between an external surface of the delivery system actuator and an interior surface of the channel 502.

According to one example shown in FIG. 15, the resistance feature, such as resistance feature 506 can sit in a recess or groove in the delivery system actuator 210, and can be configured to be flush with the exterior surface of the delivery system actuator 210. According to another example, shown in FIG. 19, the resistance feature, such as resistance feature 506, can have an outer surface that extends radially past the surface of the delivery system actuator 210, such that only the resistance feature, such as resistance feature 506, is in contact with the interior surface of the channel, such as channel 502 In another example, such as that illustrated in FIG. 21, the resistance feature, such as resistance feature 706, can be disposed secured the interior surface of the channel of the support tube 214, such as channel 702. In yet another example, such as illustrated in FIG. 23, the resistance-based expansion control mechanism can include both a resistance feature such as resistance feature 706 secured to the delivery system actuator 210 and a resistance feature such as resistance feature 706 secured to the interior surface of the channel of the support tube 214, such as channel 702.

In any of the examples just discussed where the component 504, 604, 704, or 804 is a support tube 214 or another component of the delivery apparatus, the resistance feature can comprise any of the materials discussed above in connection with the examples where the component 504, 604, 704, or 804 is a component of the frame.

In an alternative example, the resistance feature (e.g., 506, 606, 706, 806, 808) can instead comprise a plurality of mechanical features configured to provide resistance to the axial motion of the delivery system actuator (e.g., delivery system actuator 210) within the channel (e.g., channel 502, 602, 702, or 802) of the support tube 214. The plurality of mechanical features can include a first set of features disposed on the exterior surface of the delivery system actuator and a corresponding second set of features disposed on the interior surface of the channel, and configured to slidably interconnect with the first set of features. The mechanical features may be configured to partially resist the axial motion of the delivery system actuator within the channel while the prosthetic heart valve is self-expanding. In one example, the mechanical features may be a plurality of mutually engaging triangular or wedge-shaped teeth. In this way, the force required to pass one set of teeth over the other can provide resistance to the axial motion of the delivery system actuator and the mutually engaging teeth can increase the contact area between the delivery system actuator and the interior surface of channel, increasing the total frictional force between the delivery system actuator and the channel.

In another example, one of the first set or second set of mechanical features could comprise a plurality of projections, and the other of the first set or the second set of mechanical features could comprise a plurality of shallow depressions configured to receive or partially receive the plurality of projections. In this way, the additional force needed to cause the projections to come out of the shallow depressions can provide resistance to the axial motion of the delivery system actuator (e.g., delivery system actuator 210) and the interior surface of channel (e.g., channel 502, 602, 702, or 802) of the support tube 214. Additionally, the depressions and projections may increase the total surface area in contact between the delivery system actuator and the interior surface of channel, in turn increasing the total frictional force even without increasing the coefficient of friction between the actuator and the interior surface of the channel.

FIGS. 25A-25B illustrate another example of a prosthetic heart valve 1100, which includes a frame 1102 that can be structurally similar to and function in a similar manner as the frame 102 of prosthetic heart valve 100, with like number referring to like components. The frame 1102 extends from a first end 1108 to a second end 1110, which can be the inflow end and outflow end, respectively. The frame 1102 includes a plurality of interconnected struts 1112 that define a plurality of cells, such as primary cells 1114 and secondary cells 1116 in the illustrated example. The primary cells 1114 and secondary cells 1116 are interconnected at their ends by vertical struts 1118 that form apices 1120 at the first end 1108 and the second end 1110 of the frame 1102. The prosthetic valve 1100 further includes a valvular structure which can be structurally similar to and function in a similar manner as the valvular structure 104 of prosthetic heart valve 100 (not shown in FIGS. 25A-25B for clarity). The frame 1102 can include a plurality of vertical struts 1128 disposed circumferentially between adjacent pairs of the primary cells 1114. The frame can further include a plurality of leaflet attachment structures, such as commissure windows 1126 in the illustrated example, extending from at least some of the vertical struts 1128.

The prosthetic valve 1100 further comprises actuation members 1106 (e.g., six actuators in the illustrated example) which are coupled to the frame 1102 and are configured to adjust expansion of the frame 1102 from a compressed or a partially radially-expanded state (e.g., FIG. 25B) to a plurality of configurations including one or more functional or expanded configurations (e.g., FIG. 25A). In some implementations, prosthetic valve 1100 can include a fully self-expandable frame 1102. The actuation members 1106 of the prosthetic heart valve 1100 are mounted to and spaced circumferentially around the frame 1102. In the illustrated example, the prosthetic heart valve 1100 comprises six actuation members 1106. In other examples, the prosthetic heart valve can comprise fewer or more than six actuation members (e.g., 1-5 or 7-15).

The actuation members 1106 can be rotatably coupled to the frame 1102, such that they can rotate about their axes while remaining immovable in the circumferential direction with respect to the frame 1102. For example, the actuation members 1106 can be formed as relatively rigid rod members axially extending through channels formed in vertical struts 1118 extending from the apices 1120 at the outflow end 1110 of the frame 1102. The actuation members 1106 further comprise actuation member apertures 1138 extending through the thickness of the actuation members 1106 in the circumferential direction. The actuation member apertures 1138 can be positioned within the secondary cells 1116 as shown in the illustrated example. The actuation members 1106 can terminate at free-ended terminal ends 1107, as illustrated. However, in other examples, the terminal ends 1107 can be rotatably attached to the frame 1102, for example by extending toward, and being rotatably coupled to, the opposite vertical struts 1118 extending from the apices 1120 at the inflow end 1108 of the frame 1102.

The prosthetic heart valve 1100 further comprises a flexible tension member 1140 extending through the actuation member apertures 1138 and encircling the frame 1102. As mentioned, the prosthetic heart valve 1100 can be configured to radially self-expand from a radially compressed state to a partially or fully expanded state under resilient and/or elastic forces of the prosthetic heart valve frame 1102. The flexible tension member 1140 can be utilized to retain the frame 1102 in a compressed or partially expanded diameter, while increasing the diameter of the flexible tension member 1140 can allow the frame 1102 to expand further, potentially up to a fully expanded or functional diameter. Otherwise stated, the flexible tension member 1140 can apply a radially inwardly directed force to the frame 1102 that can be gradually lessened such that the frame 1102 can expand at a controlled rate (e.g., a rate selected by the physician) to a selected diameter.

Each actuation member 1106 can be coupled to an actuator assembly 209 and/or a delivery system actuator 210 of the delivery apparatus 200 previously discussed. In some implementations, each actuation member 1106 can include a releasable attachment feature, disposed at a proximal end thereof (not shown), configured to releasably attach to an actuator assembly 209 and/or a delivery system actuator 210 of the delivery apparatus 200. The delivery system actuators 210 can be configured to apply rotational movement to the corresponding actuation members 1106 they are coupled to, in either direction (i.e., clockwise or counterclockwise), to compress or allow self-expansion of the prosthetic heart valve 1100.

The flexible tension member 1140 serves as a restraining mechanism, and can comprise a wire, string, and/or cable. As mentioned, the flexible tension member 1140 extends circumferentially around the frame 1102, and through the actuation member apertures 1138. In some examples, the flexible tension member 1140 can be weaved through cells 1114, 1116 of the frame 1102, for example by weaving it in an in-and-out pattern along struts 1112 and/or vertical struts 1128 of the frame 1102. In other examples, the flexible tension member 1140 can be disposed entirely around the frame 1102, except for the portions passing through actuation member apertures 1138. In some examples, the frame 1102 can comprise a plurality of additional eyelets or apertures through which portions of the flexible tension member 1140 can be threaded.

The flexible tension member 1140 can extend at least partially around the circumference of the frame 1102. In the illustrated example, the flexible tension member 1140 extends around frame 1102 such that it forms a single loop encircling the frame 1102. In other examples, the flexible tension member 1140 can be disposed such that it extends around the frame 1102 in a continuous helical manner defining a plurality of loops. In still other examples, the flexible tension member 1140 can be disposed such that it spans a distance less than the full circumference of the frame 1102. In some cases, the flexible tension member 1140 can be attached to the frame 1102 at one or more locations, for example by forming knots around struts or posts of the frame 1102 at one or two ends of the flexible tension member 1140.

FIG. 25A shows the frame 1102 in a fully radially expanded state. In order to compress it to a partially radially-expanded state as shown in FIG. 25B, or to a fully radially-compressed or crimped state (similar to the state of frame 102 in FIG. 2C, for example), the actuator assemblies 209 and/or a delivery system actuators 210 of the delivery apparatus 200 can be rotated in a second rotational direction (e.g., counterclockwise in the illustration of FIG. 25B), so as to impart rotational movement of the actuation members 1106 in the same rotational direction therewith. As the actuation members 1106 rotate around their respective central axes, local loops 1142 of the flexible tension member 1140 are formed around the respective actuation members 1106, thereby tensioning the flexible tension member 1140 and shortening the lengths of the adjustable loop sections circumferentially extending between adjacent actuation members 1106, in a manner that will reduce the diameter defined by the flexible tension member 1140 and compress the frame 1102 therewith, for example, from the radially expanded state of FIG. 25A to the partially radially-expanded state as shown in FIG. 25B, wherein continued rotation will further compress the frame 1102 to the radially-compressed or crimped state (similar to the state of frame 102 in FIG. 2C, for example).

As shown in the illustrated example, the actuation member apertures 1138 can be positioned approximately halfway between the inflow end 1108 and the outflow end 1110 of the prosthetic valve 1100 so that the restraining force of the flexible tension member 1140, when restricting the frame 1102 to a fully or partially compressed diameter, is equally distributed along the length of the frame. In alternative embodiments, the actuation member apertures 1138 can be closer to the inflow end 1108 or closer to the outflow end 1110 of the prosthetic valve 1100, which can result in the prosthetic heart valve 1100 assuming a substantially tapered V-shaped or A-shaped configuration (having one end thereof narrower than the opposite end) upon expansion.

When the prosthetic valve 1100 is deployed from the delivery sheath 218, it can be allowed to self-expand by rotating the actuator assemblies 209 and/or a delivery system actuators 210 of the delivery apparatus 200 in the first rotational direction (e.g. clockwise), causing the actuation members 1106 to rotate therewith, unraveling the local loops 1142 and allowing the flexible tension member 1140, and the frame 1102 encircled thereby, to increase in diameter, for example up to the radially expanded state shown in FIG. 25A. In some applications, the prosthetic heart valve 1100 can be allowed to fully expand by unraveling all local loops 1142. In alternative implementations, the prosthetic heart valve 1100 can be expanded to a selected expanded diameter that can be less than the full diameter, by increasing the diameter of the flexible tension member 1140 to allow the frame 1102 to partially expand therewith, but still restrict further expansion. In this manner, the extent to which the actuation members 1106 can serve as an expansion diameter control mechanism. After partial or full expansion is achieved, the actuator assemblies 209 and/or delivery system actuators 210 of the delivery apparatus 200 can be disengaged from the actuation members 1106, allowing retrieval of the delivery apparatus 200 from the patient's body.

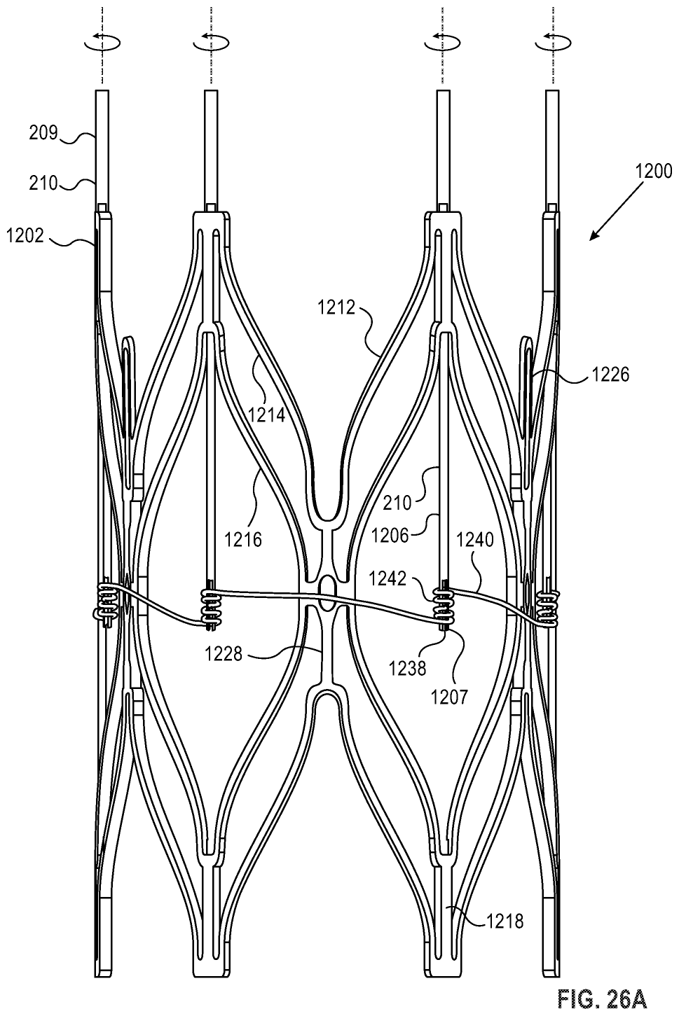

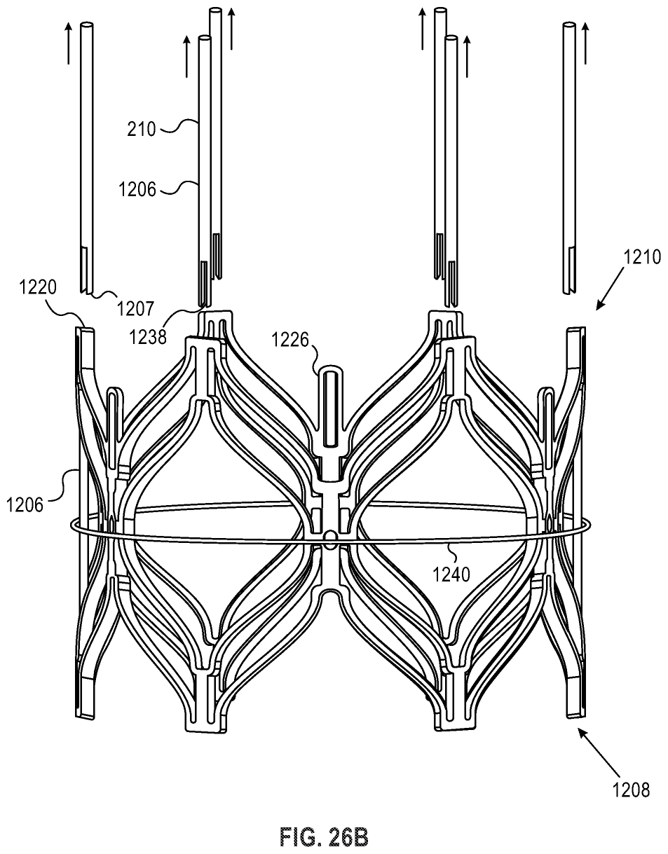

FIGS. 26A-26B illustrate another example of a prosthetic heart valve 1200, which includes a frame 1202 that can be structurally similar to and function in a similar manner as the frame 1102 of prosthetic heart valve 1100, with like number referring to like components. The frame 1202 extends from a first end 1208 to a second end 1210, which can be the inflow end and outflow end, respectively. The frame 1202 includes a plurality of interconnected struts 1212 that define a plurality of cells, such as primary cells 1214 and secondary cells 1216 in the illustrated example. The primary cells 1214 and secondary cells 1216 are interconnected at their ends by vertical struts 1218 that form apices 1220 at the first end 1208 and the second end 1210 of the frame 1202. The prosthetic valve 1200 further includes a valvular structure which can be structurally similar to and function in a similar manner as the valvular structure 104 of prosthetic heart valve 100 (not shown in FIGS. 26A-26B for clarity). The frame 1202 can include a plurality of vertical struts 1228 disposed circumferentially between adjacent pairs of the primary cells 1214. The frame can further include a plurality of leaflet attachment structures, such as commissure windows 1226 in the illustrated example, extending from at least some of the vertical struts 1228.

The prosthetic valve 1200 further actuation members 1206 (e.g., six actuators in the illustrated example) which are coupled to the frame 1202 and are configured to adjust expansion of the frame 1202 from a compressed or a partially radially-expanded state (e.g., FIG. 26A) to a plurality of configurations including one or more functional or expanded configurations (e.g., FIG. 26B). In some implementations, prosthetic valve 1200 can include a fully self-expandable frame 1202. The actuation members 1206 of the prosthetic heart valve 1200 are mounted to and spaced circumferentially around the frame 1202. In the illustrated example, the prosthetic heart valve 1200 comprises six actuation members 1206. In other examples, the prosthetic heart valve can comprise fewer or more than six actuation members (e.g., 1-5 or 7-15).

The actuation members 1206 can be rotatably coupled to the frame 1202, such that they can rotate about their axes while remaining immovable in the circumferential direction with respect to the frame 1202. The actuation members 1206 can be also axially movable relative to the frame 1202. For example, the actuation members 1206 can be formed as relatively rigid rod members axially extending through channels formed in vertical struts 1118 extending from the apices 1220 at the outflow end 1210 of the frame 1202, such that the actuation members 1206 can freely rotate within, as well as slide axially through, these channels. The actuation members 1206 further comprise actuation member slots 1238 extending proximally from free ended terminal ends 1207 of the actuation members 1206. The terminal ends 1207 and actuation member slots 1238 can be positioned within the secondary cells 1216 as shown in the illustrated example.

The prosthetic heart valve 1200 further comprises a flexible tension member 1240 extending through the actuation member slots 1238 and encircling the frame 1202. As mentioned, the prosthetic heart valve 1200 can be configured to radially self-expand from a radially compressed state to a partially or fully expanded state under resilient and/or elastic forces of the prosthetic heart valve frame 1202. The flexible tension member 1240 can be utilized to retain the frame 1202 in a compressed or partially expanded diameter, while increasing the diameter of the flexible tension member 1240 can allow the frame 1202 to expand further, potentially up to a fully expanded or functional diameter. Otherwise stated, the flexible tension member 1240 can apply a radially inwardly directed force to the frame 1202 that can be gradually lessened such that the frame 1202 can expand at a controlled rate (e.g., a rate selected by the physician) to a selected diameter.

Each actuation member 1206 can be coupled to an actuator assembly 209 and/or a delivery system actuator 210 of the delivery apparatus 200 previously discussed. In some implementations, each actuation member 1206 can be affixed to a delivery system actuator 210, or be integrally formed with a delivery system actuator 210, such as being formed as a continuous extension of the delivery system actuator 210 of the delivery apparatus 200. The delivery system actuators 210 can be configured to apply rotational movement to the corresponding actuation members 1206 they are attached to or integrally formed with, in either direction (i.e., clockwise or counterclockwise), to compress or allow self-expansion of the prosthetic heart valve 1200.

The flexible tension member 1240 serves as a restraining mechanism, and can comprise a wire, string, and/or cable. As mentioned, the flexible tension member 1240 extends circumferentially around the frame 1202, and through the actuation member slots 1238. In some examples, the flexible tension member 1240 can be weaved through cells 1214, 1216 of the frame 1202, for example by weaving it in an in-and-out pattern along struts 1212 and/or vertical struts 1228 of the frame 1202. In other examples, the flexible tension member 1240 can be disposed entirely around the frame 1202, except for the portions passing through actuation member slots 1238. In some examples, the frame 1202 can comprise a plurality of additional eyelets or apertures through which portions of the flexible tension member 1240 can be threaded.

The flexible tension member 1240 can extend at least partially around the circumference of the frame 1202. In the illustrated example, the flexible tension member 1240 extends around frame 1202 such that it forms a single loop encircling the frame 1202. In other examples, the flexible tension member 1240 can be disposed such that it extends around the frame 1202 in a continuous helical manner defining a plurality of loops. In still other examples, the flexible tension member 1240 can be disposed such that it spans a distance less than the full circumference of the frame 1202. In some cases, the flexible tension member 1240 can be attached to the frame 1202 at one or more locations, for example by forming knots around struts or posts of the frame 1202 at one or two ends of the flexible tension member 1240.

FIG. 26B shows the frame 1202 in a fully radially expanded state. In order to compress it to a partially radially-expanded state as shown in FIG. 26A, or to a fully radially-compressed or crimped state (similar to the state of frame 102 in FIG. 2C, for example), the actuator assemblies 209 and/or a delivery system actuators 210 of the delivery apparatus 200 can be rotated in a second rotational direction (e.g., counterclockwise in the illustration of FIG. 26A), resulting in a rotational movement of the actuation members 1206 in the same rotational direction. As the actuation members 1206 rotate around their respective central axes, local loops 1242 of the flexible tension member 1240 are formed around the respective actuation members 1206, thereby tensioning the flexible tension member 1240 and shortening the lengths of the adjustable loop sections circumferentially extending between adjacent actuation members 1206, in a manner that will reduce the diameter defined by the flexible tension member 1240 and compress the frame 1202 therewith, for example, from the radially expanded state of FIG. 26B to the partially radially-expanded state as shown in FIG. 26A, wherein continued rotation will further compress the frame 1202 to the radially-compressed or crimped state (similar to the state of frame 102 in FIG. 2C, for example). The actuation member slots 1238 are desirably positioned approximately halfway between the inflow end 1208 and the outflow end 1210 of the prosthetic valve 1200 so that the restraining force of the flexible tension member 1240, when restricting the frame 1202 to a fully or partially compressed diameter, is equally distributed along the length of the frame.

When the prosthetic valve 1200 is deployed from the delivery sheath 218, it can be allowed to self-expand by rotating the actuator assemblies 209 and/or a delivery system actuators 210 of the delivery apparatus 200 in the first rotational direction (e.g. clockwise), resulting in similar rotation of the actuation members 1206 so as to unravel the local loops 1242 and allow the flexible tension member 1240, and the frame 1202 encircled thereby, to increase in diameter, for example up to the radially expanded state shown in FIG. 26B. After full expansion is achieved, the actuator assemblies 209 and/or a delivery system actuators 210 of the delivery apparatus 200 can be proximally pulled away from the prosthetic valve 1200, along with the actuation members 1206 attached thereto or formed as portions thereof, such that actuation member slots 1238 slide over and away from the flexible tension member 1240, allowing retrieval of the delivery apparatus 200 from the patient's body while the prosthetic valve 1200, optionally with the flexible tension member 1240 left around the frame 1202, remains at the site of implantation.

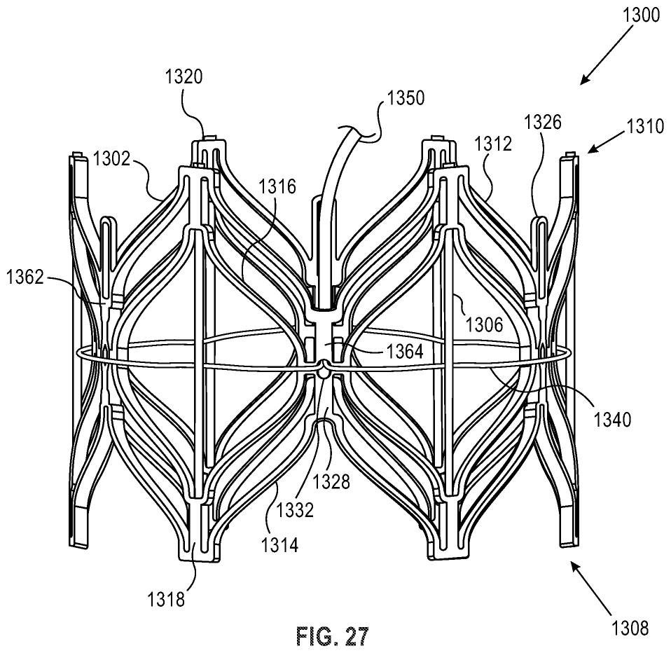

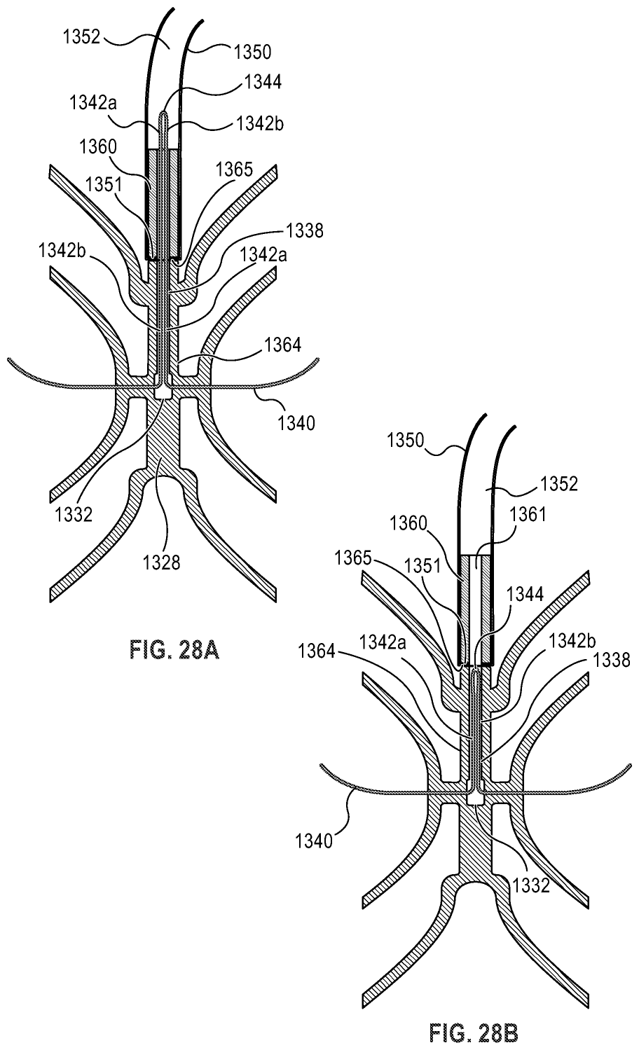

FIG. 27 illustrates another example of a prosthetic heart valve 1300, which includes a frame 1302 that can be structurally similar to and function in a similar manner as the frame 102 of prosthetic heart valve 100, with like number referring to like components. A delivery apparatus used in combination with a prosthetic heart valve 1300 can include a resistance-based expansion control mechanism that includes a resistance feature 1360 shown in FIGS. 28A-28B. The frame 1302 extends from a first end 1308 to a second end 1310, which can be the inflow end and outflow end, respectively. The frame 1302 includes a plurality of interconnected struts 1312 that define a plurality of cells, such as primary cells 1314 and secondary cells 1316 in the illustrated example. The primary cells 1314 and secondary cells 1316 are interconnected at their ends by vertical struts 1318 that form apices 1320 at the first end 1308 and the second end 1310 of the frame 1302. The prosthetic valve 1300 further includes a valvular structure 1304 which can be structurally similar to and function in a similar manner as the valvular structure 104 of prosthetic heart valve 100, including a plurality of leaflets 1334 joined together to form commissures 1336.

The frame 1302 can include a plurality of vertical struts 1328 disposed circumferentially between adjacent pairs of the primary cells 1314. The frame 1302 also comprises at least one aperture 1332 disposed in at least one of the vertical struts 1328. The frame can further include a plurality of leaflet attachment structures, such as commissure windows 1326 in the illustrated example, extending from at least some of the vertical struts 1328. The number of vertical struts 1328 can be greater than the number of commissure windows 1326, such that the vertical struts 1328 can include commissural vertical struts 1362, defined as vertical struts 1328 that include commissure windows 1326 extending therefrom, and non-commissural vertical posts 1364, defined as vertical struts 1328 that do not include commissure windows extending therefrom.

In some implementations, the prosthetic heart valve 1300 can further include actuation members 1306 (e.g., six actuators in the illustrated example) that can be coupled to the frame 1302 and configured to adjust expansion of the frame 1302 from a partially-expanded configuration to a plurality of configurations including one or more functional or expanded configurations. In such implementations, the frame 1302 can be configured as a partially self-expandable frame, configured to function in the same manner described above with respect to actuation members 106 of prosthetic heart valve 100. In alternative implementations, the frame 1302 can be configured as a fully self-expandable frame, in which case, the prosthetic heart valve 1300 need not include the actuator members 1306.

At least one of the vertical struts 1328, and in particular, at least one of the non-commissural vertical posts 1364, further includes a channel 1338 formed therein and extending between aperture 1332 and proximal end 1365 of the non-commissural vertical post 1364. The prosthetic heart valve 1300 further comprises a flexible tension member 1340 encircling the frame 1302. The flexible tension member 1340 can comprise a wire, string, and/or cable. At least a portion of the flexible tension member 1340 extends through the channel 1338, at least in a radially compressed state or a partially radially-expanded state of the frame 1302. The flexible tension member 1340 can apply a radially inwardly directed force to the frame 1302 that can be gradually lessened such that the frame 1302 can expand at a controlled rate (e.g., a rate dictated by a coefficient of friction between the flexible tension member 1340 and a resistance feature, as will be described in greater detail below) to a selected diameter.

A delivery device or apparatus of the prosthetic heart valve 1300 includes at least one delivery system shaft 1350 defining a lumen 1352. The delivery system shaft 1350 further comprises a resistance feature 1360 disposed within its lumen 1352 extending from a distal end 1351 thereof, as in the illustrated example, or positioned within the lumen 1352 proximal to the distal end 1351 of the delivery system shaft 1350 in other examples. In some implementations, the resistance feature 1360 can define an internal passage 1361 in the form of an axially extending central channel formed therethrough and defining a friction surface configured to frictionally engage with the local loop of the flexible tension member 1340. In other implementations, the resistance feature 1360 can define an outer cutout face distanced from an inner surface of the delivery system shaft 1350, such that the internal passage 1361 is formed between the exposed outer surface of the resistance feature 1360, which is the friction surface configured to frictionally engage with the local loop of the flexible tension member 1340, and internal surface of the delivery system shaft 1350.

Resistance feature 1360 can be made of any of the materials previously discussed in connection with resistance feature 506, including biocompatible polymers or elastomers, particularly silicone, flouroelastomer, perflouroelastomer, ethylene propylene, nitrile rubber, or a combination thereof. When the resistance feature 1360 comprises a high friction material, it can be formed within the delivery system shaft 1350 or can be a separate component such as an O-ring or gasket that is set in within the delivery system shaft 1350. Resistance feature 1360 can also include any of the plurality of mechanical features discussed above that would be suitable for resistance feature 1360. Resistance feature 1360 can further incorporate any combination of suitable mechanical features and materials as previously discussed.

FIG. 28A shows a cross-sectional view of a portion of the frame 1302 including the non-commissural vertical post 1364 and a distal portion of a delivery system shaft 1350 of a delivery apparatus that can be releasably coupled thereto, when the prosthetic heart valve 1300 is in a radially compressed state. In this state, a portion of the flexible tension member 1340 forms a local loop 1344 that extends through the channel 1338 and the delivery system shaft 1350, passing along or through the resistance feature 1360, such as along or though the internal passage 1361, such that at least a portion of the local loop 1344 is in frictional contact with the resistance feature 1360. The proximal end of the local loop 1344 can be proximal to the resistance feature 1360, disposed within the lumen 1352 as illustrated in FIG. 28A, or it can be disposed within resistance feature 1360, such as terminating along or in the internal passage 1361.

The local loop 1344 can include a first portion 1342a and a second portion 1342b parallelly extending through the channel 1338 from the aperture 1332 to the proximal end of the local loop 1344. The first and second portion 1342a, 1342b of the flexible tension member 1340 extend from the local loop 1344 through the aperture 1332, from which the remainder of the flexible tension member 1340 can extend around the frame 1302. In some examples, the portion of the flexible tension member 1340 extending out of the aperture 1332 can be weaved through cells 1314, 1316 of the frame 1302, for example by weaving it in an in-and-out pattern along struts 1312 and/or vertical struts 1328 of the frame 1302. In other examples, the portion of the flexible tension member 1340 extending out of the aperture 1332 can be disposed entirely around the frame 1302. In some examples, the frame 1302 can comprise a plurality of additional eyelets or apertures through which portions of the flexible tension member 1340 portion that extends out of the aperture 1332 can be threaded.

The portion of the flexible tension member 1340 extending out of the aperture 1332 can extend at least partially around the circumference of the frame 1302. In the illustrated example, the portion of the flexible tension member 1340 extending out of the aperture 1332 extends around frame 1302 such that it forms a single loop encircling the frame 1302. In other examples, the portion of the flexible tension member 1340 extending out of the aperture 1332 can be disposed such that it extends around the frame 1302 in a continuous helical manner defining a plurality of loops. In still other examples, the portion of the flexible tension member 1340 extending out of the aperture 1332 can be disposed such that it spans a distance less than the full circumference of the frame 1302. In some cases, the portion of the flexible tension member 1340 extending out of the aperture 1332 can be attached to the frame 1302 at one or more locations, for example by forming knots around struts or posts of the frame 1302 at one or two ends of the flexible tension member 1340.

A first coefficient of friction exists between the flexible tension member 1340 and the interior surface of the channel 1338, and a second coefficient of friction exists between the flexible tension member 1340 and the resistance feature 1360, wherein the second coefficient of friction is higher than the first coefficient of friction. In this way, the slidable contact between the high friction material of the resistance feature 1360 can cause a greater friction force to resist the axial motion of the local loop 1344 of flexible tension member 1340 than would occur without the resistance feature. During the self-expansion of the prosthetic heart valve 1300, this elevated friction force can thus partially oppose, but not wholly prevent, the expansion of the prosthetic heart valve 1300, reducing the rate of prosthetic heart valve self-expansion and controlling any jumping of the prosthetic heart valve 1300 at the implantation site during the self-expansion stage of the implantation procedure.

When the local loop 1344 moves relative to the delivery system shaft 1350 through or along the resistance feature 1360, a friction force will be generated based on the coefficient of dynamic friction between the flexible tension member 1340 and the material of the resistance feature 1360. The coefficient of dynamic friction between the channel 1338 and the flexible tension member 1340 can be 0.6 or less, such as 0.5 or less, 0.4 or less, or 0.3 or less, than the dynamic friction between the flexible tension member 1340 and the material of the resistance feature 1360.

The internal passage 1361 has an effective diameter, which can be the diameter of the internal passage if provided as a tubular passage exhibiting a circular cross-section, or can be defined as the shortest distance between opposing edges of the internal passage when defined to have a non-circular cross-section. The flexible tension member 1340 has a thickness, defined as the free thickness it exhibits in a free or uncompressed state thereof. When the flexible tension member 1340 comprises a local loop 1344 with two portions 1342a and 1342b extending through the internal passage 1361, the effective diameter of the internal passage 1361 can be equal to or slightly narrower than twice the thickness of the flexible tension member 1340, in which case both portions 1342a, 1342b can be tightly pressed against or somewhat squeezed within the internal passage 1361, forcing the local loop 1344 to contact and frictionally engage the surrounding wall(s) of the internal passage 1361.

In an alternative example, the resistance feature 1360 can instead comprise a plurality of mechanical features configured to provide resistance to the axial motion of the local loop 1344 passing therealong or therethrough. The plurality of mechanical features can include a set of features configured to slidably engage with the local loop 1344. The mechanical features may be configured to partially resist the axial motion of the local loop 1344 through the channel 1338 while the prosthetic heart valve 1300 is self-expanding.

As depicted in FIG. 28A, the delivery system shaft 1350 of the delivery apparatus remains in contact with the non-commissural vertical post 1364 to define a continuous path between the channel 1338 and the internal passage formed by the resistance feature 1360, such that the local loop 1344 remains in slidable contact with the resistance feature 1360 while the valve is self-expanding. This allows the resistance feature 1360 to impart a force on the local loop 1344 that partially resists, but does not fully stop, its axial motion along and/or through the channel 1338.

When the axial motion of the local loop 1344 is resisted, the rate of expansion of the prosthetic heart valve 1300 is reduced from a natural expansion rate to a controlled expansion rate that is lower than the natural expansion rate. In this way, any tendency of the prosthetic heart valve 1300 to jump or become misaligned during self-expansion can be avoided. In certain examples, the resistance feature 1360 is sized and configured such that it is in contact with the local loop 1344 from the radially compressed delivery state (which can be similar to the state shown for prosthetic heart valve 100 in FIG. 2C) to the partially expanded state, or even, if the frame 1302 is fully radially self-expandable, to a functional or fully radially expanded state (such as shown in FIG. 27). In this manner, the contact between the resistance feature 1360 and the local loop 1344 can reduce the rate of expansion of the prosthetic valve 1300 throughout the entire self-expansion range of the prosthetic valve 1300.

In certain examples, the frame 1302 is partially self-expandable, and the upper limit of the self-expansion range (the diameter of the prosthetic valve 1300 when it stops expanding under the resiliency of the frame) is selected to be less than the diameter of the annulus of the native heart valve in which the prosthetic valve 1300 is to be implanted. Thus, in such examples, when prosthetic valve 1300 reaches the upper limit of the self-expansion range, the prosthetic valve 1300 does not contact the surrounding native annulus.

FIG. 28A can represent, in such examples, the location of the local loop 1344 relative to the channel 1338 and the delivery system shaft 1350 at the upper limit of the self-expansion range. As shown, the resistance-based expansion control mechanism can be configured such that, when the prosthetic heart valve 1300 reaches the upper limit of the self-expansion range, the flexible tension member 1340 comes fully out of engagement with the resistance feature 1360. In this way, the resistance feature 1360 can be prevented from providing any resistance to enlargement in diameter of the flexible tension member 1340 during subsequent expansion from the partially expanded state to the fully expanded state. As the prosthetic valve 1300 self-expands, the frame 1302 can apply tension forces that will expand the flexible tension member 1340 therewith. After the prosthetic heart valve 1300 has self-expanded to the partially radially expanded condition, delivery system actuator (such as delivery system actuators 210) can be actuated by the user to actuate the actuation members 1306 to further radially expand the prosthetic valve 1300 along with the flexible tension member 1340 surrounding it.

In other examples, as mentioned above, the frame 1302 is fully self-expandable, such that when prosthetic valve 1300 reaches the upper limit of the self-expansion range, the prosthetic valve 1300 contacts the surrounding native annulus. FIG. 28A can represent, in such examples, the location of the local loop 1344 relative to the channel 1338 and the delivery system shaft 1350 when the prosthetic valve 1300 reaches its fully expanded or functional diameter. As shown, the resistance-based expansion control mechanism can be configured such that, when the prosthetic heart valve 1300 reaches the fully expanded or functional diameter, the flexible tension member 1340 comes fully out of engagement with the resistance feature 1360.

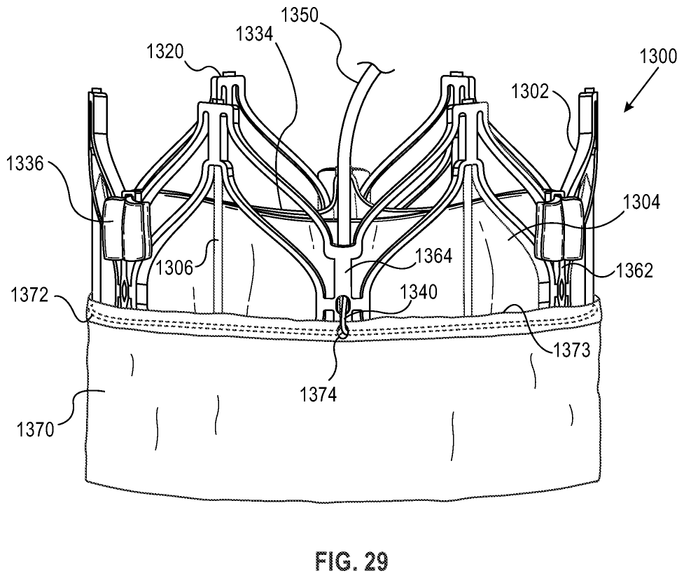

As mentioned with respect to prosthetic valve 100, the prosthetic valve 1300 can include one or more skirts, such as an inner skirt or an outer skirt. One example of an outer skirt 1370 mounted on the outer surface of the frame 1302, optionally configured to function as sealing member for prosthetic valve 1300, is shown in FIG. 29. In some examples, the outer skirt 1370 can include a sleeve 1372 that is configured to receive the portion of the flexible tension member 1340 extending out of the aperture 1332. As one example, the sleeve 1372 can form a pocket in the outer skirt 1370 that extends circumferentially around the frame 1302. The sleeve 1372 can be included at an outflow edge portion 1373 of the outer skirt 1370. The sleeve 1372 can be formed by folding the outflow edge portion 1373 back against the main body of the skirt and then connecting (e.g., stitching) the folded flap against the main body of the skirt.

As shown in the illustrated example, the sleeve 1372 desirably can be positioned approximately halfway between the inflow end 1308 and the outflow end 1310 of the prosthetic heart valve 1300 so that the restraining force of the flexible tension member 1340, when engaged with the resistance feature 1360, is equally distributed along the length of the frame. In alternative embodiments, the sleeve 1372 can be closer to the inflow end 1308 or closer to the outflow end 1310 of the prosthetic valve 1300, which can result in the prosthetic heart valve 1300 assuming a substantially tapered V-shaped or A-shaped configuration (having one end thereof narrower than the opposite end) upon expansion. The flexible tension member 1340 can extend through the sleeve 1372 and circumferentially around the frame 1302. The flexible tension member 1340 can exit the channel 1338 via aperture 1332 and enter the sleeve 1372 through an opening 1374 in the skirt.

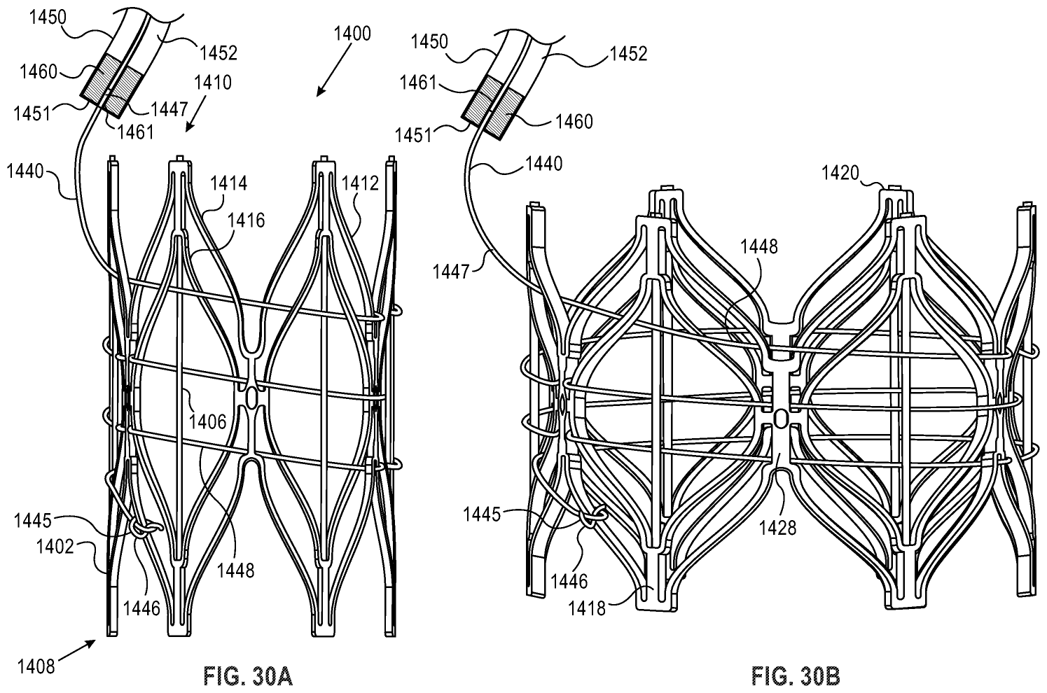

FIGS. 30A-30B illustrate another example of a prosthetic heart valve 1400, which includes a frame 1402 that can be structurally similar to and function in a similar manner as the frame 102 of prosthetic heart valve 100, with like number referring to like components. A delivery apparatus used in combination with a prosthetic heart valve 1400 can include a resistance-based expansion control mechanism that includes a resistance feature 1460 shown in FIGS. 30A-30B. The frame 1402 extends from a first end 1408 to a second end 1410, which can be the inflow end and outflow end, respectively. The frame 1402 includes a plurality of interconnected struts 1412 that define a plurality of cells, such as primary cells 1414 and secondary cells 1416 in the illustrated example. The primary cells 1414 and secondary cells 1416 are interconnected at their ends by vertical struts 1418 that form apices 1420 at the first end 1408 and the second end 1410 of the frame 1402.

The prosthetic valve 1400 further includes a valvular structure which can be structurally similar to and function in a similar manner as the valvular structure 104 of prosthetic heart valve 100 (not shown in FIG. 27 for clarity). The frame 1402 can include a plurality of vertical struts 1428 disposed circumferentially between adjacent pairs of the primary cells 1414. The frame can further include a plurality of leaflet attachment structures, such as commissure windows 1426 in the illustrated example, extending from at least some of the vertical struts 1428.

In some implementations, the prosthetic heart valve 1400 can further include actuation members 1406 (e.g., six actuators in the illustrated example) that can be coupled to the frame 1402 and configured to adjust expansion of the frame 1402 from a partially-expanded configuration to a plurality of configurations including one or more functional or expanded configurations. In such implementations, the frame 1402 can be configured as a partially self-expandable frame, configured to function in the same manner described above with respect to actuation members 106 of prosthetic heart valve 100. In alternative implementations, the frame 1402 can be configured as a fully self-expandable frame, in which case, the prosthetic heart valve 1400 need not include the actuator members 1406.

The prosthetic heart valve 1400 further comprises a flexible tension member 1440 encircling the frame 1402. The flexible tension member 1440 can comprise a wire, string, and/or cable, and can be coupled to the frame 1402 by, for example, being weaved through cells 1414, 1416 of the frame 1402, including optionally being weaved in an in-and-out pattern along struts 1412 and/or vertical struts 1428 of the frame 1402. In other examples, the frame 1402 can comprise a plurality of eyelets or apertures through which portions of the flexible tension member 1440 can be threaded. In yet further examples, the flexible tension member 1440 in indirectly coupled to the frame 1402, such as by being coupled to a skirt which is in turn attached to the frame. For example, the flexible tension member 1440 can extend through a sleeve of a skirt, that can be similar to sleeve 1372 of outer skirt 1370 described above with respect to prosthetic heart valve 1300. Such a sleeve can be circumferentially disposed to form a single loop around the frame, similar to sleeve 1372 illustrated in FIG. 29, or can be a helically formed sleeve through which a flexible tension member 1440 can extend following a pattern similar to that illustrated in FIGS. 30A-30B. In the illustrated example, the flexible tension member 1440 is shown to be woven through the cells 1414, 1416 of the frame 1402 in an in-and-out pattern such that some portions of the flexible tension member 1440 are disposed on a radially outer surface of the frame 1402 and other portions of the flexible tension member 1440 are disposed on a radially inner surface of the frame 1402. The flexible tension member 1440 can apply a radially inwardly directed force to the frame 1402 that can be gradually lessened such that the frame 1402 can expand at a controlled rate (e.g., a rate dictated by a coefficient of friction between the flexible tension member 1440 and a resistance feature, as will be described in greater detail below) to a selected diameter.

A delivery device or apparatus of the prosthetic heart valve 1400 includes at least one delivery system shaft 1450 defining a lumen 1452. The delivery system shaft 1450 further comprises a resistance feature 1460 disposed within its lumen 1452 extending from a distal end 1451 thereof, as in the illustrated example, or positioned within the lumen 1452 proximal to the distal end 1451 of the delivery system shaft 1450 in other examples. In some implementations, the resistance feature 1460 can define an internal passage 1461 in the form of an axially central channel formed therethrough and defining a friction surface configured to frictionally engage with the second end portion 1447 of the flexible tension member 1440. In other implementations, the resistance feature 1460 can define an outer cutout face distanced from an inner surface of the delivery system shaft 1450, such that the internal passage 1461 is formed between the exposed outer surface of the resistance feature 1460, which is the friction surface configured to frictionally engage with the second end portion 1447 of the flexible tension member 1440, and internal surface of the delivery system shaft 1450.

Resistance feature 1460 can be made of any of the materials previously discussed in connection with resistance feature 506, including biocompatible polymers or elastomers, particularly silicone, flouroelastomer, perflouroelastomer, ethylene propylene, nitrile rubber, or a combination thereof. When the resistance feature 1460 comprises a high friction material, it can be formed within the delivery system shaft 1450 or can be a separate component such as an O-ring or gasket that is set in within the delivery system shaft 1450. Resistance feature 1460 can also include any of the plurality of mechanical features discussed above that would be suitable for resistance feature 1460. Resistance feature 1460 can further incorporate any combination of suitable mechanical features and materials as previously discussed.

The flexible tension member 1440 can be coupled to the frame 1402 at a first end portion 1445, such as being releasably coupled to the frame 1402 by a knot 1446, and can have a second end portion 1447, opposite to the first end portion 1445, extending from the frame 1402 into the delivery system shaft 1450, being in frictional engagement with the resistance feature 1460 at least in a radially compressed state or a partially radially-expanded state of the frame 1402. In alternative implementations, when the flexible tension member 1440 extends around an outer skirt, or through a sleeve formed in an outer skirt, a first end portion 1445 can be similarly attached to the skirt, such as being sutured or tied thereto.

The flexible tension member 1440 can extend at least partially around the circumference of the frame 1402. For example, the flexible tension member 1440 can be disposed such that it extends around the frame 1402 in a continuous helical manner defining a plurality of loops 1448. In the illustrated example, the flexible tension member 1440 extends helically around frame 1402 such that it defines three loops 1448. However, in other examples, the flexible tension member 1440 can define a greater or fewer number of loops, for example, a single loop. In still other examples, the flexible tension member 1440 can be disposed such that it spans a distance less than the full circumference of the frame 1402.

In the illustrated example, the flexible tension member 1440 is shown to be attached at its first end 1445 to the frame by a knot 1446. In other examples, the flexible tension member 1440 can be coupled to the frame using one or more clips, hooks, by being glued to the frame 1402, or by any other releasable or non-releasable attachment mechanisms.

FIG. 30A can represent the frame 1302 in either a partially radially expanded state or in a radially compressed state. In a radially compressed state, second end portion 1447 extends along or through the resistance feature 1460, such as along or though the internal passage 1461, such that at least part of the second end portion 1447 is in frictional contact with the resistance feature 1460. The second end portion 1447 can extend further into lumen 1452 proximally to resistance feature 1460 as illustrated in FIGS. 30A-30B, or it can be disposed within resistance feature 1460, such as terminating along or in the internal passage 1461.

The flexible tension member 1440, when engaged with resistance feature 1460, can be used to gradually allow the expansion of prosthetic heart valve 1400. A first coefficient of friction exists between the flexible tension member 1440 and the inner or outer surface of the frame 1402, and a second coefficient of friction exists between the flexible tension member 1440 and the resistance feature 1460, wherein the second coefficient of friction is higher than the first coefficient of friction. In this way, the slidable contact between the high friction material of the resistance feature 1460 can cause a greater friction force to resist free spontaneous diameter enlargement of loops 1448 of the flexible tension member 1440 that could take place in the absence of the resistance feature. During the self-expansion of the prosthetic heart valve 1400, this elevated friction force can thus partially oppose, but not wholly prevent, the expansion of the prosthetic heart valve 1400, reducing the rate of prosthetic heart valve self-expansion and controlling any jumping of the prosthetic heart valve 1400 at the implantation site during the self-expansion stage of the implantation procedure.

When the second end portion 1447 moves relative to the delivery system shaft 1450 through or along the resistance feature 1460, a friction force will be generated based on the coefficient of dynamic friction between the flexible tension member 1440 and the material of the resistance feature 1460. The coefficient of dynamic friction between any surface of the frame 1402 and the flexible tension member 1440 can be 0.6 or less, such as 0.5 or less, 0.4 or less, or 0.3 or less, than the dynamic friction between the flexible tension member 1440 and the material of the resistance feature 1460.

The internal passage 1461 has an effective diameter, which can be the diameter of the internal passage if provided as a tubular passage exhibiting a circular cross-section, or can be defined as the shortest distance between opposing edges of the internal passage when defined to have a non-circular cross-section. The flexible tension member 1440 has a thickness, defined as the free thickness it exhibits in a free or uncompressed state thereof. The effective diameter of the internal passage 1461 can be equal to or slightly narrower than the thickness of the flexible tension member 1440, so as to tightly press it against or squeeze it within the internal passage 1461, forcing the tension member 1440 to contact and frictionally engage the surrounding wall(s) of the internal passage 1461.

In an alternative example, the resistance feature 1460 can instead comprise a plurality of mechanical features configured to provide resistance to the axial motion of the second end portion 1447 passing therealong or therethrough. The plurality of mechanical features can include a set of features configured to slidably engage with the second end portion 1447. The mechanical features may be configured to partially resist the axial motion of the second end portion 1447 relative to the delivery system shaft 1450 while the prosthetic heart valve 1400 is self-expanding.

As depicted in FIG. 30A, the second end portion 1447 remains in slidable contact with the resistance feature 1460 while the valve is self-expanding. This allows the resistance feature 1460 to impart a force on the second end portion 1447 that partially resists, but does not fully stop, its axial motion along and/or through the delivery system shaft 1450. When the axial motion of the second end portion 1447 through delivery system shaft 1450 is resisted, the rate of expansion of the prosthetic heart valve 1400 is reduced from a natural expansion rate to a controlled expansion rate that is lower than the natural expansion rate. In this way, any tendency of the prosthetic heart valve 1400 to jump or become misaligned during self-expansion can be avoided.

In certain examples, the resistance feature 1460 is sized and configured such that it is in contact with the second end portion 1447 from the radially compressed delivery state to the partially expanded state, or even, if the frame 1402 is fully radially self-expandable, to a functional or fully radially expanded state (such as shown in FIG. 30B). In this manner, the contact between the resistance feature 1460 and the second end portion 1447 can reduce the rate of expansion of the prosthetic valve 1400 throughout the entire self-expansion range of the prosthetic valve 1400.

In certain examples, the frame 1402 is partially self-expandable, and the upper limit of the self-expansion range (the diameter of the prosthetic valve 1400 when it stops expanding under the resiliency of the frame) is selected to be less than the diameter of the annulus of the native heart valve in which the prosthetic valve 1400 is to be implanted. Thus, in such examples, when prosthetic valve 1400 reaches the upper limit of the self-expansion range, the prosthetic valve 1400 does not contact the surrounding native annulus.

FIG. 30A can represent, in such examples, the configuration of the frame 1402 and loops 1448 of the flexible tension member 1440 at the upper limit of the self-expansion range. While flexible tension member 1440 is illustrated to remain partially within the delivery system shaft 1450 and in engagement with the resistance feature 1460, the resistance-based expansion control mechanism can be configured such that, when the prosthetic heart valve 1400 reaches the upper limit of the self-expansion range, the flexible tension member 1440 comes fully out of engagement with the resistance feature 1460 (not shown). In this way, the resistance feature 1460 can be prevented from providing any resistance to enlargement in diameter of the flexible tension member 1440 during subsequent expansion from the partially expanded state to the fully expanded state. As the prosthetic valve 1400 self-expands, the frame 1402 can apply tension forces that will expand the flexible tension member 1440 therewith. After the prosthetic heart valve 1400 has self-expanded to the partially radially expanded condition, delivery system actuator (such as delivery system actuators 210) can be actuated by the user to actuate the actuation members 1406 to further radially expand the prosthetic valve 1400 along with the flexible tension member 1440 surrounding it.

Alternatively, the flexible tension member 1440 can remain in engagement with the resistance feature 1460 even after partial expansion of the frame 1402 as in the illustrated example, in which case, the actuation members 1406 can apply forces that overcome the frictional resistance and expand the frame to its fully radially expanded or functional diameter, as shown in FIG. 30B.

In other examples, as mentioned above, the frame 1402 is fully self-expandable, such that when prosthetic valve 1400 reaches the upper limit of the self-expansion range, the prosthetic valve 1400 contacts the surrounding native annulus. FIG. 30B can represent, in such examples, the configuration of the frame 1402 and loops 1448 of the flexible tension member 1440 when the prosthetic valve 1300 reaches its fully expanded or functional diameter. While flexible tension member 1440 is illustrated to remain partially within the delivery system shaft 1450 and in engagement with the resistance feature 1460, the resistance-based expansion control mechanism can be configured such that, when the prosthetic heart valve 1400 reaches the fully expanded or functional diameter, the flexible tension member 1440 comes fully out of engagement with the resistance feature 1460 (not shown). Alternatively, the flexible tension member 1440 can remain in engagement with the resistance feature 1460 during the entire range up to full radial expansion, as shown in FIG. 30B, providing controlled expansion of the frame 1402 throughout the entire range.

In some implementations, once the prosthetic heart valve 1400 has been fully expanded, the tension in the flexible tension member 1440 can be fully released and the flexible tension member 1440 can be uncoupled from the delivery system shaft 1450, or it can be uncoupled from the frame 1402 and retrieved along with the delivery system shaft 1450 from the patient's body.

The term “medical assembly”, as used throughout the specification and the claims, refers to an assembly that includes a prosthetic heart valve (including any prosthetic heart valve disclosed herein) and a delivery device (such as delivery apparatus 200) that can include a delivery system actuator (e.g., delivery system actuator 210) and/or a delivery system shaft (e.g., delivery system shaft 1350 or 1450).

It is to be understood that any other combination of mechanical features which impart a partial resistance to the axial motion of the delivery system actuator within the channel that resists, but does not completely stop the axial motion of the delivery system actuator and the radial expansion of the prosthetic heart valve may be used. It is also to be appreciated that a resistance features comprising a plurality of mechanical features may additionally include any of the high friction materials discussed above.

In all such examples, the position of the resistance features may be selected such that the resistance features are engaged when the prosthetic heart valve is self-expanding and to come out of engagement when the prosthetic heart valve has finished self-expanding. In the case of a partially mechanically expandable prosthetic heart valve, the resistance features may be engaged while the prosthetic heart valve is self-expanding from the radially compressed state to the partially radially expanded state, but disengaged when the prosthetic heart valve is being mechanically expanded from the partially radially expanded state to the fully radially expanded state. In the case of a fully self-expanding prosthetic heart valve, the resistance feature can remain engaged for the entire expansion from the radially compressed state to the fully radially expanded system, coming out of engagement when the prosthetic heart valve is in its final, fully expanded configuration.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A medical assembly comprising a prosthetic heart valve having a radially expandable annular frame. The frame comprises at least one frame portion defining an axially extending channel, a valvular structure disposed within the frame and configured to regulate the flow of blood through the frame in one direction, and at least one actuation member coupled to the frame and configured to apply an axially directed force to the frame. The prosthetic heart valve is configured to self-expand from a radially compressed state to at least a partially radially expanded state. The medical assembly also comprises a delivery device. The delivery device comprises at least one delivery system actuator releasably coupled to the actuation member and extending through the axially extending channel of the frame. One of an exterior surface of the delivery system actuator and an inner surface the axially extending channel comprises a friction surface and the other of the exterior surface of the delivery system actuator and the inner surface of the axially extending channel comprises an opposing surface that can engage the friction surface, wherein the exterior surface of the delivery system actuator and the inner surface of the axially extending channel have a first coefficient of friction when the friction surface and the opposing surface engage each other and a second coefficient of friction when the friction surface and the opposing surface do not engage each other, wherein the first coefficient of friction is greater than the second coefficient of friction. When the prosthetic heart valve self-expands from the radially compressed state to the partially radially expanded state, the delivery system actuator can slide relative to the axially extending channel and the friction surface slides against the opposing surface to control a rate of expansion of the prosthetic heart valve. The delivery system actuator is configured to move the actuation member in an axial direction to further expand the prosthetic heart valve from the partially radially expanded state to a further radially expanded state.

Example 2. The medical assembly of any example herein, particularly example 1, wherein the delivery device comprises a sheath configured to retain the prosthetic heart valve in the radially compressed state and to deploy the prosthetic heart valve from the sheath to allow the prosthetic heart valve to self-expand.

Example 3. The medical assembly of any example herein, particularly example 1, wherein the delivery device comprises an adjustable loop circumferentially disposed around an exterior of the prosthetic heart valve to retain the prosthetic heart valve in the radially compressed state and to allow the prosthetic heart valve to self-expand when slack is introduced in the adjustable loop.

Example 4. The medical assembly of any example herein, particularly examples 1-3, wherein the delivery system actuator is configured to exert a proximally directed force on the actuation member to radially expand the prosthetic heart valve from the partially radially expanded state to the further radially-expanded state.

Example 5. The medical assembly of any example herein, particularly of examples 1-4, wherein the friction surface is configured to disengage from the opposing surface when the prosthetic heart valve reaches the partially radially expanded state.

Example 6. The medical assembly of any example herein, particularly of examples 1-4, wherein the frame comprises a proximal end portion and a distal end portion, wherein the proximal end portion comprises a plurality of frame apices, wherein the axially extending channel extends through one of the frame apices.

Example 7. The medical assembly of any example herein, particularly of example 6, wherein the actuation member is configured to pass through the axially extending channel when the prosthetic heart valve is fully expanded.

Example 8. The medical assembly of any example herein, particularly of examples 1-7, wherein the frame comprises at least one locking member configured to engage the actuation member and prevent radial compression of the prosthetic heart valve after the prosthetic heart valve is expanded to the further radially expanded state.

Example 9. The medical assembly of any example herein, particularly of examples 1-8, wherein the frame is made of Nitinol.

Example 10. The medical assembly of any example herein, particularly of examples 1-9, wherein the friction surface is on the delivery system actuator and the opposing surface is on the inner surface of the axially extending channel.

Example 11. The medical assembly of any example herein, particularly of examples 1-9, wherein the friction surface is on the inner surface of the axially extending channel and the opposing surface is on the delivery system actuator.

Example 12. The medical assembly of any example herein, particularly of examples 1-9, wherein the opposing surface comprises another friction surface.

Example 13. The medical assembly of any example herein, particularly of examples 1-12, wherein the friction surface comprises a polymer.

Example 14. The medical assembly of any example herein, particularly of claim 13, wherein the polymer is an elastomer.

Example 15. The medical assembly of any example herein, particularly of claim 14, wherein the elastomer is silicone, flouroelastomer, perflouroelastomer, ethylene propylene, nitrile rubber, or a combination thereof.

Example 16. The medical assembly of any example herein, particularly of examples 1-15 wherein the friction surface is configured to disengage completely from the frame and to provide no resistance to axial motion of the delivery system actuator when the prosthetic heart valve has self-expanded to the partially radially expanded state.

Example 17. The medical assembly of any example herein, particularly of examples 1-16, wherein the at least one actuation member comprises a plurality of actuation members and the at least one delivery system actuator comprises a plurality of delivery system actuators releasably coupled to respective actuation members and extending through respective channels of the frame.

Example 18. The medical assembly of any example herein, particularly of claim 17, wherein each delivery system actuator and corresponding channel has a friction surface and an opposing surface.

Example 19. The medical assembly of any example herein, particularly of examples 17-18, wherein the delivery device comprises a plurality of support tubes that abut the frame, wherein each delivery system actuator extends through one of the support tubes.

Example 20. The medical assembly of any example herein, particularly of examples 1-19, wherein the delivery system actuator is configured to be de-coupled from the actuation member after the prosthetic heart valve is expanded to the further radially expanded state so that the delivery device can be removed from a patient's body.

Example 21. A medical assembly, comprising a prosthetic heart valve. The prosthetic heart valve comprises a radially expandable annular frame, a valvular structure disposed within the frame and configured to regulate the flow of blood through the frame in one direction, and at least one actuation member coupled to the frame and configured to apply an axially force to the frame. The frame is configured to self-expand from a radially compressed state to at least a partially radially expanded state. The medical assembly also comprises a delivery device. The delivery device comprises at least one delivery system actuator releasably coupled to the actuation member and at least one support tube disposed around the delivery system actuator and configured to allow the delivery system actuator to extend and move axially therethrough. One of an exterior surface of the delivery system actuator and an inner surface of the support tube comprises a resistance surface and the other of the exterior surface of the delivery system actuator and the inner surface of the support tube comprises an opposing surface that can engage the resistance surface, wherein a coefficient of friction between the delivery system actuator and the support tube is increased when the resistance surface and the opposing surface engage each other. When the frame self-expands from the radially compressed state to the partially radially expanded state, the delivery system actuator slides relative to the support tube and the resistance surface slides against the opposing surface to control a rate of expansion of the frame. The delivery system actuator is configured to move the actuation member in an axial direction to further expand the frame from the partially radially expanded state to a further radially expanded state.

Example 22. The medical assembly of any example herein, particularly of example 21, wherein the delivery device comprises a sheath configured to retain the frame in the radially compressed state and to deploy the frame from the sheath to allow the frame to self-expand.

Example 23. The medical assembly of any example herein, particularly of example 21, wherein the delivery device comprises an adjustable loop circumferentially disposed around an exterior of the frame to retain the frame in the radially compressed state and to allow the frame to self-expand when slack is introduced to the adjustable loop.

Example 24. The medical assembly of any example herein, particularly of examples 21-23, wherein the delivery system actuator is configured to exert an axial force on the actuation member to radially expand the frame from the partially radially expanded state to a further radially-expanded state.

Example 25. The medical assembly of any example herein, particularly of examples 21-24, wherein the resistance surface is configured to disengage from the opposing surface when the frame reaches the partially radially expanded state.

Example 26. The medical assembly of any example herein, particularly of examples 21-25, wherein the frame comprises a proximal end portion and a distal end portion, wherein the proximal end portion comprises a plurality of frame apices.

Example 27. The medical assembly of any example herein, particularly of example 26, wherein the frame comprises at least one channel extending through one of the plurality of frame apices.

Example 28. The medical assembly of any example herein, particularly of example 27, wherein the delivery system actuator extends through the channel.

Example 29. The medical assembly of any example herein, particularly of examples 21-28 wherein the frame comprises at least one locking member configured to engage the actuation member and prevent radial compression of the frame after the frame is expanded to the further radially expanded state.

Example 30. The medical assembly of any example herein, particularly of examples 21-29, wherein the resistance surface is on the delivery system actuator and the opposing surface is on the inner surface of the support tube.

Example 31. The medical assembly of any example herein, particularly of examples 20-29, wherein the resistance surface is on the inner surface of the support tube and the opposing surface is on the delivery system actuator.

Example 32. The medical assembly of any example herein, particularly of examples 20-31, wherein the opposing surface comprises another resistance surface.

Example 33. The medical assembly of any example herein, particularly of examples 21-32, wherein the friction surface comprises a polymer.

Example 34. The medical assembly of any example herein, particularly of example 33, wherein the polymer is an elastomer.

Example 35. The medical assembly of any example herein, particularly of example 34, wherein the elastomer is silicone, flouroelastomer, perflouroelastomer, ethylene propylene, nitrile rubber, or a combination thereof.

Example 36. The medical assembly of any example herein, particularly of examples 21-35, wherein the at least one actuation member comprises a plurality of actuation members and the at least one delivery system actuator comprises a plurality of delivery system actuators releasably coupled to respective actuation members and extending through respective channels of the frame.

Example 37. The medical assembly of any example herein, particularly of example 36, wherein each delivery system actuator and corresponding support tube has a friction surface and an opposing surface.

Example 38. The medical assembly of any example herein, particularly of examples 21-37, wherein the delivery system actuator is configured to be decoupled from the actuation member after the prosthetic heart valve is expanded to the further radially expanded state so that the delivery device can be removed from a patient's body.

Example 39. A prosthetic heart valve, comprising a radially expandable frame. The frame comprises a frame portion defining at least one axially extending channel, an axially extending frame member that extends through the axially extending channel, and a plurality of leaflets disposed within the frame and configured to regulate the flow of blood through the frame in one direction. One of an exterior surface of the axially extending frame member and an inner surface the axially extending channel comprises a friction surface and the other of the exterior surface of the axially extending frame member and the inner surface of the axially extending channel comprises an opposing surface that can engage the friction surface, wherein a friction force between the axially extending frame member and the axially extending channel is increased when the friction surface is engaged with the opposing surface. The prosthetic heart valve is configured to self-expand from a radially compressed state to at least a partially radially expanded state. Example 40. The medical assembly of any example herein, particularly of example 39, wherein the prosthetic heart valve is configured to self-expand from the partially radially expanded state to a fully radially expanded state.

Example 41. The medical assembly of any example herein, particularly of examples 39-40, wherein the friction surface is configured to disengage from the opposing surface when the prosthetic heart valve reaches the partially radially expanded state.

Example 42. The medical assembly of any example herein, particularly of examples 39-40, wherein the friction surface is configured to disengage from the opposing surface when the prosthetic heart valve reaches the fully radially expanded state.

Example 43. The medical assembly of any example herein, particularly of examples 39-42, wherein the frame comprises a proximal end portion and a distal end portion, wherein the proximal end portion comprises a plurality of frame apices wherein the axially extending channel extends through one of the frame apices.

Example 44. The medical assembly of any example herein, particularly of examples 39-43, wherein the frame comprises at least one locking member configured to engage the axially extending frame member and prevent radial compression of the prosthetic heart valve after the prosthetic heart valve is expanded to a radially expanded state.

Example 45. The medical assembly of any example herein, particularly of examples 39-44, wherein the frame is made of nitinol.

Example 46. The medical assembly of any example herein, particularly of examples 39-45, wherein the friction surface is on the axially extending frame member and the opposing surface is on the inner surface of the axially extending channel.

Example 47. The medical assembly of any example herein, particularly of examples 39-45, wherein the friction surface is on the inner surface of the axially extending channel and the opposing surface is on the axially extending frame member.

Example 48. The medical assembly of any example herein, particularly of examples 39-45, wherein the opposing surface comprises another friction surface.

Example 49. The medical assembly of any example herein, particularly of examples 39-48, wherein the axially extending frame member comprises a plurality of axially extending frame members, and the axially extending channel comprises a plurality of channels.

Example 50. The medical assembly of any example herein, particularly of example 49, wherein each axially extending frame member and each corresponding channel has a friction surface and an opposing surface.

Example 51. The medical assembly of any example herein, particularly of examples 39-50, wherein the friction surface comprises a polymer.

Example 52. The medical assembly of any example herein, particularly of example 51, wherein the polymer is an elastomer.

Example 53. The medical assembly of any example herein, particularly of example 52, wherein the elastomer is silicone, flouroelastomer, perflouroelastomer, ethylene propylene, nitrile rubber, or a combination thereof.

Example 54. A medical assembly comprising a prosthetic heart valve. The prosthetic heart valve comprises a radially expandable annular frame. The frame comprises a plurality of frame portions defining a plurality of axially extending channels. The prosthetic heart valve also comprises a valvular structure disposed within the frame and configured to regulate the flow of blood through the frame in one direction and a plurality of actuation members each having a distal end coupled to the frame and configured to apply an axial force to the frame. The prosthetic heart valve is configured to self-expand from a radially compressed state to at least a partially radially expanded state. The medical assembly also comprises a delivery device. The delivery device comprises a plurality of delivery system actuators releasably coupled to the plurality of actuation members and extending through the plurality of channels of the frame and a plurality of support tubes disposed around the plurality of delivery system actuators and configured to allow the plurality of delivery system actuators to extend and move axially therethrough. One of a plurality of exterior surfaces of the plurality of delivery system actuators, a plurality of inner surfaces of the plurality of axially extending channels, or a plurality of inner surfaces of the plurality of support tubes comprise a plurality of resistance surfaces and the medical assembly further comprises a corresponding plurality of opposing surfaces that can engage the resistance surfaces, wherein a force resisting an axial movement of the delivery system actuators is increased when the resistance surface is engaged with the opposing surface.

When the prosthetic heart valve self-expands from the radially compressed state to the partially radially expanded state, the plurality of delivery system actuators can slide relative to the plurality of axially extending channels or the plurality of support tubes and the plurality of resistances surfaces slide against the opposing surfaces to control a rate of expansion of the prosthetic heart valve.

Example 55. The medical assembly of any example herein, particularly of example 54, wherein the delivery system actuators are configured to move the actuation members in an axial direction to further expand the prosthetic heart valve from the partially radially expanded state to a further radially expanded state.

Example 56. The medical assembly of any example herein, particularly of examples 54-55, wherein the delivery device comprises a sheath configured to retain the prosthetic heart valve in the radially compressed state and to deploy the prosthetic heart valve from the sheath to allow the prosthetic heart valve to self-expand.

Example 57. The medical assembly of any example herein, particularly of examples 54-55, wherein the delivery device comprises an adjustable loop circumferentially disposed around an exterior of the prosthetic heart valve to retain the prosthetic heart valve in the radially compressed state and to allow the prosthetic heart valve to self-expand when slack is introduced in the adjustable loop.

Example 58. The medical assembly of any of examples 54-57, wherein the resistance surfaces are configured to disengage from the opposing surfaces when the prosthetic heart valve reaches the partially radially expanded state.

Example 59. The medical assembly of any of examples 54-57, wherein the resistance surfaces are configured to disengage from the opposing surfaces when the prosthetic heart valve reaches a fully expanded state.

Example 60. The medical assembly of any of examples 54-59, wherein the frame comprises a proximal end portion and a distal end portion, wherein the proximal end portion comprises a plurality of frame apices, wherein the plurality of axially extending channels extend through the plurality of frame apices.

Example 61. The medical assembly of any example herein, particularly of example 60, wherein the actuation members are each configured to pass through a corresponding axially extending channel when the frame is fully expanded.

Example 62. The medical assembly of any example herein, particularly of examples 54-61, wherein the frame comprises at least one locking mechanism configured to engage the actuation members and prevent radial compression of the prosthetic heart valve after the prosthetic heart valve is expanded to the further radially expanded state.

Example 63. The medical assembly of any example herein, particularly of examples 54-62, wherein the frame is made of Nitinol.

Example 64. The medical assembly of any example herein, particularly of examples 54-63, wherein the resistance surfaces are on the delivery system actuators, and the opposing surfaces are on the inner surfaces of the axially extending channels.

Example 65. The medical assembly of any example herein, particularly of examples 54-63, wherein the resistance surfaces are on the delivery system actuators, and the opposing surfaces are on the inner surfaces of the support tubes.

Example 66. The medical assembly of any example herein, particularly of examples 54-63, wherein the resistance surfaces are on the inner surfaces of the axially extending channels and the opposing surfaces are on the delivery system actuators.

Example 67. T The medical assembly of any example herein, particularly of examples 54-63, wherein the resistance surfaces are on the inner surfaces of the support tubes and the opposing surfaces are on the delivery system actuators.

Example 68. The medical assembly of any example herein, particularly of examples 54-67, wherein the opposing surfaces are resistance surfaces.

Example 69. The medical assembly of any example herein, particularly of examples 54-68, wherein the resistance surfaces comprise a first set of roughened features and the opposing surfaces comprise a second set of roughened features, wherein the first set of roughened features is configured to mutually engage with the second set of roughened features and configured to slidably pass the second set of roughened features when the prosthetic heart valve is self-expanding.

Example 70. The medical assembly of any example herein, particularly of examples 54-68, wherein the resistance surfaces comprise a polymer.

Example 71. The medical assembly of any example herein, particularly of example 70, wherein the polymer is an elastomer.

Example 72. The medical assembly of any example herein, particularly of example 71, wherein the elastomer is silicone, flouroelastomer, perflouroelastomer, ethylene propylene, nitrile rubber, or a combination thereof.

Example 73. The medical assembly of any example herein, particularly of examples 54-68, wherein either the resistance surfaces or the opposing surfaces comprise a combination of teeth, roughened features, or polymer materials.

Example 74. The medical assembly of any example herein, particularly of examples 54-73, wherein the resistance surfaces are configured to slidably engage one or more other components of the frame and oppose axial motion of the delivery system actuators when the prosthetic heart valve is self-expanding.

Example 75. The medical assembly of any example herein, particularly of examples 54-73, wherein the resistance surfaces are configured to disengage completely from the frame and to provide no resistance to motion of the delivery system actuators when the prosthetic heart valve has self-expanded to the partially radially expanded state or to the further radially expanded state.

Example 76. The medical assembly of any example herein, particularly of examples 54-75, wherein the delivery system actuators are configured to be decoupled from the actuation members after the prosthetic heart valve is expanded to the further radially expanded state so that the delivery device can be removed from a patient's body.

Example 77. A delivery device, comprising at least one delivery system actuator, at least one support tube disposed around the delivery system actuator and configured to allow the delivery system actuator to extend and move axially therethrough. One of an exterior surface of the delivery system actuator or an interior surface of the support tube comprise a friction surface and the other of the exterior surface of the delivery system actuator comprises an opposing surface that engages the friction surface.

Example 78. The medical assembly of any example herein, particularly of example 77, wherein the delivery device comprises a sheath configured to retain a prosthetic heart valve in a radially compressed state and to deploy the prosthetic heart valve from the sheath to allow the prosthetic heart valve to self-expand.

Example 79. The medical assembly of any example herein, particularly of example 77, wherein the delivery device comprises an adjustable loop configured to be circumferentially disposed around an exterior of a prosthetic heart valve to retain the prosthetic heart valve in a radially compressed state and to allow the prosthetic heart valve to self-expand when slack is introduced in the adjustable loop.

Example 80. The medical assembly of any example herein, particularly of examples 77-79, wherein the delivery system actuator is configured to be releasably attached to a component of a self-expanding prosthetic heart valve frame, wherein expansion of the self-expanding prosthetic heart valve frame causes an axial motion of the delivery system actuator.

Example 81. The medical assembly of any example herein, particularly of example 80, wherein when the self-expanding prosthetic heart valve frame self-expands, the friction surface imparts a force on the delivery system actuator that resists the axial motion of the delivery system actuator and the self-expansion of the self-expanding prosthetic heart valve frame to control a rate of expansion of the self-expanding prosthetic heart valve frame.

Example 82. The medical assembly of any example herein, particularly of examples 77-81, wherein the friction surface is configured to disengage from the opposing surface during an implantation procedure.

Example 83. The medical assembly of any example herein, particularly of examples 77-82, wherein the friction surface is on the delivery system actuator and the opposing surface is on the interior surface of the support tube.

Example 84. The medical assembly of any example herein, particularly of examples 77-82, wherein the friction surface is on an inner surface of the support tube and the opposing surface is on the delivery system actuator.

Example 85. The delivery device of any of examples 77-84, wherein the opposing surface is a friction surface.

Example 86. The medical assembly of any example herein, particularly of examples 77-85, wherein the friction surface comprises a polymer.

Example 87. The medical assembly of any example herein, particularly of example 86, wherein the polymer is an elastomer.

Example 88. The medical assembly of any example herein, particularly of example 87, wherein the elastomer is silicone, flouroelastomer, perflouroelastomer, ethylene propylene, nitrile rubber, or a combination thereof.

Example 89. A method of implanting a radially expandable prosthetic heart valve, comprising inserting a distal end portion of a delivery device and a prosthetic heart valve into the vasculature of a patient, while the prosthetic heart valve is in a radially compressed state, positioning the prosthetic heart valve within or adjacent a desired implantation site and allowing the prosthetic heart valve to radially self-expand from the radially compressed state to at least a partially radially expanded state. When the prosthetic heart valve is self-expanding, a friction surface of the delivery device slidably engages an opposing surface to create sliding friction that opposes the self-expansion of the radially expandable prosthetic heart valve.

Example 90. The method of any example herein, particularly of example 89, wherein the method further comprises actuating an actuator of the delivery device to further radially expand the prosthetic heart valve to a fully-expanded state.

Example 91. The method of any example herein, particularly of example 90, wherein the friction surface disengages the opposing surface when the prosthetic heart valve has self-expanded to the partially radially expanded state.

Example 92. The method of any example herein, particularly of example 89, wherein the method further comprises allowing the prosthetic heart valve to self-expand from the partially radially expanded state to a further radially expanded state.

Example 93. The method of any example herein, particularly of example 92, wherein the friction surface disengages the opposing surface when the prosthetic heart valve has self-expanded to the further radially expanded state.

Example 94. T The method of any example herein, particularly of examples 89-93, wherein the method further comprises locking the radially expandable prosthetic heart valve in a fully the further radially expanded state.

Example 95. The method of any example herein, particularly of examples 89-94, wherein the friction surface is disposed on a delivery system actuator of the delivery device and the opposing surface is disposed on a component of the prosthetic heart valve.

Example 96. The method of any example herein, particularly of examples 89-94, wherein the friction surface is disposed on a delivery system actuator of the delivery device and the opposing surface is disposed on another component of the delivery device.

Example 97. The method of any example herein, particularly of examples 89-94, wherein the opposing surface is a friction surface.

Example 98. The method of any example herein, particularly of examples 89-97, wherein the friction surface comprises a polymer.

Example 99. The method of any example herein, particularly of example 98, wherein the polymer is an elastomer.

Example 100. The method of any example herein, particularly of example 99, wherein the elastomer is silicone, flouroelastomer, perflouroelastomer, ethylene propylene, nitrile rubber, or a combination thereof.

Example 101. A medical assembly comprising a prosthetic heart valve. The prosthetic heart valve comprises a radially expandable annular frame, wherein the frame comprises at least one frame portion defining an axially extending channel, a valvular structure disposed within the frame and configured to regulate the flow of blood through the frame in one direction, at least one actuation member having a distal end coupled to the frame and configured to apply an axial force to the frame. The prosthetic heart valve is configured to self-expand from a radially compressed state to at least a partially radially expanded state. The medical assembly also comprises a delivery device. The delivery device comprises at least one delivery system actuator releasably coupled to the actuation member and extending through the axially extending channel of the frame. One of an exterior surface of the delivery system actuator and an inner surface the axially extending channel comprises a friction surface and the other of the exterior surface of the delivery system actuator and the inner surface of the axially extending channel comprises an opposing surface that can engage the friction surface. A coefficient of friction between the delivery system actuator and the axially extending channel is increased when the friction surface is engaged with the opposing surface. The frame has an elastic radial expansion force when in the radially compressed state that causes the prosthetic heart valve to self-expand from the radially compressed state to the partially radially expanded state, and the friction surface partially opposes the elastic radial expansion force to permit self-expansion of the frame at a rate lower than a free expansion rate. The delivery system actuator is configured to move the actuation member in an axial direction to further expand the prosthetic heart valve from the partially radially expanded state to a further radially expanded state.

Example 102. The medical assembly of any example herein, particularly of example 101, wherein the delivery device comprises a sheath configured to retain the prosthetic heart valve in the radially compressed state and to deploy the prosthetic heart valve from the sheath to allow the prosthetic heart valve to self-expand.

Example 103. The medical assembly of any example herein, particularly of example 101, wherein the delivery device comprising an adjustable loop circumferentially disposed around an exterior of the prosthetic heart valve to retain the prosthetic heart valve in the radially compressed state and to allow the prosthetic heart valve to self-expand when slack is introduced in the adjustable loop.

Example 104. The medical assembly of any example herein, particularly of examples 101-103, wherein the friction surface is configured to disengage from the opposing surface when the prosthetic heart valve reaches the partially radially expanded state.

Example 105. The medical assembly of any example herein, particularly of examples 101-104, wherein the frame comprises a proximal end portion and a distal end portion, wherein the proximal end portion comprises a plurality of frame apices, wherein the axially extending channel extends through one of the frame apices.

Example 106. The medical assembly of any example herein, particularly of example 105, wherein the actuation member is configured to pass through the axially extending channel when the frame is fully expanded.

Example 107. The medical assembly of any example herein, particularly of examples 101-106, wherein the frame comprises at least one locking member configured to engage the actuation member and prevent radial compression of the prosthetic heart valve after the prosthetic heart valve is expanded to the further radially expanded state.

Example 108. The medical assembly of any example herein, particularly of examples 101-107, wherein the radially expandable annular frame is made of Nitinol.

Example 109. The medical assembly of any example herein, particularly of examples 101-108, wherein the friction surface is on the delivery system actuator and the opposing surface is on the inner surface of the axially extending channel.

Example 110. The medical assembly of any example herein, particularly of examples 101-108, wherein the friction surface is on the inner surface of the axially extending channel and the opposing surface is on the delivery system actuator.

Example 111. The medical assembly of any example herein, particularly of examples 101-108, wherein the opposing surface comprises a friction surface.

Example 112. The medical assembly of any example herein, particularly of examples 101-110, wherein the friction surface comprises a polymer.

Example 113. The medical assembly of any example herein, particularly of example 112, wherein the polymer is an elastomer.

Example 114. The medical assembly of any example herein, particularly of example 113, wherein the elastomer is silicone, flouroelastomer, perflouroelastomer, ethylene propylene, nitrile rubber, or a combination thereof.

Example 115. The medical assembly of any example herein, particularly of examples 101-114 wherein the friction surface is configured to disengage completely from the radially expandable annular frame and to provide no resistance to axial motion of the delivery system actuator when the prosthetic heart valve has self-expanded to the partially radially expanded state.

Example 116. T The medical assembly of any example herein, particularly of examples 101-115, wherein the at least one actuation member comprises a plurality of actuation members and the at least one delivery system actuator comprises a plurality of delivery system actuators releasably coupled to respective actuation members and extending through respective channels of the radially expandable annular frame.

Example 117. The medical assembly of any example herein, particularly of example 116, wherein each delivery system actuator and corresponding channel has a friction surface and an opposing surface.

Example 118. The medical assembly of any example herein, particularly of examples 116-117, wherein the delivery device comprises a plurality of support tubes that abut the radially expandable annular frame, wherein each delivery system actuator extends through one of the support tubes.

Example 119. The medical assembly of any example herein, particularly of examples 101-118, wherein the delivery system actuator is configured to be de-coupled from the actuation member after the prosthetic heart valve is expanded to the further radially expanded state so that the delivery device can be removed from a patient's body.

Example 120. A prosthetic heart valve comprising a radially expandable annular frame, a valvular structure disposed within the frame and configured to regulate flow of blood through the frame in one direction, at least one actuation member rotatably coupled to the frame and comprising an actuation member aperture, and a flexible tension member extending through the actuation member aperture and around the frame.

Example 121. The prosthetic heart valve of any example herein, particularly of example 120, wherein the prosthetic heart valve is configured to self-expand from a radially compressed state to a radially expanded.

Example 122. The prosthetic heart valve of any example herein, particularly of example 121, wherein the flexible tension member forms at least one local loop around the actuation member in the compressed state.

Example 123. The prosthetic heart valve of any example herein, particularly of example 121 or 122, wherein, when the prosthetic heart valve is in the compressed state and the actuation member is rotated in a first rotational direction, a radially inwardly directed force applied to the frame by the flexible tension member is gradually lessened such that the frame can expand at a controlled rate to a selected diameter.

Example 124. The prosthetic heart valve of any example herein, particularly of any one of examples 120-123, wherein the at least one actuation member comprises a plurality of actuation members and wherein the flexible tension member extends through the actuation member aperture of at least one of the actuation members.

Example 125. The prosthetic heart valve of any example herein, particularly of example 124, wherein the flexible tension member extends through the actuation member apertures of all of the actuation members.

Example 126. The prosthetic heart valve of any example herein, particularly of any one of examples 120-125, wherein the at least one actuation member comprises a rigid rod.

Example 127. The prosthetic heart valve of any example herein, particularly of any one of examples 120-126, wherein the frame is made of Nitinol.

Example 128. The prosthetic heart valve of any example herein, particularly of any one of examples 121-127, wherein, when depending on example 121, the prosthetic heart valve is configured to self-expand from the compressed state to a selected radially expanded state.

Example 129. The prosthetic heart valve of any example herein, particularly of any one of examples 120-128, wherein the actuation member aperture is disposed between an inflow end and an outflow end of the prosthetic heart valve.

Example 130. The prosthetic heart valve of any example herein, particularly of example 129, wherein the actuation member aperture is disposed halfway between an inflow end and an outflow end.

Example 131. The prosthetic heart valve of any example herein, particularly of example 129, wherein the actuation member aperture is closer to the inflow end than to the outflow end.

Example 132. The prosthetic heart valve of any example herein, particularly of example 129, wherein the actuation member aperture is closer to the outflow end than to the inflow end.

Example 133. The prosthetic heart valve of any example herein, particularly of any one of examples 120-132, wherein the flexible tension member forms a single loop encircling the frame.

Example 134. The prosthetic heart valve of any example herein, particularly of any one of examples 120-132, wherein the flexible tension member spans a distance less than a full circumference of the frame.

Example 135. The prosthetic heart valve of any example herein, particularly of any one of examples 120-134, wherein the flexible tension member is weaved through cells of the frame.

Example 136. A medical assembly comprising a prosthetic heart valve. The prosthetic heart valve comprises a radially expandable annular frame, a valvular structure disposed within the frame and configured to regulate flow of blood through the frame in one direction, at least one actuation member rotatably coupled to the frame and comprising an actuation member aperture, and a flexible tension member extending through the actuation member aperture and around the frame. The medical assembly further comprises a delivery device. The delivery device comprises at least one delivery system actuator.

Example 137. The medical assembly valve of any example herein, particularly of example 136, wherein the delivery system actuator is configured to releasably couple to the actuation member.

Example 138. The medical assembly valve of any example herein, particularly of example 136 or 137, wherein rotational movement of the delivery system actuator is configured to apply rotational movement of the actuation member.

Example 139. The medical assembly valve of any example herein, particularly any one of examples 136-138, wherein the prosthetic heart valve is configured to self-expand from a radially compressed state to a radially expanded state.

Example 140. The medical assembly valve of any example herein, particularly of example 139, wherein the flexible tension member forms at least one local loop around the actuation member in the compressed state.

Example 141. The medical assembly valve of any example herein, particularly of example 139 or 140, wherein, when the prosthetic heart valve is in the compressed state and the delivery system actuator is rotated in a first rotational direction to rotate the actuation member therewith, a radially inwardly directed force applied to the frame by the flexible tension member is gradually lessened such that the frame can expand at a controlled rate to a selected diameter.

Example 142. The medical assembly valve of any example herein, particularly of any one of examples 139-141, wherein the delivery device comprises a sheath configured to retain the prosthetic heart valve in the radially compressed state.

Example 143. The medical assembly valve of any example herein, particularly of example 142, wherein the delivery device is further configured to deploy the prosthetic heart valve from the sheath to allow the prosthetic heart valve to self-expand to a diameter defined by the flexible tension member.

Example 130. The medical assembly valve of any example herein, particularly any one of examples 136-143, wherein the at least one actuation member comprises a plurality of actuation members and wherein the flexible tension member extends through the actuation member aperture of at least one of the actuation members

Example 140. The medical assembly valve of any example herein, particularly of example 144, wherein the flexible tension member extends through the actuation member apertures of all of the actuation members.

Example 146. The medical assembly valve of any example herein, particularly of example 144 or 145, wherein the at least one delivery system actuator comprises a plurality of delivery system actuators releasably coupled to respective actuation members.

Example 147. The medical assembly valve of any example herein, particularly any one of examples 136-146, wherein the at least one actuation member comprises a rigid rod.

Example 148. The medical assembly valve of any example herein, particularly any one of examples 136-147, wherein the frame is made of Nitinol.

Example 149. The medical assembly valve of any example herein, particularly any one of examples 139-148, wherein, when depending on example 139, the prosthetic heart valve is configured to self-expand from the compressed state to a selected radially expanded state.

Example 150. The medical assembly valve of any example herein, particularly of example 149, wherein the delivery system actuator is configured to be de-coupled from the actuation member after the prosthetic heart valve is expanded to the selected radially expanded state so that the delivery device can be retrieved away from the prosthetic heart valve.

Example 151. The medical assembly valve of any example herein, particularly any one of examples 136-150, wherein the actuation member aperture is disposed between an inflow end and an outflow end of the prosthetic heart valve.

Example 152. The medical assembly valve of any example herein, particularly of example 151, wherein the actuation member aperture is disposed halfway between an inflow end and an outflow end.

Example 153. The medical assembly valve of any example herein, particularly of example 151, wherein the actuation member aperture is closer to the inflow end than to the outflow end.

Example 154. The medical assembly valve of any example herein, particularly of example 151, wherein the actuation member aperture is closer to the outflow end than to the inflow end.

Example 155. The medical assembly valve of any example herein, particularly any one of examples 136-154, wherein the flexible tension member forms a single loop encircling the frame.

Example 156. The medical assembly valve of any example herein, particularly any one of examples 136-154, wherein the flexible tension member spans a distance less than a full circumference of the frame.

Example 157. The medical assembly valve of any example herein, particularly any one of examples 136-156, wherein the flexible tension member is weaved through cells of the frame.

Example 158. A prosthetic heart valve comprising a radially expandable annular frame, a valvular structure disposed within the frame and configured to regulate flow of blood through the frame in one direction, at least one actuation member rotatably coupled to the frame and comprising a terminal end and an actuation member slot extending from the terminal end, and a flexible tension member extending through the actuation member slot and around the frame.

Example 159. The prosthetic heart valve of any example herein, particularly of example 158, wherein the prosthetic heart valve is configured to self-expand from a radially compressed state to a radially expanded.

Example 160. The prosthetic heart valve of any example herein, particularly of example 159, wherein the flexible tension member forms at least one local loop around the actuation member in the compressed state.

Example 161. The prosthetic heart valve of any example herein, particularly of example 159 or 160, wherein, when the prosthetic heart valve is in the compressed state and the actuation member is rotated in a first rotational direction, a radially inwardly directed force applied to the frame by the flexible tension member is gradually lessened such that the frame can expand at a controlled rate to a selected diameter.

Example 162. The prosthetic heart valve of any example herein, particularly of any one of examples 158-161, wherein the at least one actuation member comprises a plurality of actuation members and wherein the flexible tension member extends through the actuation member slot of at least one of the actuation members.

Example 163. The prosthetic heart valve of any example herein, particularly of example 162, wherein the flexible tension member extends through the actuation member slots of all of the actuation members.

Example 164. The prosthetic heart valve of any example herein, particularly of any one of example 158-163, wherein the at least one actuation member comprises a rigid rod.

Example 165. The prosthetic heart valve of any example herein, particularly of any one of examples 158-164, wherein the frame is made of Nitinol.

Example 166. The prosthetic heart valve of any example herein, particularly of any one of examples 158-165, wherein, when depending on example 159, the prosthetic heart valve is configured to self-expand from the compressed state to a selected radially expanded state.

Example 167. The prosthetic heart valve of any example herein, particularly of any one of examples 158-166, wherein the actuation member slot is disposed between an inflow end and an outflow end of the prosthetic heart valve.

Example 168. The prosthetic heart valve of any example herein, particularly of example 167, wherein the actuation member slot is disposed halfway between an inflow end and an outflow end.

Example 169. The prosthetic heart valve of any example herein, particularly of example 167, wherein the actuation member slot is closer to the inflow end than to the outflow end.

Example 170. The prosthetic heart valve of any example herein, particularly of example 167, wherein the actuation member slot is closer to the outflow end than to the inflow end.

Example 171. The prosthetic heart valve of any example herein, particularly of any one of examples 158-170, wherein the flexible tension member forms a single loop encircling the frame.

Example 172. The prosthetic heart valve of any example herein, particularly of any one of examples 158-170, wherein the flexible tension member spans a distance less than a full circumference of the frame.

Example 173. The prosthetic heart valve of any example herein, particularly of any one of examples 158-171, wherein the flexible tension member is weaved through cells of the frame.

Example 174. The prosthetic heart valve of any example herein, particularly of any one of examples 158-173, wherein the actuation member is axially movable relative to the frame.

Example 175. The prosthetic heart valve of any example herein, particularly of example 174, wherein the actuation member is configured to releasably couple to the frame, and to be de-coupled from the frame by being axially pulled in a manner that allows the flexible tension member to slide out of the actuation member slot.

Example 176. A medical assembly comprising a prosthetic heart valve. The prosthetic heart valve comprises a radially expandable annular frame, a valvular structure disposed within the frame and configured to regulate flow of blood through the frame in one direction, and a flexible tension member extending around the frame. The medical assembly further comprises a delivery device. The delivery device comprises at least one delivery system actuator comprising a terminal end and an actuation member slot extending from the terminal end.

Example 177. The medical assembly of any example herein, particularly of example 176, wherein the delivery system actuator is configured to releasably couple to the frame.

Example 178. The medical assembly of any example herein, particularly of example 176 or 177, wherein the delivery system actuator is rotatably and axially movable with respect to the frame.

Example 179. The medical assembly of any example herein, particularly of any one of examples 176-178, wherein the prosthetic heart valve is configured to self-expand from a radially compressed state to a radially expanded state.

Example 180. The medical assembly of any example herein, particularly of example 179, wherein the flexible tension member forms at least one local loop around the delivery system actuator in the compressed state.

Example 181. The medical assembly of any example herein, particularly of example 179 or 180, wherein, when the prosthetic heart valve is in the compressed state and the delivery system actuator is rotated in a first rotational direction, a radially inwardly directed force applied to the frame by the flexible tension member is gradually lessened such that the frame can expand at a controlled rate to a selected diameter.

Example 182. The medical assembly of any example herein, particularly of any one of examples 179-181, wherein the delivery device comprises a sheath configured to retain the prosthetic heart valve in the radially compressed state.

Example 183. The medical assembly of any example herein, particularly of example 182, wherein the delivery device is further configured to deploy the prosthetic heart valve from the sheath to allow the prosthetic heart valve to self-expand to a diameter defined by the flexible tension member.

Example 184. The medical assembly of any example herein, particularly of any one of examples 176-183, wherein the at least one delivery system actuator comprises a plurality of delivery system actuators and wherein the flexible tension member extends through the actuation member slot of at least one of the delivery system actuators.

Example 185. The medical assembly of any example herein, particularly of example 184, wherein the flexible tension member extends through the actuation member slots of all of the delivery system actuators.

Example 186. The medical assembly of any example herein, particularly of any one of examples 176-185, wherein the frame is made of Nitinol.

Example 187. The medical assembly of any example herein, particularly of any one of examples 179-186, wherein, when depending on example 179, the prosthetic heart valve is configured to self-expand from the compressed state to a selected radially expanded state.

Example 188. The medical assembly of any example herein, particularly of example 187, wherein the delivery system actuator is configured to be de-coupled from the frame after the prosthetic heart valve is expanded to the selected radially expanded state by being axially pulled in a manner that allows the flexible tension member to slide out of the actuation member slot, so that the delivery device can be retrieved away from the prosthetic heart valve.

Example 189. The medical assembly of any example herein, particularly of any one of examples 176-188, wherein the actuation member slot is disposed between an inflow end and an outflow end of the prosthetic heart valve.

Example 190. The medical assembly of any example herein, particularly of example 189, wherein the actuation member slot is disposed halfway between an inflow end and an outflow end.

Example 191. The medical assembly of any example herein, particularly of example 189, wherein the actuation member slot is closer to the inflow end than to the outflow end.

Example 192. The medical assembly of any example herein, particularly of example 189, wherein the actuation member slot is closer to the outflow end than to the inflow end.

Example 193. The medical assembly of any example herein, particularly of any one of examples 176-192, wherein the flexible tension member forms a single loop encircling the frame.

Example 194. The medical assembly of any example herein, particularly of any one of examples 176-192, wherein the flexible tension member spans a distance less than a full circumference of the frame.

Example 195. The medical assembly of any example herein, particularly of any one of examples 176-193, wherein the flexible tension member is weaved through cells of the frame.

Example 196. A medical assembly comprising a prosthetic heart valve. The prosthetic heart valve comprises a radially expandable annular frame, a valvular structure disposed within the frame and configured to regulate flow of blood through the frame in one direction, and a flexible tension member. The frame comprises at least one non-commissural vertical post that comprises a channel extending therethrough between an aperture and a proximal end thereof. The flexible tension members extends around the frame and forms a local loop that extends through the channel. The medical assembly further comprises a delivery device. The delivery device comprises a delivery system shaft defining a lumen, and a resistance feature disposed within the lumen and defining an internal passage that comprises a friction surface.

Example 197. The medical assembly of any example herein, particularly of example 196, wherein the local loop defines a first portion and a second portion that extend through the channel.

Example 198. The medical assembly of any example herein, particularly of example 196 or 197, wherein the prosthetic heart valve is configured to self-expand from a radially compressed state to a radially expanded state.

Example 199. The medical assembly of any example herein, particularly of example 198, wherein the local loop extends from the channel into the internal passage and is frictionally engaged with the friction surface of the resistance feature in the compressed state.

Example 200. The medical assembly of any example herein, particularly of example 199, wherein, when the prosthetic heart valve self-expands from the compressed state to the expanded state, the local loop is configured to slide relative to the resistance feature such that a radially inwardly directed force applied to the frame by the flexible tension member is gradually lessened and the frame can expand at a controlled rate to a selected diameter.

Example 201. The medical assembly of any example herein, particularly of any one of examples 196-200, wherein the internal passage is an axially extending central channel formed through the resistance feature.

Example 202. The medical assembly of any example herein, particularly of any one of examples 196-200, wherein the resistance feature defines an outer cutout face distanced from an inner surface of the delivery system shaft, and wherein the internal passage is defined between the outer cutout face of the resistance feature and the inner surface of the delivery system shaft.

Example 203. The medical assembly of any example herein, particularly of any one of examples 196-202, wherein the delivery system shaft is configured to releasably couple to the non-commissural vertical post.

Example 204. The medical assembly of any example herein, particularly of any one of examples 196-203, wherein a first coefficient of friction exists between the flexible tension member and the channel, wherein a second coefficient of friction exists between the flexible tension member and the resistance feature, and wherein the first and the second coefficients of friction are different from each other.

Example 205. The medical assembly of any example herein, particularly of example 204, wherein the second coefficient of friction is higher than the first coefficient of friction.

Example 206. The medical assembly of any example herein, particularly of any one of examples 196-205, wherein the friction surface comprises a polymer.

Example 207. The medical assembly of any example herein, particularly of example 206, wherein the polymer is an elastomer.

Example 208. The medical assembly of any example herein, particularly of example 207, wherein the elastomer is silicone, flouroelastomer, perflouroelastomer, ethylene propylene, nitrile rubber, or a combination thereof.

Example 209. The medical assembly of any example herein, particularly of any one of examples 196-208, wherein the frame is made of Nitinol.

Example 210. The medical assembly of any example herein, particularly of any one of examples 196-209, wherein the flexible tension member forms a single loop encircling the frame.

Example 211. The medical assembly of any example herein, particularly of any one of examples 196-209, wherein the flexible tension member spans a distance less than a full circumference of the frame.

Example 212. The medical assembly of any example herein, particularly of any one of examples 196-211, wherein the flexible tension member is weaved through cells of the frame.

Example 213. The medical assembly of any example herein, particularly of any one of examples 196-212, wherein the prosthetic heart valve further comprises an outer skirt disposed around and attached to the frame, wherein the outer skirt comprises a sleeve and an opening, and wherein the flexible tension member extends out of the aperture of the non-commissural vertical post, through the opening, into the sleeve.

Example 214. The medical assembly of any example herein, particularly of example 213, wherein the sleeve is positioned halfway between an inflow end and an outflow end of the prosthetic heart valve.

Example 215. The medical assembly of any example herein, particularly of example 214, wherein the sleeve is positioned halfway between an inflow end and an outflow end.

Example 216. The medical assembly of any example herein, particularly of example 214, wherein the sleeve is closer to the inflow end than to the outflow end.

Example 217. The medical assembly of any example herein, particularly of example 214, wherein the sleeve is closer to the outflow end than to the inflow end.

Example 218. The medical assembly of any example herein, particularly of any one of examples 196-217, wherein the frame is made of Nitinol.

Example 219. The medical assembly of any example herein, particularly of any one of examples 198-218, wherein, when depending on example 198, the prosthetic heart valve is configured to self-expand from the compressed state to a partially radially expanded state, and wherein the local loop is frictionally engaged with the friction surface of the resistance feature when the prosthetic heart valve self-expands from the compressed state to the partially radially expanded state.

Example 220. The medical assembly of any example herein, particularly of example 219, wherein the flexible tension member is configured to disengage from the resistance feature when the prosthetic heart valve reaches the partially radially expanded state.

Example 221. The medical assembly of any example herein, particularly of example 220, wherein the prosthetic valve further comprises a plurality of actuation members coupled to the frame and configured to apply an axially directed force to the frame, wherein the delivery device further comprises a plurality of delivery system actuators releasably coupled to the respective actuation members, and wherein the delivery system actuators are configured to move the actuation members in an axial direction to further expand the prosthetic heart valve from the partially radially expanded state to a further radially expanded state.

Example 222. The medical assembly of any example herein, particularly of any one of examples 198-218, wherein, when depending on example 198, the prosthetic heart valve is configured to self-expand from the compressed state to a fully radially expanded state, and wherein the local loop is frictionally engaged with the friction surface of the resistance feature when the prosthetic heart valve self-expands from the compressed state to the fully radially expanded state.

Example 223. A medical assembly comprising a prosthetic heart valve. The prosthetic heart valve comprises a radially expandable annular frame, a valvular structure disposed within the frame and configured to regulate flow of blood through the frame in one direction, and a flexible tension member extending around the frame and comprising a first end portion coupled to the frame, and a second end portion opposite to the first end portion. The medical assembly further comprises a delivery device. The delivery device comprises a delivery system shaft defining a lumen, and a resistance feature disposed within the lumen and defining an internal passage that comprises a friction surface.

Example 224. The medical assembly of any example herein, particularly of example 223, wherein the prosthetic heart valve is configured to self-expand from a radially compressed state to a radially expanded state.

Example 225. The medical assembly of any example herein, particularly of example 224, wherein the second end portion extends into the internal passage and is frictionally engaged with the friction surface of the resistance feature in the compressed state.

Example 226. The medical assembly of any example herein, particularly of example 225, wherein, when the prosthetic heart valve self-expands from the compressed state to the expanded state, the second end portion is configured slide relative to the resistance feature such that a radially inwardly directed force applied to the frame by the flexible tension member is gradually lessened and the frame can expand at a controlled rate to a selected diameter.

Example 227. The medical assembly of any example herein, particularly of any one of examples 223-226, wherein the internal passage is an axially extending central channel formed through the resistance feature.

Example 228. The medical assembly of any example herein, particularly of any one of examples 223-226, wherein the resistance feature defines an outer cutout face distanced from an inner surface of the delivery system shaft, and wherein the internal passage is defined between the outer cutout face of the resistance feature and the inner surface of the delivery system shaft.

Example 229. The medical assembly of any example herein, particularly of any one of examples 223-228, wherein the flexible tension member extends around the frame in a continuous helical manner defining a plurality of loops.

Example 230. The medical assembly of any example herein, particularly of any one of examples 223-228, wherein the flexible tension member forms a single loop encircling the frame.

Example 231. The medical assembly of any example herein, particularly of any one of examples 223-228, wherein the flexible tension member extends around a portion of the frame which is less than a full circumference of the frame.

Example 232. The medical assembly of any example herein, particularly of any one of examples 223-231, wherein the first end portion comprises a knot tying the flexible tension member to the frame.

Example 233. The medical assembly of any example herein, particularly of any one of examples 223-231, wherein a first coefficient of friction exists between the flexible tension member and the frame, wherein a second coefficient of friction exists between the flexible tension member and the resistance feature, and wherein the first and the second coefficients of friction are different from each other.

Example 234. The medical assembly of any example herein, particularly of example 233, wherein the second coefficient of friction is higher than the first coefficient of friction.

Example 235. The medical assembly of any example herein, particularly of any one of examples 223-234, wherein the friction surface comprises a polymer.

Example 236. The medical assembly of any example herein, particularly of example 235, wherein the polymer is an elastomer.

Example 237. The medical assembly of any example herein, particularly of example 236, wherein the elastomer is silicone, flouroelastomer, perflouroelastomer, ethylene propylene, nitrile rubber, or a combination thereof.

Example 238. The medical assembly of any example herein, particularly of any one of examples 223-237, wherein the frame is made of Nitinol.

Example 239. The medical assembly of any example herein, particularly of any one of examples 223-238, wherein the flexible tension member is weaved through cells of the frame.

Example 240. The medical assembly of any example herein, particularly of any one of examples 224-239, wherein, when depending on example 224, the prosthetic heart valve is configured to self-expand from the compressed state to a partially radially expanded state, and wherein the second end portion is frictionally engaged with the friction surface of the resistance feature when the prosthetic heart valve self-expands from the compressed state to the partially radially expanded state.

Example 241. The medical assembly of any example herein, particularly of example 240, wherein the flexible tension member is configured to disengage from the resistance feature when the prosthetic heart valve reaches the partially radially expanded state.

Example 242. The medical assembly of any example herein, particularly of example 241, wherein the prosthetic valve further comprises a plurality of actuation members coupled to the frame and configured to apply an axially directed force to the frame, wherein the delivery device further comprises a plurality of delivery system actuators releasably coupled to the respective actuation members, and wherein the delivery system actuators are configured to move the actuation members in an axial direction to further expand the prosthetic heart valve from the partially radially expanded state to a further radially expanded state.

Example 243. The medical assembly of any example herein, particularly of any one of examples 224-239, wherein, when depending on example 224, the prosthetic heart valve is configured to self-expand from the compressed state to a fully radially expanded state, and wherein the second end portion is frictionally engaged with the friction surface of the resistance feature when the prosthetic heart valve self-expands from the compressed state to the fully radially expanded state.

Example 244. A medical assembly comprising a prosthetic heart valve. The prosthetic heart valve comprises a radially expandable annular frame, a valvular structure disposed within the frame and configured to regulate flow of blood through the frame in one direction, and a flexible tension member extending around the frame. The medical assembly further comprises a delivery device. The delivery device comprises a delivery system shaft defining a lumen, and a resistance feature disposed within the lumen and defining an internal passage that comprises a friction surface. At least a portion of the flexible tension member is configured to slide through the internal passage during radial expansion of the frame

Example 245. The medical assembly of any example herein, particularly of example 244, wherein the prosthetic heart valve is configured to self-expand from a radially compressed state to a radially expanded state.

Example 246. The medical assembly of any example herein, particularly of example 244 or 245, wherein the frame comprises at least one non-commissural vertical post that comprises a channel extending therethrough between an aperture and a proximal end thereof, and wherein the flexible tension member forms a local loop that extends through the channel.

Example 247. The medical assembly of any example herein, particularly of example 246, wherein, when depending on example 245, the local loop extends from the channel into the internal passage and is frictionally engaged with the friction surface of the resistance feature in the compressed state.

Example 248. The medical assembly of any example herein, particularly of example 247, wherein, when the prosthetic heart valve self-expands from the compressed state to the expanded state, the local loop is configured to slide relative to the resistance feature such that a radially inwardly directed force applied to the frame by the flexible tension member is gradually lessened and the frame can expand at a controlled rate to a selected diameter.

Example 249. The medical assembly of any example herein, particularly of any one of examples 246-248, wherein a first coefficient of friction exists between the flexible tension member and the channel, wherein a second coefficient of friction exists between the flexible tension member and the resistance feature, and wherein the first and the second coefficients of friction are different from each other.

Example 250. The medical assembly of any example herein, particularly of example 244 or 245, wherein the flexible tension member comprises a first end portion coupled to the frame, and a second end portion opposite to the first end portion.

Example 251. The medical assembly of any example herein, particularly of example 250, wherein, when depending on example 245, the second end portion extends into the internal passage and is frictionally engaged with the friction surface of the resistance feature in the compressed state.

Example 252. The medical assembly of any example herein, particularly of example 251, wherein, when the prosthetic heart valve self-expands from the compressed state to the expanded state, the second end portion is configured slide relative to the resistance feature such that a radially inwardly directed force applied to the frame by the flexible tension member is gradually lessened and the frame can expand at a controlled rate to a selected diameter.

Example 253. The medical assembly of any example herein, particularly of any one of examples 250-252, wherein a first coefficient of friction exists between the flexible tension member and the frame, wherein a second coefficient of friction exists between the flexible tension member and the resistance feature, and wherein the first and the second coefficients of friction are different from each other.

Example 254. The medical assembly of any example herein, particularly of example 249 or 253, wherein the second coefficient of friction is higher than the first coefficient of friction.

In view of the many possible ways to which the principles of the disclosed invention may be applied, it should be recognized that what is disclosed herein are only preferred examples of the invention and should not be taken as limiting the scope of the invention. Rather, the scope of the invention is defined by the following claims. We therefore claim as our invention all that comes within the scope and spirit of these claims.

1. A medical assembly comprising:
a prosthetic heart valve, comprising:
a radially expandable annular frame, wherein the frame comprises at least one frame portion defining an axially extending channel;
a valvular structure disposed within the frame and configured to regulate flow of blood through the frame in one direction;
at least one actuation member coupled to the frame and configured to apply an axially directed force to the frame;
wherein the prosthetic heart valve is configured to self-expand from a radially compressed state to at least a partially radially expanded state;
a delivery device comprising:
at least one delivery system actuator releasably coupled to the actuation member and extending through the axially extending channel of the frame;
wherein one of an exterior surface of the delivery system actuator and an inner surface the axially extending channel comprises a friction surface and the other of the exterior surface of the delivery system actuator and the inner surface of the axially extending channel comprises an opposing surface that can engage the friction surface, wherein the exterior surface of the delivery system actuator and the inner surface of the axially extending channel have a first coefficient of friction when the friction surface and the opposing surface engage each other and a second coefficient of friction when the friction surface and the opposing surface do not engage each other, wherein the first coefficient of friction is greater than the second coefficient of friction;
wherein, when the prosthetic heart valve self-expands from the radially compressed state to the partially radially expanded state, the delivery system actuator can slide relative to the axially extending channel and the friction surface slides against the opposing surface to control a rate of expansion of the prosthetic heart valve;
wherein the delivery system actuator is configured to move the actuation member in an axial direction to further expand the prosthetic heart valve from the partially radially expanded state to a further radially expanded state.

2. The medical assembly of claim 1, wherein the friction surface is configured to disengage from the opposing surface when the prosthetic heart valve reaches the partially radially expanded state.

3. The medical assembly of claim 1, wherein the frame comprises a proximal end portion and a distal end portion, wherein the proximal end portion comprises a plurality of frame apices, wherein the axially extending channel extends through one of the frame apices.

4. The medical assembly of claim 1, wherein the friction surface is on the delivery system actuator and the opposing surface is on the inner surface of the axially extending channel.

5. The medical assembly of claim 1, wherein the friction surface is on the inner surface of the axially extending channel and the opposing surface is on the delivery system actuator.

6. The medical assembly of claim 1 wherein the friction surface is configured to disengage completely from the frame and to provide no resistance to axial motion of the delivery system actuator when the prosthetic heart valve has self-expanded to the partially radially expanded state.

7. A prosthetic heart valve comprising:
a radially expandable annular frame;
a valvular structure disposed within the frame and configured to regulate flow of blood through the frame in one direction;
at least one actuation member rotatably coupled to the frame and comprising an actuation member aperture; and
a flexible tension member extending through the actuation member aperture and around the frame.

8. The prosthetic heart valve of claim 7, wherein the prosthetic heart valve is configured to self-expand from a radially compressed state to a radially expanded.

9. The prosthetic heart valve of claim 8, wherein the flexible tension member forms at least one local loop around the actuation member in the compressed state.

10. The prosthetic heart valve of claim 7, wherein, when the prosthetic heart valve is in the compressed state and the actuation member is rotated in a first rotational direction, a radially inwardly directed force applied to the frame by the flexible tension member is gradually lessened such that the frame can expand at a controlled rate to a selected diameter.

11. The prosthetic heart valve of claim 7, wherein the at least one actuation member comprises a rigid rod.

12. A medical assembly comprising:
a prosthetic heart valve comprising:
a radially expandable annular frame;
a valvular structure disposed within the frame; and configured to regulate flow of blood through the frame in one direction;
a flexible tension member extending around the frame; and
a delivery device comprising:
a delivery system shaft defining a lumen; and
a resistance feature disposed within the lumen and defining an internal passage that comprises a friction surface;
wherein at least a portion of the flexible tension member is configured to slide through the internal passage during radial expansion of the frame.

13. The medical assembly of claim 12, wherein the prosthetic heart valve is configured to self-expand from a radially compressed state to a radially expanded state.

14. The medical assembly of claim 12, wherein the frame comprises at least one non-commissural vertical post that comprises a channel extending therethrough between an aperture and a proximal end thereof, and wherein the flexible tension member forms a local loop that extends through the channel.

15. The medical assembly of claim 14, wherein, when depending on claim 13, the local loop extends from the channel into the internal passage and is frictionally engaged with the friction surface of the resistance feature in the compressed state.

16. The medical assembly of claim 15, wherein, when the prosthetic heart valve self-expands from the compressed state to the expanded state, the local loop is configured to slide relative to the resistance feature such that a radially inwardly directed force applied to the frame by the flexible tension member is gradually lessened and the frame can expand at a controlled rate to a selected diameter.

17. The medical assembly of claim 14, wherein a first coefficient of friction exists between the flexible tension member and the channel, wherein a second coefficient of friction exists between the flexible tension member and the resistance feature, and wherein the first and the second coefficients of friction are different from each other.

18. The medical assembly of claim 12, wherein the flexible tension member comprises a first end portion coupled to the frame, and a second end portion opposite to the first end portion.

19. The medical assembly of claim 18, wherein, when depending on claim 13, the second end portion extends into the internal passage and is frictionally engaged with the friction surface of the resistance feature in the compressed state.

20. The medical assembly of claim 19, wherein, when the prosthetic heart valve self-expands from the compressed state to the expanded state, the second end portion is configured slide relative to the resistance feature such that a radially inwardly directed force applied to the frame by the flexible tension member is gradually lessened and the frame can expand at a controlled rate to a selected diameter.

21. The medical assembly of claim 18, wherein a first coefficient of friction exists between the flexible tension member and the frame, wherein a second coefficient of friction exists between the flexible tension member and the resistance feature, and wherein the first and the second coefficients of friction are different from each other.

22. The medical assembly of claim 17, wherein the second coefficient of friction is higher than the first coefficient of friction.
